SEQUENCE LISTING (48 sequences)

SEQ ID NO: 1 - DNA, 1377 bp, Homo sapiens
  misc_feature (1)..(1377): PU.1 Human - Full Sequence
aaaatcaggaacttgtgctggccctgcaatgtcaagggagggggctcacccagggctcctgtagctcagggggcaggcctgagccctgcacccgccccacgaccgtccagcccctgacggggcaccccatcctgaggggctctgcattggcccccaccgaggcaggggatctgaccgactcggagcccggctggatgttacaggcgtgcaaaatggaagggtttcccctcgtcccccctcagccatcagaagacctggtgccctatgacacggatctataccaacgccaaacgcacgagtattacccctatctcagcagtgatggggagagccatagcgaccattactgggacttccacccccaccacgtgcacagcgagttcgagagcttcgccgagaacaacttcacggagctccagagcgtgcagcccccgcagctgcagcagctctaccgccacatggagctggagcagatgcacgtcctcgatacccccatggtgccaccccatcccagtcttggccaccaggtctcctacctgccccggatgtgcctccagtacccatccctgtccccagcccagcccagctcagatgaggaggagggcgagcggcagagccccccactggaggtgtctgacggcgaggcggatggcctggagcccgggcctgggctcctgcctggggagacaggcagcaagaagaagatccgcctgtaccagttcctgttggacctgctccgcagcggcgacatgaaggacagcatctggtgggtggacaaggacaagggcaccttccagttctcgtccaagcacaaggaggcgctggcgcaccgctggggcatccagaagggcaaccgcaagaagatgacctaccagaagatggcgcgcgcgctgcgcaactacggcaagacgggcgaggtcaagaaggtgaagaagaagctcacctaccagttcagcggcgaagtgctgggccgcgggggcctggccgagcggcgccacccgccccactgagcccgcagcccccgccgggccccgccaggcctccccgctggccatagcattaagccctcgcccggcccggacacagggaggacgctcccggggcccagaggcaggactgtggcgggccgggcctcgcctcacccgccccctccccccactccaggccccctccacatcccgcttcgcctccctccaggactccaccccggctcccggacgccagctgggcgtcagaccccaccggggcaaccttgcagaggacgacccggggtactgccttgggagtctcaagtccgtatgtaaatcagatctcccctctcacccctcccacccattaacctcctcccaaaaaacaagtaaagttattctcaatcca

SEQ ID NO: 2 - DNA, 816 bp, Homo sapiens
  misc_feature (1)..(816): PU.1 Human - CDS - >NM_001080547.2195-1010 Homo sapiens Spi-1 proto-oncogene (SPI1), transcript variant 1, mRNA
atgttacaggcgtgcaaaatggaagggtttcccctcgtcccccctcagccatcagaagacctggtgccctatgacacggatctataccaacgccaaacgcacgagtattacccctatctcagcagtgatggggagagccatagcgaccattactgggacttccacccccaccacgtgcacagcgagttcgagagcttcgccgagaacaacttcacggagctccagagcgtgcagcccccgcagctgcagcagctctaccgccacatggagctggagcagatgcacgtcctcgatacccccatggtgccaccccatcccagtcttggccaccaggtctcctacctgccccggatgtgcctccagtacccatccctgtccccagcccagcccagctcagatgaggaggagggcgagcggcagagccccccactggaggtgtctgacggcgaggcggatggcctggagcccgggcctgggctcctgcctggggagacaggcagcaagaagaagatccgcctgtaccagttcctgttggacctgctccgcagcggcgacatgaaggacagcatctggtgggtggacaaggacaagggcaccttccagttctcgtccaagcacaaggaggcgctggcgcaccgctggggcatccagaagggcaaccgcaagaagatgacctaccagaagatggcgcgcgcgctgcgcaactacggcaagacgggcgaggtcaagaaggtgaagaagaagctcacctaccagttcagcggcgaagtgctgggccgcgggggcctggccgagcggcgccacccgccccactga

SEQ ID NO: 3 - protein, 271 aa, Homo sapiens
  MISC_FEATURE (1)..(271): PU.1 Human - Protein >NP_001074016.1 transcription factor PU.1 isoform 1 [Homo sapiens]
MLQACKMEGFPLVPPQPSEDLVPYDTDLYQRQTHEYYPYLSSDGESHSDHYWDFHPHHVHSEFESFAENNFTELQSVQPPQLQQLYRHMELEQMHVLDTPMVPPHPSLGHQVSYLPRMCLQYPSLSPAQPSSDEEEGERQSPPLEVSDGEADGLEPGPGLLPGETGSKKKIRLYQFLLDLLRSGDMKDSIWWVDKDKGTFQFSSKHKEALAHRWGIQKGNRKKMTYQKMARALRNYGKTGEVKKVKKKLTYQFSGEVLGRGGLAERRHPPH

SEQ ID NO: 4 - DNA, 1473 bp, Mus musculus
  misc_feature (1)..(1473): PU.1  Mouse - Full Sequence >NM_011355.2 Mus musculus spleen focus forming virus (SFFV) proviral integration oncogene (Spi1), transcript variant 2, mRNA
aagagatttatgcaaacgggctggggcggtgatgtcaccccaaggggactatctcccagtggcaggcccttcgataaaatcaggaacttgtgctggccctgcaatgtcaagggagggggctcacccagggctcctgtagctcagggggcaggcctgagccctgcgtctgacccacgaccgtccagtcccccgacggggcacctggtcctgagggggatccgccttgatccccaccgaagcaggggatctgaccaacctggagctcagctggatgttacaggcgtgcaaaatggaagggttttccctcaccgcccctccatcggatgacttggttacttacgattcagagctataccaacgtccaatgcatgactactactccttcgtgggcagcgatggagaaagccatagcgatcactactgggatttctccgcacaccatgtccacaacaacgagtttgagaacttccctgagaaccacttcacagagctgcagagtgtgcagcccccgcagctacagcagctctatcgccacatggagctggaacagatgcacgtcctcgatactcccatggtgccaccccacaccggcctcagtcaccaggtttcctacatgccccggatgtgcttcccttatcaaaccttgtccccagcccaccagcagagctcagatgaggaggagggtgagaggcagagccctcccctggaggtgtctgatggagaagctgatggcttggagcctgggccaggtcttctgcacggggagacaggcagcaagaaaaagattcgcctgtaccagttcctgctggacctgctgcgcagcggcgacatgaaggacagcatctggtgggtggacaaggacaaaggtaccttccagttctcgtccaagcacaaggaggcgctggcgcaccgctggggcatccagaagggcaaccgcaagaagatgacctaccagaagatggcgcgcgcgctgcgcaactacggcaagacaggcgaggtgaagaaagtcaagaagaagctcacctaccagttcagcggcgaggtgctgggccgtgggggcctggccgagcggcgcctcccgccccactgatcgcccgcagagaccgccaggctcctggaccccgccggccatagcattaacccgtcgcccggcccggacacagggaggacattcccagggccgaggcaggactgggggcccggcctcgccctcccatgcccggcctggcccgccccacccgctttgcctcccaccaggactctagcccgctccaagggccgcctgggcctcggacctcaaccgagggtcagcctggcttagtggccacggtgcttccttgggagtctggcgctggcacctttttgtatattgaatgctttttaaaaagctcttcctccccaccccctcattagtcactaaagacaagtaaaattattgacagctattctcccagaaaaaaaaaaaaaaaaaaa

SEQ ID NO: 5 - DNA, 819 bp, Mus musculus
  misc_feature (1)..(819): PU.1 Mouse CDS >NM_011355.2272-1090 Mus musculus spleen focus forming virus (SFFV) proviral integration oncogene (Spi1), transcript variant 2, mRNA
atgttacaggcgtgcaaaatggaagggttttccctcaccgcccctccatcggatgacttggttacttacgattcagagctataccaacgtccaatgcatgactactactccttcgtgggcagcgatggagaaagccatagcgatcactactgggatttctccgcacaccatgtccacaacaacgagtttgagaacttccctgagaaccacttcacagagctgcagagtgtgcagcccccgcagctacagcagctctatcgccacatggagctggaacagatgcacgtcctcgatactcccatggtgccaccccacaccggcctcagtcaccaggtttcctacatgccccggatgtgcttcccttatcaaaccttgtccccagcccaccagcagagctcagatgaggaggagggtgagaggcagagccctcccctggaggtgtctgatggagaagctgatggcttggagcctgggccaggtcttctgcacggggagacaggcagcaagaaaaagattcgcctgtaccagttcctgctggacctgctgcgcagcggcgacatgaaggacagcatctggtgggtggacaaggacaaaggtaccttccagttctcgtccaagcacaaggaggcgctggcgcaccgctggggcatccagaagggcaaccgcaagaagatgacctaccagaagatggcgcgcgcgctgcgcaactacggcaagacaggcgaggtgaagaaagtcaagaagaagctcacctaccagttcagcggcgaggtgctgggccgtgggggcctggccgagcggcgcctcccgccccactga

SEQ ID NO: 6 - protein, 272 aa, Mus musculus
  MISC_FEATURE (1)..(272): PU.1 Mouse - Protein >NP_035485.1 transcription factor PU.1 isoform 2 [Mus musculus]
MLQACKMEGFSLTAPPSDDLVTYDSELYQRPMHDYYSFVGSDGESHSDHYWDFSAHHVHNNEFENFPENHFTELQSVQPPQLQQLYRHMELEQMHVLDTPMVPPHTGLSHQVSYMPRMCFPYQTLSPAHQQSSDEEEGERQSPPLEVSDGEADGLEPGPGLLHGETGSKKKIRLYQFLLDLLRSGDMKDSIWWVDKDKGTFQFSSKHKEALAHRWGIQKGNRKKMTYQKMARALRNYGKTGEVKKVKKKLTYQFSGEVLGRGGLAERRLPPH

SEQ ID NO: 7 - DNA, 5314 bp, Homo sapiens
  misc_feature (1)..(5314): IRF4 Human - Full Sequence >NM_002460.4 Homo sapiens interferon regulatory factor 4 (IRF4), transcript variant 1, mRNA
agtttcaccgctcgatcttgggacccaccgctgccctcagctccgagtccagggcgagtgcagagcagagcgggcggaggaccccgggcgcgggcgcggacggcacgcggggcatgaacctggagggcggcggccgaggcggagagttcggcatgagcgcggtgagctgcggcaacgggaagctccgccagtggctgatcgaccagatcgacagcggcaagtaccccgggctggtgtgggagaacgaggagaagagcatcttccgcatcccctggaagcacgcgggcaagcaggactacaaccgcgaggaggacgccgcgctcttcaaggcttgggcactgtttaaaggaaagttccgagaaggcatcgacaagccggaccctcccacctggaagacgcgcctgcggtgcgctttgaacaagagcaatgactttgaggaactggttgagcggagccagctggacatctcagacccgtacaaagtgtacaggattgttcctgagggagccaaaaaaggagccaagcagctcaccctggaggacccgcagatgtccatgagccacccctacaccatgacaacgccttacccttcgctcccagcccagcaggttcacaactacatgatgccacccctcgaccgaagctggagggactacgtcccggatcagccacacccggaaatcccgtaccaatgtcccatgacgtttggaccccgcggccaccactggcaaggcccagcttgtgaaaatggttgccaggtgacaggaaccttttatgcttgtgccccacctgagtcccaggctcccggagtccccacagagccaagcataaggtctgccgaagccttggcgttctcagactgccggctgcacatctgcctgtactaccgggaaatcctcgtgaaggagctgaccacgtccagccccgagggctgccggatctcccatggacatacgtatgacgccagcaacctggaccaggtcctgttcccctacccagaggacaatggccagaggaaaaacattgagaagctgctgagccacctggagaggggcgtggtcctctggatggcccccgacgggctctatgcgaaaagactgtgccagagcaggatctactgggacgggcccctggcgctgtgcaacgaccggcccaacaaactggagagagaccagacctgcaagctctttgacacacagcagttcttgtcagagctgcaagcgtttgctcaccacggccgctccctgccaagattccaggtgactctatgctttggagaggagtttccagaccctcagaggcaaagaaagctcatcacagctcacgtagaacctctgctagccagacaactatattattttgctcaacaaaacagtggacatttcctgaggggctacgatttaccagaacacatcagcaatccagaagattaccacagatctatccgccattcctctattcaagaatgaaaaatgtcaagatgagtggttttctttttcctttttttttttttttttttgatacggggatacggggtcttgctctgtctcccaggctggagtgcagtgacacaatctcagctcactgtgacctccgcctcctgggttcaagagactctcctgcctcagcctccctggtagctgggattacaggtgtgagccactgcacccacccaagacaagtgattttcattgtaaatatttgactttagtgaaagcgtccaattgactgccctcttactgttttgaggaattcagaagtggagatttcagttcagcggttgaggagaattgcggcgagacaagcatggaaaatcagtgacatctgattggcagatgagcttatttcaaaaggaagggtggctttgcatttcttgtgttctgtagactgccatcattgatgatcactgtgaaaattgaccaagtgatgtgtttacatttactgaaatgcgctctttaatttgttgtagattaggtcttgctggaagacagagaaaacttgcctttcagtattgacactgactagagtgatgactgcttgtaggtatgtctgtgccatttctcagggaagtaagatgtaaattgaagaagcctcacacgtaaaagaaatgtattaatgtatgtaggagctgcagttcttgtggaagacacttgctgagtgaaggaaatgaatctttgactgaagccgtgcctgtagccttggggaggcccatcccccacctgccagcggtttcctggtgtgggtccctctgccccgccctccttcccattggctttctctccttggcctttcctggaagccagttagtaaacttcctattttcttgagtcaaaaaacatgagcgctactcttggatgggacatttttgtctgtcctacaatctagtaatgtctaagtaatggttaagttttcttgtttctgcatctttttgaccctcattctttagagatgctaaaattcttcgcataaagaagaagaaattaaggaacataaatcttaatacttgaactgttgcccttctgtccaagtacttaactatctgttcccttcctctgtgccacgctcctctgtttgtttggctgtccagcgatcagccatggcgacactaaaggaggaggagccggggactcccaggctggagagcactgccaggacccaccactggaagcaggatggagctgactacggaactgcacactcagtgggctgtttctgcttatttcatctgttctatgcttcctcgtgccaattatagtttgacagggccttaaaattacttggctttttccaaatgcttctatttatagaatcccaaagacctccacttgcttaagtatacctatcacttacatttttgtggttttgagaaagtacagcagtagactggggcgtcacctccaggccgtttctcatactacaggatatttactattactcccaggatcagcagaagattgcgtagctctcaaatgtgtgttcctgcttttctaatggatattttaaattcattcaacaagcacctagtaagtgcctgctgtatccctacattacacagttcagcctttatcaagcttagtgagcagtgagcactgaaacattattttttaatgtttaaaaagtttctaatattaaagtcagaatattaatacaattaatattaatattaactacagaaaagacaaacagtagagaacagcaaaaaaataaaaaggatctccttttttcccagcccaaattctcctctctaaaagtgtccacaagaaggggtgtttattcttccaacacatttcacttttctgtaaatatacataaacttaaaaagaaaacctcatggagtcatcttgcacacactttcatgcagtgctctttgtagctaacagtgaagatttacctcgttctgctcagaggccttgctgtggagctccactgccatgtacccagtagggtttgacatttcattagccatgcaacatggatatgtattgggcagcagactgtgtttcgtgaactgcagtgatgtatacatcttatagatgcaaagtattttggggtatattatcctaagggaagataaagatgatattaagaactgctgtttcacggggcccttacctgtgaccctctttgctgaagaatatttaaccccacacagcacttcaaagaagctgtcttggaagtctgtctcaggagcaccctgtcttcttaattctccaagcggatgctccatttcaattgctttgtgacttcttcttctttgtttttttaaatattatgctgctttaacagtggagctgaattttctggaaaatgcttcttggctggggccactacctcctttcctatctttacatctatgtgtatgttgactttttaaaattctgagtgatccagggtatgacctagggaatgaactagctatgaaatactcagggttaggaatcctagcacttgtctcaggactctgaaaaggaacggcttcctcattccttgtcttgataaagtggaattggcaaactagaatttagtttgtactcagtggacagtgctgttgaagatttgaggacttgttaaagagcactgggtcatatggaaaaaatgtatgtgtctcccaggtgcatttcttggtttatgtcttgttcttgagattttgtatatttaggaaaacctcaagcagtaattaatatctcctggaacactatagagaaccaagtgaccgactcatttacaactgaaacctaggaagcccctgagtcctgagcgaaaacaggagagttagtcgccctacagaaaacccagctagactattgggtatgaactaaaaagagactgtgccatggtgagaaaaatgtaaaatcctacagtgaaatgagcagcccttacagtattgttaccaccaagggcaggtaggtattagtgtttgaaaaagctggtctttgagcgagggcataaatacagctagccccaggggtggaacaactctgggagtcttgggtactcgcacctcttggctttgttgatgctccgccaggaaggccacttgtgtgtgcgtgtcagttacttttttagtaacaattcagatccagtgtaaacttccgttcattgctctccagtcacatgcccccacttccccacaggtgaaagtttttctgaaagtgttgggattggttaaggtctttatttgtattacgtatctccccaagtcctctgtggccagctgcatctgtctgaatggtgcgtgaaggctctcagaccttacacaccattttgtaagttatgttttacatgccccgtttttgagactgatctcgatgcaggtggatctccttgagatcctgatagcctgttacaggaatgaagtaaaggtcagtttttttttgtattgattttcacagctttgaggaacatgcataagaaatgtagctgaagtagaggggacgtgagagaagggccaggccggcaggccaaccctcctccaatggaaattcccgtgttgcttcaaactgagacagatgggacttaacaggcaatggggtccacttccccctcttcagcatcccccgtaccccactttctgctgaaagaactgccagcaggtaggaccccagaggcccccaaatgaaagcttgaatttcccctactggctctgcgttttgctgagatctgtaggaaaggatgcttcacaaactgaggtagataatgctatgctgtcgttggtatacatcatgaatttttatgtaaattgctctgcaaagcaaattgatatgtttgataaatttatgtttttaggtaaataaaaacttttaaaaatttgtta

SEQ ID NO: 8 - DNA, 1356 bp, Homo sapiens
  misc_feature (1)..(1356): IRF4 Human CDS >NM_002460.4114-1469 Homo sapiens interferon regulatory factor 4 (IRF4), transcript variant 1, mRNA
atgaacctggagggcggcggccgaggcggagagttcggcatgagcgcggtgagctgcggcaacgggaagctccgccagtggctgatcgaccagatcgacagcggcaagtaccccgggctggtgtgggagaacgaggagaagagcatcttccgcatcccctggaagcacgcgggcaagcaggactacaaccgcgaggaggacgccgcgctcttcaaggcttgggcactgtttaaaggaaagttccgagaaggcatcgacaagccggaccctcccacctggaagacgcgcctgcggtgcgctttgaacaagagcaatgactttgaggaactggttgagcggagccagctggacatctcagacccgtacaaagtgtacaggattgttcctgagggagccaaaaaaggagccaagcagctcaccctggaggacccgcagatgtccatgagccacccctacaccatgacaacgccttacccttcgctcccagcccagcaggttcacaactacatgatgccacccctcgaccgaagctggagggactacgtcccggatcagccacacccggaaatcccgtaccaatgtcccatgacgtttggaccccgcggccaccactggcaaggcccagcttgtgaaaatggttgccaggtgacaggaaccttttatgcttgtgccccacctgagtcccaggctcccggagtccccacagagccaagcataaggtctgccgaagccttggcgttctcagactgccggctgcacatctgcctgtactaccgggaaatcctcgtgaaggagctgaccacgtccagccccgagggctgccggatctcccatggacatacgtatgacgccagcaacctggaccaggtcctgttcccctacccagaggacaatggccagaggaaaaacattgagaagctgctgagccacctggagaggggcgtggtcctctggatggcccccgacgggctctatgcgaaaagactgtgccagagcaggatctactgggacgggcccctggcgctgtgcaacgaccggcccaacaaactggagagagaccagacctgcaagctctttgacacacagcagttcttgtcagagctgcaagcgtttgctcaccacggccgctccctgccaagattccaggtgactctatgctttggagaggagtttccagaccctcagaggcaaagaaagctcatcacagctcacgtagaacctctgctagccagacaactatattattttgctcaacaaaacagtggacatttcctgaggggctacgatttaccagaacacatcagcaatccagaagattaccacagatctatccgccattcctctattcaagaatga

SEQ ID NO: 9 - protein, 451 aa, Homo sapiens
  MISC_FEATURE (1)..(451): IRF4 Human Protein >NP_002451.2 interferon regulatory factor 4 isoform 1 [Homo sapiens]
MNLEGGGRGGEFGMSAVSCGNGKLRQWLIDQIDSGKYPGLVWENEEKSIFRIPWKHAGKQDYNREEDAALFKAWALFKGKFREGIDKPDPPTWKTRLRCALNKSNDFEELVERSQLDISDPYKVYRIVPEGAKKGAKQLTLEDPQMSMSHPYTMTTPYPSLPAQQVHNYMMPPLDRSWRDYVPDQPHPEIPYQCPMTFGPRGHHWQGPACENGCQVTGTFYACAPPESQAPGVPTEPSIRSAEALAFSDCRLHICLYYREILVKELTTSSPEGCRISHGHTYDASNLDQVLFPYPEDNGQRKNIEKLLSHLERGVVLWMAPDGLYAKRLCQSRIYWDGPLALCNDRPNKLERDQTCKLFDTQQFLSELQAFAHHGRSLPRFQVTLCFGEEFPDPQRQRKLITAHVEPLLARQLYYFAQQNSGHFLRGYDLPEHISNPEDYHRSIRHSSIQE

SEQ ID NO: 10 - DNA, 4760 bp, Mus musculus
  misc_feature (1)..(4760): IRF4 Mouse - Full Sequence >NM_013674.2 Mus musculus interferon regulatory factor 4 (Irf4), transcript variant 1, mRNA
tttcctagtttcaccacttgaacttgggaccctttgctgccctcagctaagagtgcgggtgagcgcacaagcccaggaggaggtccgcacgcgtcatgaacttggagacgggcagccggggctcagagttcggcatgagcgcagtgagctgcggcaatgggaaactccgacagtggttgatcgaccagatcgacagcggcaagtaccccgggctggtgtgggagaacgaggagaagagcgtcttccgcatcccgtggaaacacgcgggcaagcaggactacaatcgtgaggaggacgctgccctcttcaaggcttgggcattgtttaaaggcaagttccgagaagggatcgacaagccagatcctcctacttggaagacaagattacgatgtgctctgaacaagagcaatgactttgaggaattggtcgagaggagccagctggatatctctgacccatacaaggtgtacaggattgttccagagggagccaaaaaaggagcaaagcagctcactttggatgacacacagatggccatgggccacccctaccccatgacagcaccttatggctctctgccagcccagcaggttcataactacatgatgccaccccatgacaggagctggagggattatgcccctgaccagtcacacccagaaatcccatatcaatgtcctgtgacgtttggcccacgaggccaccactggcaaggcccatcttgtgaaaatggttgccaggtgacaggaaccttttatgcttgtgccccacctgagtcccaggctcctggaatccccattgagccaagcataaggtctgctgaagccttggcgctctcagactgccggctgcatatctgcctgtattaccgggacatcctcgtgaaagagctgaccacgacgagccctgaaggctgccggatctcccacggacacacctatgatgttagcaacctggaccaggtcctgtttccctacccggacgacaatggacagaggaagaacattgagaagttgctgagccacctggagaggggactggtcctctggatggctccagatgggctttatgccaaaagactctgccagagtaggatctactgggatgggcccctggcactgtgcagcgatcggcccaacaagctagaaagagaccagacttgcaagctctttgacacacagcagtttctatcagagctgcaagtgtttgctcaccatggccggccagcaccgagattccaggtgactctgtgctttggtgaggagtttccagaccctcagagacagaggaagctcatcacagctcatgtggaacctctgctagccagacaactgtattactttgctcaacaaaacactggacatttcctgaggggctacgagttacctgaacacgttaccactccagattaccaccgctccctccgtcattcttccatccaagagtgagaagaaatactctgacagggcagccggctgctgccctttctctttggaagagctaagaagtgagtgggtttccacttgaagacaacaacagggctttgtgaggaaaaacagctgtatctgctcaacagaggagcttcccccagaagagtgcctgtcatccaggtcttgacaagtgccaggacttgggtgactgtgccctggcttataactgtgaaacttgatcagtgttgtgtttacatgtacttgaatgctggctttagcctggtatagatggacttttgcttgaagactgaaaacctgtgccagcatgaatccctgacaagagaagacatacgtattattggtccatttctcagggaagtaaagtctagactagagactacgctgtccactcacagagaaacgcattcctggatagaggagctgtaggttgcccagatcatgtccactgagtgaagggacttggttcttaggtccagtctaggctatcatgtcctgatcctgcactctctctgagtagaccctacatcctgtttgctccctcttcttgcccttccctaaaagccagctggtggactcttgttgccacatacttgagccaaaaaaccatcagggacatataagatgagacatgtttgcatctcctctgatctaacaacggctacaaagttttttgttgttgttgtctcccctttctgttttgcctctcattctttagaattgttgcttttttgtaaagaaaggaagagagagagaccgaatatactagagatagatttttaaaattctgtgccatttccccttctatttgttttgttggccatcagtggtccccatggcaacactggaagggcggagcctttgagcaggttgggaactgtcccggacaccagtggttcagtgtacagctgtagactcagtgagctgtttctgcttattttgtatattagatgcttccttgtgccaataatagtttgacagagtctcaaagctaccgggcttcttttccaacaacacattctttcacagaatcccaaagagtctcacttgccttcacaaccgtctgatcctttattgttactgttttgtttaggtggctttgccatgctctgggatttttctcctaccacaacatcattcctgactctttcgggctcagaaagagttaagcgctcttgcacacttggcacttacttctttttaattttttttttaacatgccactcttaatatttcttcttaagtctttcagcagcatacattcaataagcaatggaccattgggtgtctcatttcattgttttatttttaaaaaggaacctttgacaaataatacacattacagaaaatggaagcatttttcatagagaaggtaagaactgaaatgcattttatgttcgaatcctccttgctagtattagccccaagagttcagtctgtcctttaagcatgttttagttttcaatacatatatatgaagttgccctttcttagagagagagagagagatatgtgatgacactcttatgtacccaactatacagatttacctcatcttttgagtggcatgtggacttccactgctatgtgcctaggtgcagtttagacatcttcttagctacccatcccagcagcccactgagtctcgtgaactaaagtcacttatacatcttatagatgcaaaacattctggggtatattatcccaagggaagatgaaggcaaaatcgggaagcactcttcctgaggccccaagccacagctgtcctcctggagtatcttaaccatatctggcacttcaccaaggtcccttgcctggtcctgtatgaccatgactcttcagtccataagcagatgctttgtctcaattgccatgtgaagtcttttggtttttggtttttttccttaatgttaagctgttttaacagtgaagctgaattttctggaaaatgctgtatggttcgagcccccagttcatgtttcactttacagccatgcatgctgatgccttccatcttgacaaatggaatgatgggtaaacactcctgacacaaagaggaacaccttcctcgtcccttgctgaaacaaagtgcaatgcacacagtagaatttagtttgcttgcactggaagatgccgttgaagaggtaggctgaggaacactgtatcctagggagaaaatatgcaagtctcacgggtgctttctgttggcttgtttctggtccttgagcaggcttaagtatctagtttcaaactagaagccccaaagccctcagtcgttgtcctggacagagttggtagaccccacctgtagaaatccagatgcactctgggattggagcacagagaggcttgctctatcgtgagaatgcccagcctgcagaggcccaggtgcggtgcctgagtggctgtatgccagacacaggaacgccagcaggctgagtgtggtgactgatatgtgaggtgcccttgccctgcagacaggctgaatgtgagccccagcagcagaggattctgagagtcagggtggtgtcatgtcctacacaggaagagccttcaatattactttttaaacgaactttcccattgctgatattttgataaaaacccacacaaggtttaattacatgccccctcatccctctccacacagtcttgcccatcccactggtgaattgttgttgttcttgttgttgttttttgttattggtgaaaattgtgaaattgttaaggtctctgtttttgttatttatctccctaaatcacatggctgctgcatacattgcaatgttgccctaccacatgtatgtttttgcaagttgcttcttcactcattgaagaagcctgcaccgtaagtgaaactcacttagtccttgctaattgctgctcactgtcacgtgaacgcagagaaggctgattttcctggacccattttcagtgctttgaggaacatgagtccagagtatattgaagtagaagacagtgtttggcaagccagttcttattagacactgctacaggcttcacaatcttcaaggtggacccacctcatgtggaagggcatcattgcccctctgctcccatgcgctgtgcacccccttcacagtgaaagacctgctggggaccaggactcagagatctctggcttcgagctcatatcttggcttcctgtcatgtctaagcccgacaggaaagtgtggcaccatcctgcctgcggcgttggatggataggccatggattttgctgtaaacttgcttcacaaggaaagctgtgtgttttaaaatatttgataaataaaggatttctagaaagat

SEQ ID NO: 11 - DNA, 1353 bp, Mus musculus
  misc_feature (1)..(1353): IRF4  Mouse CDS >NM_013674.296-1448 Mus musculus interferon regulatory factor 4 (Irf4), transcript variant 1, mRNA
atgaacttggagacgggcagccggggctcagagttcggcatgagcgcagtgagctgcggcaatgggaaactccgacagtggttgatcgaccagatcgacagcggcaagtaccccgggctggtgtgggagaacgaggagaagagcgtcttccgcatcccgtggaaacacgcgggcaagcaggactacaatcgtgaggaggacgctgccctcttcaaggcttgggcattgtttaaaggcaagttccgagaagggatcgacaagccagatcctcctacttggaagacaagattacgatgtgctctgaacaagagcaatgactttgaggaattggtcgagaggagccagctggatatctctgacccatacaaggtgtacaggattgttccagagggagccaaaaaaggagcaaagcagctcactttggatgacacacagatggccatgggccacccctaccccatgacagcaccttatggctctctgccagcccagcaggttcataactacatgatgccaccccatgacaggagctggagggattatgcccctgaccagtcacacccagaaatcccatatcaatgtcctgtgacgtttggcccacgaggccaccactggcaaggcccatcttgtgaaaatggttgccaggtgacaggaaccttttatgcttgtgccccacctgagtcccaggctcctggaatccccattgagccaagcataaggtctgctgaagccttggcgctctcagactgccggctgcatatctgcctgtattaccgggacatcctcgtgaaagagctgaccacgacgagccctgaaggctgccggatctcccacggacacacctatgatgttagcaacctggaccaggtcctgtttccctacccggacgacaatggacagaggaagaacattgagaagttgctgagccacctggagaggggactggtcctctggatggctccagatgggctttatgccaaaagactctgccagagtaggatctactgggatgggcccctggcactgtgcagcgatcggcccaacaagctagaaagagaccagacttgcaagctctttgacacacagcagtttctatcagagctgcaagtgtttgctcaccatggccggccagcaccgagattccaggtgactctgtgctttggtgaggagtttccagaccctcagagacagaggaagctcatcacagctcatgtggaacctctgctagccagacaactgtattactttgctcaacaaaacactggacatttcctgaggggctacgagttacctgaacacgttaccactccagattaccaccgctccctccgtcattcttccatccaagagtga

SEQ ID NO: 12 - protein, 450 aa, Mus musculus
  MISC_FEATURE (1)..(450): IRF4 Mouse Protein >NP_038702.1 interferon regulatory factor 4 isoform a [Mus musculus]
MNLETGSRGSEFGMSAVSCGNGKLRQWLIDQIDSGKYPGLVWENEEKSVFRIPWKHAGKQDYNREEDAALFKAWALFKGKFREGIDKPDPPTWKTRLRCALNKSNDFEELVERSQLDISDPYKVYRIVPEGAKKGAKQLTLDDTQMAMGHPYPMTAPYGSLPAQQVHNYMMPPHDRSWRDYAPDQSHPEIPYQCPVTFGPRGHHWQGPSCENGCQVTGTFYACAPPESQAPGIPIEPSIRSAEALALSDCRLHICLYYRDILVKELTTTSPEGCRISHGHTYDVSNLDQVLFPYPDDNGQRKNIEKLLSHLERGLVLWMAPDGLYAKRLCQSRIYWDGPLALCSDRPNKLERDQTCKLFDTQQFLSELQVFAHHGRPAPRFQVTLCFGEEFPDPQRQRKLITAHVEPLLARQLYYFAQQNTGHFLRGYELPEHVTTPDYHRSLRHSSIQE

SEQ ID NO: 13 - DNA, 5148 bp, Homo sapiens
  misc_feature (1)..(5148): PRDM1 Human - Full Sequence >NM_001198.4 Homo sapiens PR/SET domain 1 (PRDM1), transcript variant 1, mRNA
aacacagacaaagtgctgccgtgacactcggccctccagtgttgcggagaggcaagagcagcgaccgcggcacctgtccgcccggagctgggacgcgggcgcccgggcggccggacgaagcgaggagggaccgccgaggtgcgcgtctgtgcggctcagcctggcgggggacgcggggagaatgtggactgggtagagatgaacgagacttttctcagatgttggatatttgcttggaaaaacgtgtgggtacgaccttggctgcccccaagtgtaactccagcactgtgaggtttcagggattggcagaggggaccaaggggaccatgaaaatggacatggaggatgcggatatgactctgtggacagaggctgagtttgaagagaagtgtacatacattgtgaacgaccacccctgggattctggtgctgatggcggtacttcggttcaggcggaggcatccttaccaaggaatctgcttttcaagtatgccaccaacagtgaagaggttattggagtgatgagtaaagaatacataccaaagggcacacgttttggacccctaataggtgaaatctacaccaatgacacagttcctaagaacgccaacaggaaatatttttggaggatctattccagaggggagcttcaccacttcattgacggctttaatgaagagaaaagcaactggatgcgctatgtgaatccagcacactctccccgggagcaaaacctggctgcgtgtcagaacgggatgaacatctacttctacaccattaagcccatccctgccaaccaggaacttcttgtgtggtattgtcgggactttgcagaaaggcttcactacccttatcccggagagctgacaatgatgaatctcacacaaacacagagcagtctaaagcaaccgagcactgagaaaaatgaactctgcccaaagaatgtcccaaagagagagtacagcgtgaaagaaatcctaaaattggactccaacccctccaaaggaaaggacctctaccgttctaacatttcacccctcacatcagaaaaggacctcgatgactttagaagacgtgggagccccgaaatgcccttctaccctcgggtcgtttaccccatccgggcccctctgccagaagactttttgaaagcttccctggcctacgggatcgagagacccacgtacatcactcgctcccccattccatcctccaccactccaagcccctctgcaagaagcagccccgaccaaagcctcaagagctccagccctcacagcagccctgggaatacggtgtcccctgtgggccccggctctcaagagcaccgggactcctacgcttacttgaacgcgtcctacggcacggaaggtttgggctcctaccctggctacgcacccctgccccacctcccgccagctttcatcccctcgtacaacgctcactaccccaagttcctcttgcccccctacggcatgaattgtaatggcctgagcgctgtgagcagcatgaatggcatcaacaactttggcctcttcccgaggctgtgccctgtctacagcaatctcctcggtgggggcagcctgccccaccccatgctcaaccccacttctctcccgagctcgctgccctcagatggagcccggaggttgctccagccggagcatcccagggaggtgcttgtcccggcgccccacagtgccttctcctttaccggggccgccgccagcatgaaggacaaggcctgtagccccacaagcgggtctcccacggcgggaacagccgccacggcagaacatgtggtgcagcccaaagctacctcagcagcgatggcagcccccagcagcgacgaagccatgaatctcattaaaaacaaaagaaacatgaccggctacaagacccttccctacccgctgaagaagcagaacggcaagatcaagtacgaatgcaacgtttgcgccaagactttcggccagctctccaatctgaaggtccacctgagagtgcacagtggagaacggcctttcaaatgtcagacttgcaacaagggctttactcagctcgcccacctgcagaaacactacctggtacacacgggagaaaagccacatgaatgccaggtctgccacaagagatttagcagcaccagcaatctcaagacccacctgcgactccattctggagagaaaccataccaatgcaaggtgtgccctgccaagttcacccagtttgtgcacctgaaactgcacaagcgtctgcacacccgggagcggccccacaagtgctcccagtgccacaagaactacatccatctctgtagcctcaaggttcacctgaaagggaactgcgctgcggccccggcgcctgggctgcccttggaagatctgacccgaatcaatgaagaaatcgagaagtttgacatcagtgacaatgctgaccggctcgaggacgtggaggatgacatcagtgtgatctctgtagtggagaaggaaattctggccgtggtcagaaaagagaaagaagaaactggcctgaaagtgtctttgcaaagaaacatggggaatggactcctctcctcagggtgcagcctttatgagtcatcagatctacccctcatgaagttgcctcccagcaacccactacctctggtacctgtaaaggtcaaacaagaaacagttgaaccaatggatccttaagattttcagaaaacacttattttgtttcttaagttatgacttggtgagtcagggtgcctgtaggaagtggcttgtacataatcccagctctgcaaagctctctcgacagcaaatggtttcccctcacctctggaattaaagaaggaactccaaagttactgaaatctcagggcatgaacaaggcaaaggccatatatatatatatatatatatctgtatacatattatatatacttatttacacctgtgtctatatatttgcccctgtgtattttgaatatttgtgtggacatgtttgcatagccttcccattactaagactattacctagtcataattattttttcaatgataatccttcataatttattatacaatttatcattcagaaagcaataattaaaaaagtttacaatgactggaaagattccttgtaatttgagtataaatgtatttttgtcttgtggccattctttgtagataatttctgcacatctgtataagtacctaagatttagttaaacaaatatatgacttcagtcaacctctctctctaataatggtttgaaaatgaggtttgggtaattgccaatgttggacagttgatgtgttcattcctgggatcctatcatttgaacagcattgtacataacttgggggtatgtgtgcaggattacccaagaataacttaagtagaagaaacaagaaagggaatcttgtatatttttgttgatagttcatgtttttcccccagccacaattttaccggaagggtgacaggaaggctttaccaacctgtctctccctccaaaagagcagaatcctcccaccgccctgccctccccaccgagtcctgtggccattcagagcggccacatgacttttgcatccattgtattatcagaaaatgtgaagaagaaaaaaatgccatgttttaaaaccactgcgaaaatttccccaaagcataggtggctttgtgtgtgtgcgatttgggggcttgagtctgggtggtgttttgttgttggtttttgttgctttttttttttttttttttttaatgtcaaaattgcacaaacatggtgctctaccaggaaggattcgaggtagataggctcaggccacactttaaaaacaaacacacaaacaacaaaaaacgggtattctagtcatcttggggtaaaagcgggtaatgaacattcctatccccaacacatcaattgtattttttctgtaaaactcagattttcctcagtatttgtgtttttacattttatggttaatttaatggaagatgaaagggcattgcaaagttgttcaacaacagttacctcattgagtgtgtccagtagtgcaggaaatgatgtcttatctaatgatttgcttctctagaggagaaaccgagtaaatgtgctccagcaagatagactttgtgttattctatcttttattctgctaagcccaaagattacatgttggtgttcaaagtgtagcaaaaaatgatgtatatttataaatctatttataccactatatcatatgtatatatatttataaccacttaaattgtgagccaagccatgtaaaagatctactttttctaagggcaaaaaaaaaaaaaaaaaaaaaagaacactcctttctgagactttgcttaatacttggtgacctcacaatcacgtcggtatgattgggcacccttgcctactgtaagagaccctaaaaccttggtgcagtggtggggaccacaaaacaaccagggaggaagagatacatcattttttagtattaaggaccatctaagacagctctattttttttttgccactttatgattatgtggtcacacccaagtcacagaaataaaaaactgactttaccgctgcaatttttctgttttcctccttactaaatactgatacattactccaatctattttataattatatttgacattttgttcacatcaactaatgttcacctgtagaagagaacaaatttcgaataatccagggaaacccaagagccttactggtcttctgtaacttccaagactgacagctttttatgtatcagtgtttgataaacacagtccttaactgaaggtaaaccaaagcatcacgttgacattagaccaaatacttttgattcccaactactcgtttgttctttttctccttttgtgctttcccatagtgagaatttttataaagacttcttgcttctctcaccatccatccttctcttttctgcctcttacatgtgaatgttgagcccacaatcaacagtggttttattttttcctctactcaaagttaaaactgaccaaagttactggctttttactttgctagaacaacaaactatcttatgtttacatactggtttacaatgttatttatgtgcaaattgtcaaaatgtaaattaaatataaatgttcatgctttaccaaaa

SEQ ID NO: 14 - DNA, 2478 bp, Homo sapiens
  misc_feature (1)..(2478): PRDM1 Human CDS >NM_001198.4219-2696 Homo sapiens PR/SET domain 1 (PRDM1), transcript variant 1, mRNA
atgttggatatttgcttggaaaaacgtgtgggtacgaccttggctgcccccaagtgtaactccagcactgtgaggtttcagggattggcagaggggaccaaggggaccatgaaaatggacatggaggatgcggatatgactctgtggacagaggctgagtttgaagagaagtgtacatacattgtgaacgaccacccctgggattctggtgctgatggcggtacttcggttcaggcggaggcatccttaccaaggaatctgcttttcaagtatgccaccaacagtgaagaggttattggagtgatgagtaaagaatacataccaaagggcacacgttttggacccctaataggtgaaatctacaccaatgacacagttcctaagaacgccaacaggaaatatttttggaggatctattccagaggggagcttcaccacttcattgacggctttaatgaagagaaaagcaactggatgcgctatgtgaatccagcacactctccccgggagcaaaacctggctgcgtgtcagaacgggatgaacatctacttctacaccattaagcccatccctgccaaccaggaacttcttgtgtggtattgtcgggactttgcagaaaggcttcactacccttatcccggagagctgacaatgatgaatctcacacaaacacagagcagtctaaagcaaccgagcactgagaaaaatgaactctgcccaaagaatgtcccaaagagagagtacagcgtgaaagaaatcctaaaattggactccaacccctccaaaggaaaggacctctaccgttctaacatttcacccctcacatcagaaaaggacctcgatgactttagaagacgtgggagccccgaaatgcccttctaccctcgggtcgtttaccccatccgggcccctctgccagaagactttttgaaagcttccctggcctacgggatcgagagacccacgtacatcactcgctcccccattccatcctccaccactccaagcccctctgcaagaagcagccccgaccaaagcctcaagagctccagccctcacagcagccctgggaatacggtgtcccctgtgggccccggctctcaagagcaccgggactcctacgcttacttgaacgcgtcctacggcacggaaggtttgggctcctaccctggctacgcacccctgccccacctcccgccagctttcatcccctcgtacaacgctcactaccccaagttcctcttgcccccctacggcatgaattgtaatggcctgagcgctgtgagcagcatgaatggcatcaacaactttggcctcttcccgaggctgtgccctgtctacagcaatctcctcggtgggggcagcctgccccaccccatgctcaaccccacttctctcccgagctcgctgccctcagatggagcccggaggttgctccagccggagcatcccagggaggtgcttgtcccggcgccccacagtgccttctcctttaccggggccgccgccagcatgaaggacaaggcctgtagccccacaagcgggtctcccacggcgggaacagccgccacggcagaacatgtggtgcagcccaaagctacctcagcagcgatggcagcccccagcagcgacgaagccatgaatctcattaaaaacaaaagaaacatgaccggctacaagacccttccctacccgctgaagaagcagaacggcaagatcaagtacgaatgcaacgtttgcgccaagactttcggccagctctccaatctgaaggtccacctgagagtgcacagtggagaacggcctttcaaatgtcagacttgcaacaagggctttactcagctcgcccacctgcagaaacactacctggtacacacgggagaaaagccacatgaatgccaggtctgccacaagagatttagcagcaccagcaatctcaagacccacctgcgactccattctggagagaaaccataccaatgcaaggtgtgccctgccaagttcacccagtttgtgcacctgaaactgcacaagcgtctgcacacccgggagcggccccacaagtgctcccagtgccacaagaactacatccatctctgtagcctcaaggttcacctgaaagggaactgcgctgcggccccggcgcctgggctgcccttggaagatctgacccgaatcaatgaagaaatcgagaagtttgacatcagtgacaatgctgaccggctcgaggacgtggaggatgacatcagtgtgatctctgtagtggagaaggaaattctggccgtggtcagaaaagagaaagaagaaactggcctgaaagtgtctttgcaaagaaacatggggaatggactcctctcctcagggtgcagcctttatgagtcatcagatctacccctcatgaagttgcctcccagcaacccactacctctggtacctgtaaaggtcaaacaagaaacagttgaaccaatggatccttaa

SEQ ID NO: 15 - protein, 825 aa, Homo sapiens
  MISC_FEATURE (1)..(825): PRDM1 Human Protein >NP_001189.2 PR domain zinc finger protein 1 isoform 1 [Homo sapiens]
MLDICLEKRVGTTLAAPKCNSSTVRFQGLAEGTKGTMKMDMEDADMTLWTEAEFEEKCTYIVNDHPWDSGADGGTSVQAEASLPRNLLFKYATNSEEVIGVMSKEYIPKGTRFGPLIGEIYTNDTVPKNANRKYFWRIYSRGELHHFIDGFNEEKSNWMRYVNPAHSPREQNLAACQNGMNIYFYTIKPIPANQELLVWYCRDFAERLHYPYPGELTMMNLTQTQSSLKQPSTEKNELCPKNVPKREYSVKEILKLDSNPSKGKDLYRSNISPLTSEKDLDDFRRRGSPEMPFYPRVVYPIRAPLPEDFLKASLAYGIERPTYITRSPIPSSTTPSPSARSSPDQSLKSSSPHSSPGNTVSPVGPGSQEHRDSYAYLNASYGTEGLGSYPGYAPLPHLPPAFIPSYNAHYPKFLLPPYGMNCNGLSAVSSMNGINNFGLFPRLCPVYSNLLGGGSLPHPMLNPTSLPSSLPSDGARRLLQPEHPREVLVPAPHSAFSFTGAAASMKDKACSPTSGSPTAGTAATAEHVVQPKATSAAMAAPSSDEAMNLIKNKRNMTGYKTLPYPLKKQNGKIKYECNVCAKTFGQLSNLKVHLRVHSGERPFKCQTCNKGFTQLAHLQKHYLVHTGEKPHECQVCHKRFSSTSNLKTHLRLHSGEKPYQCKVCPAKFTQFVHLKLHKRLHTRERPHKCSQCHKNYIHLCSLKVHLKGNCAAAPAPGLPLEDLTRINEEIEKFDISDNADRLEDVEDDISVISVVEKEILAVVRKEKEETGLKVSLQRNMGNGLLSSGCSLYESSDLPLMKLPPSNPLPLVPVKVKQETVEPMDP

SEQ ID NO: 16 - DNA, 5221 bp, Mus musculus
  misc_feature (1)..(5221): PRDM1 Mouse - Full Sequence >NM_007548.4 Mus musculus PR domain containing 1, with ZNF domain (Prdm1), mRNA
gggggaagagtagtcagtcgctcgctcactcgctcgctcgcacagacactgctgcagtgacactcggccctccagtgtcgcggagacgcaagagcagcgcgcagcacctgtccgcccggagcgagcccggcccgcggccgtagaaaaggagggaccgccgaggtgcgcgtcagtactgctcagcccggcagggacgcgggaggatgtggactgggtggacatgagagaggcttatctcagatgttggatcttctcttggaaaaacgtgtgggtacgaccttggctgcccccaagtctagctccggctccgtgaagtttcaaggactggcagagactgggatcatgaaaatggacatggaggacgctgatatgactttgtggacagaggccgagtttgaagagaagtgtacatacatagtgaacgaccacccctgggattccggcgctgacgggggtacttctgttcaagccgaggcatccttaccaaggaacctgcttttcaagtatgctgccaacaacagcaaagaggttattggcgtggtaagtaaggagtacataccgaagggaacacgctttggacccctcatcggtgaagtctacactaatgacacagttcccaagaatgccaacaggaagtatttttggcggatctattccagagaggagttccaccacttcattgatggctttaatgaggagaaaagcaactggatgcgctacgtgaatccagctcactctgcccgggagcaaaacctggctgcctgtcagaacgggatgaacatctacttctacactattaagcctatccctgccaaccaggaacttcttgtgtggtattgtcgggactttgcggagaggctccactacccttatcctggagagctcacagtgataaatctcacacaaacggaaagcaacccaaagcaatacagtagtgagaaaaatgaactctacccaaagagtgtccccaagagagagtacagcgtgaaagaaattctaaaactggactccaatccctccaaaaggaaggacatctaccgttccaacatttcacccttcactttagaaaaggacatggatggctttcggaaaaatgggagccccgacatgcccttctaccctcgggtggtttatcctatccgggcacctctgccagaagactttttgaaagcgtccctggcctatgggatggagagacccacctacataactcacagtccccttccgtcttccacaactccaagtccccctgcgagcagcagcccggagcagagccttaagagctccagcccccacagcagcccgggaaacacggtgtcacccctggcgccaggcctcccagaacaccgggactcctactcctacttgaatgtttcctatggttccgagggcctgggctcctaccctggctatgcacctgccccccacctcccaccagctttcattccttcttacaatgctcactaccccaagttcctgttgccaccgtacggcattagttccaatggcttgagcaccatgaacaacatcaatggtatcaacaacttcagcctcttccctaggttgtatcccgtctacagtaacctccttagtggcagcagcctgcctcatcccatgctcaatccagcttccctaccgagttccctgcctaccgatggagcccggaggctgcttccaccggagcaccccaaagaggtgcttatcccagcaccccacagtgccttctcccttaccggggctgcagccagcatgaaggacgagagtagtccccccagcggatctccaacggcgggaactgcagccacgtcagaacacgtggtacaacccaaagctacctcatcagtgatggcggcccccagcactgacggagccatgaatctcattaaaaacaaacgaaacatgactggttacaagactcttccttaccctctgaagaaacagaatggcaagatcaagtatgagtgcaatgtctgtgccaagacgttcggtcagctctccaacctgaaggtccacctgagagtgcacagtggagaacggcctttcaagtgccagacctgcaacaagggttttactcagctcgcccacctgcagaaacactacttggtacacacaggagagaagccacatgagtgccaggtctgccacaagagatttagcagcacaagcaatctcaagacccaccttcgattgcattctggagaaaaaccttaccaatgtaaggtgtgccctgccaagtttacgcaatttgtgcacctgaagctgcacaagcgactgcatacccgggagcggcctcacaagtgtgcccagtgtcacaagagctacatccatctctgcagcctcaaggtccacctgaagggcaactgccctgcgggcccagctgctgggctgcctttggaggatctgacccgaatcaatgaagaaattgagaggttcgacatcagcgacaatgcagaccgtcttgaggacatggaggacagtgtcgatgtgacctccatggtggagaaggagattctagctgtggtcagaaaagagaaagaagaaaccagtctgaaagtgtctttgcaaagaaacatggggaacggcctcctctcctcagggtgcagcctctatgagtcatcggacctgtccctcatgaagttgcctcacagcaacccactacctctggtgcctgtaaaggtcaaacaagaaacagttgaaccgatggatccttaagattttcagaaaataagtgtttcgtgttgcttcttagggtatggcttggtgaatcagggtgcctttagcaaattgcttgtacatgactccagatctgcaaagctccgctggcaccgggtgcttccctgcacctctctggaattaaagaaggactccaatgttaccaaaatctcagggcataaatgaggcaaagactcactatatatacatatatacatatatacatattataaatatatatatacttatttacagccatgtctatatatttgaacctgtgtattttgaatatttgtgtggatatgtttgcatagcgccttcctattactaaaactattgcctagccataattattttttcaatgataattcttcataatttattatacagtttatctttcaaaaagcaataattaaagaagtttacaatgactggaaagattctttgtaatttgagtataaatgttgtatctttgtcctgtggccattctttgtagataatttctgcacatctgtttaaatgcctgagacttagaagatagctctgtgatttcaggcaacctttctctatgataatgctttaaaatgaggttttgatattgccaaagtcatgtggttggtgtgttaactcagaagatcacacaatctgagtgacattctctaagttggggatacatgtgcagaattgctcagcaataatttgaggggaaggaagaagaaaaatattttatgtttcagaatgatggtttggttttcctcctcctagtcacaattttaccaaacagtgacaggaaggctttgccaacctgtctcccaatgtcacatgaccattctgagtggccatatgactttggcatccctgggtgttatctgaaaatgtgaagaagataaaaaagccgtgttcagaagatctgtcgtaaagcacagatgttgtgtgtgtgtgtgtgtgggttggggggtttgagtctggctgtcattttgctgttggcttgtttttgtttttttaatatcaaaattgcacaaagctggtgccctaccaagaaggatttgatatagaaaggctcaggccacacttaaaatacaagcaagcaaagagaacagaaaaaaataaaagtaaaaacgggtattcttatcatcttaggttaagcgggtaatgaacattcctgtccccaacgcatcaactgtattgtatctgtaaaactcagcttttctcagtatttgtgtttttgcattgtataattaacttaattaaagatgaaagggcattgcaaaagtgttcaacaattacctcattgagtgtatccagtaggagtgcaggaattaatgtcgtatctcatgagttgctacccagctgagcgtgtgtgcttccaaatggtaggctgggtggttcggtcctgtattctcctaagcccaaaggttacctgttggtgttcaaggtgtaataaagaatgctgtatatttatgaacctatttataccagtataccatgtgtatatatgatatatttataaccacttaaattgtgagccaagccatgtaaaagaacctatttttcctaagagcaaaaagaatctctctgaagttttgcttaaaactccatgacctcgctatgactttggtgcttgggcaccaccctgcctactaccagagagcagagcacctcagtgcagaggtgagggtgtgtagcatcttgggatggatagaaacaccacaccatccagtcgcatttgatggccttgctacatgtgtgtcagttgggtcacagaataaaaatcatttttctatttctgctctcctcttcctcttcctcttcttcttcctctttctctccctcctctagaaccctgactcatgctcactgctcagtctgatgcttaccttagagttttgtatatatagatcaacttacaaagagggaaaacttcagatcctctgggggaaacccaagagccttactgacctgttgctgtgactagctagatgggtttctctttaccttccaaggatcaaaaccagagattccacacatgctagcaagcaagcaagctgtcactgggctgcagccccaacaagactgacatttctggatgcatctgtatttgagaaaaatactcacttaattgtaggttaaccaaagcatgacctgacattgacaccaaatacaaatacgatttctttgcagtgaacttgggttgttttcctcctgtgcttttcttgtgttgggaggatttttacaaggacaattgcttttcttgccatctgtcttcctcttaggcctcttacatgagagtgttgagcccacaatgaacagtggttggttggttggttggttggtttgtttgtttgttttttcctcagagttaaaactgaccaaagttattggctttttactttgctagaacaacaaactatcttatgtttacgtactggtttacattgttatttatgtgcaaattgtcaaaatgtaaattaaaatataaatgttcatgctttaaaaaaaaaaaaaaaaa

SEQ ID NO: 17 - DNA, 2472 bp, Mus musculus
  misc_feature (1)..(2472): PRDM1 Mouse CDS >NM_007548.4241-2712 Mus musculus PR domain containing 1, with ZNF domain (Prdm1), mRNA
atgttggatcttctcttggaaaaacgtgtgggtacgaccttggctgcccccaagtctagctccggctccgtgaagtttcaaggactggcagagactgggatcatgaaaatggacatggaggacgctgatatgactttgtggacagaggccgagtttgaagagaagtgtacatacatagtgaacgaccacccctgggattccggcgctgacgggggtacttctgttcaagccgaggcatccttaccaaggaacctgcttttcaagtatgctgccaacaacagcaaagaggttattggcgtggtaagtaaggagtacataccgaagggaacacgctttggacccctcatcggtgaagtctacactaatgacacagttcccaagaatgccaacaggaagtatttttggcggatctattccagagaggagttccaccacttcattgatggctttaatgaggagaaaagcaactggatgcgctacgtgaatccagctcactctgcccgggagcaaaacctggctgcctgtcagaacgggatgaacatctacttctacactattaagcctatccctgccaaccaggaacttcttgtgtggtattgtcgggactttgcggagaggctccactacccttatcctggagagctcacagtgataaatctcacacaaacggaaagcaacccaaagcaatacagtagtgagaaaaatgaactctacccaaagagtgtccccaagagagagtacagcgtgaaagaaattctaaaactggactccaatccctccaaaaggaaggacatctaccgttccaacatttcacccttcactttagaaaaggacatggatggctttcggaaaaatgggagccccgacatgcccttctaccctcgggtggtttatcctatccgggcacctctgccagaagactttttgaaagcgtccctggcctatgggatggagagacccacctacataactcacagtccccttccgtcttccacaactccaagtccccctgcgagcagcagcccggagcagagccttaagagctccagcccccacagcagcccgggaaacacggtgtcacccctggcgccaggcctcccagaacaccgggactcctactcctacttgaatgtttcctatggttccgagggcctgggctcctaccctggctatgcacctgccccccacctcccaccagctttcattccttcttacaatgctcactaccccaagttcctgttgccaccgtacggcattagttccaatggcttgagcaccatgaacaacatcaatggtatcaacaacttcagcctcttccctaggttgtatcccgtctacagtaacctccttagtggcagcagcctgcctcatcccatgctcaatccagcttccctaccgagttccctgcctaccgatggagcccggaggctgcttccaccggagcaccccaaagaggtgcttatcccagcaccccacagtgccttctcccttaccggggctgcagccagcatgaaggacgagagtagtccccccagcggatctccaacggcgggaactgcagccacgtcagaacacgtggtacaacccaaagctacctcatcagtgatggcggcccccagcactgacggagccatgaatctcattaaaaacaaacgaaacatgactggttacaagactcttccttaccctctgaagaaacagaatggcaagatcaagtatgagtgcaatgtctgtgccaagacgttcggtcagctctccaacctgaaggtccacctgagagtgcacagtggagaacggcctttcaagtgccagacctgcaacaagggttttactcagctcgcccacctgcagaaacactacttggtacacacaggagagaagccacatgagtgccaggtctgccacaagagatttagcagcacaagcaatctcaagacccaccttcgattgcattctggagaaaaaccttaccaatgtaaggtgtgccctgccaagtttacgcaatttgtgcacctgaagctgcacaagcgactgcatacccgggagcggcctcacaagtgtgcccagtgtcacaagagctacatccatctctgcagcctcaaggtccacctgaagggcaactgccctgcgggcccagctgctgggctgcctttggaggatctgacccgaatcaatgaagaaattgagaggttcgacatcagcgacaatgcagaccgtcttgaggacatggaggacagtgtcgatgtgacctccatggtggagaaggagattctagctgtggtcagaaaagagaaagaagaaaccagtctgaaagtgtctttgcaaagaaacatggggaacggcctcctctcctcagggtgcagcctctatgagtcatcggacctgtccctcatgaagttgcctcacagcaacccactacctctggtgcctgtaaaggtcaaacaagaaacagttgaaccgatggatccttaa

SEQ ID NO: 18 - protein, 823 aa, Mus musculus
  MISC_FEATURE (1)..(823): PRDM1 Mouse Protein NP_031574.2 PR domain zinc finger protein 1 [Mus musculus]
MLDLLLEKRVGTTLAAPKSSSGSVKFQGLAETGIMKMDMEDADMTLWTEAEFEEKCTYIVNDHPWDSGADGGTSVQAEASLPRNLLFKYAANNSKEVIGVVSKEYIPKGTRFGPLIGEVYTNDTVPKNANRKYFWRIYSREEFHHFIDGFNEEKSNWMRYVNPAHSAREQNLAACQNGMNIYFYTIKPIPANQELLVWYCRDFAERLHYPYPGELTVINLTQTESNPKQYSSEKNELYPKSVPKREYSVKEILKLDSNPSKRKDIYRSNISPFTLEKDMDGFRKNGSPDMPFYPRVVYPIRAPLPEDFLKASLAYGMERPTYITHSPLPSSTTPSPPASSSPEQSLKSSSPHSSPGNTVSPLAPGLPEHRDSYSYLNVSYGSEGLGSYPGYAPAPHLPPAFIPSYNAHYPKFLLPPYGISSNGLSTMNNINGINNFSLFPRLYPVYSNLLSGSSLPHPMLNPASLPSSLPTDGARRLLPPEHPKEVLIPAPHSAFSLTGAAASMKDESSPPSGSPTAGTAATSEHVVQPKATSSVMAAPSTDGAMNLIKNKRNMTGYKTLPYPLKKQNGKIKYECNVCAKTFGQLSNLKVHLRVHSGERPFKCQTCNKGFTQLAHLQKHYLVHTGEKPHECQVCHKRFSSTSNLKTHLRLHSGEKPYQCKVCPAKFTQFVHLKLHKRLHTRERPHKCAQCHKSYIHLCSLKVHLKGNCPAGPAAGLPLEDLTRINEEIERFDISDNADRLEDMEDSVDVTSMVEKEILAVVRKEKEETSLKVSLQRNMGNGLLSSGCSLYESSDLSLMKLPHSNPLPLVPVKVKQETVEPMDP

SEQ ID NO: 19 - DNA, 2257 bp, Mus musculus
  misc_feature (1)..(2257): IRF2 Human - Full Sequence >NM_002199.4 Homo sapiens interferon regulatory factor 2 (IRF2), mRNA
ctctccttgttttgctttcgatctggactgttctcaggcaagccggggagtaacttttagttttgctcctgcgattattcaactgacgggctttcatttccatttcacataccctagcaacacttataccttgcggaattgtattggtagcgtgaaaaaagcacactgagagggcaccatgccggtggaaaggatgcgcatgcgcccgtggctggaggagcagataaactccaacacgatcccggggctcaagtggcttaacaaggaaaagaagatttttcagatcccctggatgcatgcggctagacatgggtgggatgtggaaaaagatgcaccactctttagaaactgggcaatccatacaggaaagcatcaaccaggagtagataaacctgatcccaaaacatggaaggcgaatttcagatgcgccatgaattccttgcctgatattgaagaagtcaaggataaaagcataaagaaaggaaataatgccttcagggtctaccgaatgctgcccctatcagaacggccttctaagaaaggaaagaaaccaaagacagaaaaagaagacaaagttaagcacatcaagcaagaaccagttgagtcatctctggggcttagtaatggagtaagtgatctttctcctgagtatgcggtcctgacttcaactataaaaaatgaagtggatagtacggtgaacatcatagttgtaggacagtcccatctggacagcaacattgagaatcaagagattgtcaccaatccgccagacatttgccaagttgtagaggtgaccactgagagcgacgagcagccggtcagcatgagcgagctctaccctctgcagatctcccccgtgtcttcctatgcagaaagcgaaacgactgatagtgtgcccagcgatgaagagagtgccgaggggcggccacactggcggaagaggaatattgaaggcaaacagtacctcagcaacatggggactcgaggctcctacctgctgcccggcatggcgtccttcgtcacttccaacaaaccggacctccaggtcaccatcaaagaggagagcaatccggtgccttacaacagctcctggcccccttttcaagacctccccctttcttcctccatgaccccagcatccagcagcagtcggccagaccgggagacccgggccagcgtcatcaagaaaacatcggatatcacccaggcccgcgtcaagagctgttaagcctctgactctccgcggtggttgttggggcttcttggctttgttttgttgtttgtttgtattttatttttttctctctgacacctattttagacaaatctaagggaaaaagccttgacaatagaacattgattgctgtgtccaactccagtactggagcttctctttaactcaggactccagcccattggtagacgtgtgtttctagagcctgctggatctcccagggctactcactcaagttcaaggaccaacaagggcagtggaggtgctgcattgcctgcggtcaaggccagcaaggtggagtggatgcctcagaacggacgagataatgtgaactagctggaattttttattcttgtgaatatgtacataggcagcactagcgacattgcagtctgcttctgcaccttatcttaaagcacttacagataggccttcttgtgatcttgctctatctcacagcacactcagcacccccttctctgcccattccccagcctctcttcctatcccatcccatcccatcccatcccatcccatcccatcccgctcttttcctacttttccttccctcaaagcttccattccacatccggaggagaagaaggaaatgaatttctctacagatgtcccattttcagactgctttaaaaaaaatccttctaatctgctatgcttgaatgccacgcggtacaaaggaaaaagtatcatggaaatattatgcaaattcccagatttgaagacaaaaatactctaattctaaccagagcaagcttttttattttttatacaggggaatattttattcaaggtaaaattctaaataaaatataattgttttttatcttttctacagcaaatttataattttaagattccttttcttgtttatcagcagttgttattacatccttgtggcacatttttttttttaattttgtaaaggtgaaaaaagcttttatgagctcatgtagcaatcagattttcctgtggattgataataaatgaatatgatatatagttaaa

SEQ ID NO: 20 - DNA, 1050 bp, Homo sapiens
  misc_feature (1)..(1050): IRF2 Human CDS >NM_002199.4179-1228 Homo sapiens interferon regulatory factor 2 (IRF2), mRNA
atgccggtggaaaggatgcgcatgcgcccgtggctggaggagcagataaactccaacacgatcccggggctcaagtggcttaacaaggaaaagaagatttttcagatcccctggatgcatgcggctagacatgggtgggatgtggaaaaagatgcaccactctttagaaactgggcaatccatacaggaaagcatcaaccaggagtagataaacctgatcccaaaacatggaaggcgaatttcagatgcgccatgaattccttgcctgatattgaagaagtcaaggataaaagcataaagaaaggaaataatgccttcagggtctaccgaatgctgcccctatcagaacggccttctaagaaaggaaagaaaccaaagacagaaaaagaagacaaagttaagcacatcaagcaagaaccagttgagtcatctctggggcttagtaatggagtaagtgatctttctcctgagtatgcggtcctgacttcaactataaaaaatgaagtggatagtacggtgaacatcatagttgtaggacagtcccatctggacagcaacattgagaatcaagagattgtcaccaatccgccagacatttgccaagttgtagaggtgaccactgagagcgacgagcagccggtcagcatgagcgagctctaccctctgcagatctcccccgtgtcttcctatgcagaaagcgaaacgactgatagtgtgcccagcgatgaagagagtgccgaggggcggccacactggcggaagaggaatattgaaggcaaacagtacctcagcaacatggggactcgaggctcctacctgctgcccggcatggcgtccttcgtcacttccaacaaaccggacctccaggtcaccatcaaagaggagagcaatccggtgccttacaacagctcctggcccccttttcaagacctccccctttcttcctccatgaccccagcatccagcagcagtcggccagaccgggagacccgggccagcgtcatcaagaaaacatcggatatcacccaggcccgcgtcaagagctgttaa

SEQ ID NO: 21 - protein, 349 aa, Homo sapiens
  MISC_FEATURE (1)..(349): IRF2 Human Protein >NP_002190.2 interferon regulatory factor 2 [Homo sapiens]
MPVERMRMRPWLEEQINSNTIPGLKWLNKEKKIFQIPWMHAARHGWDVEKDAPLFRNWAIHTGKHQPGVDKPDPKTWKANFRCAMNSLPDIEEVKDKSIKKGNNAFRVYRMLPLSERPSKKGKKPKTEKEDKVKHIKQEPVESSLGLSNGVSDLSPEYAVLTSTIKNEVDSTVNIIVVGQSHLDSNIENQEIVTNPPDICQVVEVTTESDEQPVSMSELYPLQISPVSSYAESETTDSVPSDEESAEGRPHWRKRNIEGKQYLSNMGTRGSYLLPGMASFVTSNKPDLQVTIKEESNPVPYNSSWPPFQDLPLSSSMTPASSSSRPDRETRASVIKKTSDITQARVKSC

SEQ ID NO: 22 - DNA, 2484 bp, Mus musculus
  misc_feature (1)..(2484): IRF2 Mouse - Full Sequence >NM_008391.4 Mus musculus interferon regulatory factor 2 (Irf2), transcript variant 1, mRNA
gctcctcgcagtttcctctccttgttttgctttcgatctggactgttctcaggcaagccggggactaacttttagttttgctcctgcgattattcaactgacgggctttcatttccattttacacaccctaacaacactcacaccttgcgggattgtattggtagcgtggaaaaaaaaaagcacattgagagggtaccatgccggtggaacggatgcgaatgcgcccgtggctggaggagcagataaattccaatacgataccagggctaaagtggctgaacaaggagaagaagattttccagatcccctggatgcatgcggctcggcacggatgggacgtggaaaaggatgctccgctcttcagaaactgggcgatccatacaggaaagcatcaaccaggaatagataaaccagatccaaaaacatggaaagcaaattttcgatgtgccatgaattccctgcccgacattgaggaagtgaaggacagaagcataaagaaaggaaacaacgccttcagagtctaccggatgctgcccttatccgaacgaccttccaagaaaggaaagaaaccaaagacagaaaaagaagagagagttaagcacatcaagcaagaaccagttgagtcatctttggggcttagtaatggagtaagtggcttttctcctgagtatgcggtcctgacttcagctataaaaaatgaagtggatagtacggtgaacatcatagttgtaggacagtcccatctggacagcaacattgaagatcaagagatcgtcactaacccgccagacatctgccaggttgtagaagtgaccactgagagtgatgaccagccagtcagcatgagtgagctctaccctctacagatttctcctgtgtcttcctacgcagaaagcgaaactaccgacagtgtggccagtgatgaagagaacgcagaggggagaccacactggaggaagaggagcatcgaaggcaagcagtacctcagcaacatggggacacggaacacctatctgctgcccagcatggcgacctttgtcacctccaacaagccagatctgcaggtcaccatcaaagaggatagctgtccgatgccttacaacagctcctggcccccatttacagaccttccccttcctgccccagtgacccccacgcccagcagcagtcggccagaccgggagacccgggccagtgtcatcaagaagacatctgatatcacccaggcccgtgtcaagagctgttaagcctttgactctccctggtggttgttgggatttcttagctttgtgttgttctttgtttgtattatattattttttttctctatgatacctatcttagacacatctaagggagaaagccttgacgatagattattgattgctgtgtccaactccagagctggagcttcttcttaactcaggactccagccccccccccccctcggtagatgcgtatctctagaacctgctggatctgccagggctactccctcaagttcaaggaccaacagccacacgggcagtggaggtgctgcgttgcctacggtcaaggccagcatggtggagtggatgcctcagaacggaggagaaaatgtgaactagctggaatttttttattcttgtgaatatgtacatagggcagtacgagcaatgtcgcgggctgcttctgcaccttatcttgaagcacttacaataggccttcttgtaatcttgctctccttcacagcacactcggcgaccccttctgtgtccactaccccactacccacccctccctcctcaacccctccatcccggtcctctatgcgccccttccccccaaccaatcccatcacaacctcttacctatcctttccctcccaaccccttctatcccagcccaccacctaccccactcctccccaactcctccattctagcccattacccacgcctctctcctcagcccagcctaccccatcccaccctgttcctttcctccagtttcctctcctcaaaggcaaggctctacatcttggaggaggaggaggagaagaaaatgagtttcttcaccgctgtcccattttaagactgcttgaataataaaaaaaaaatctttctaatctgctatgcttgaatggcacgcggtacaaaggaaaactgtcatggaaatattatgcaaattcccagatctgaagacggaaaatactctaattctaaccagagcaagcttttttatttttttatacaaggggaatattttattcaaggtaaaaaaattctaaataaaatataattgttttttatcttttctacagcaaatttataattttaagattccttttcctgttcatcagcagttgttattacatcccttgtggcacattttttttttaattttgtaaaggtgaaaaaaaaacttttatgagctcatgtagcaatcaaattatcctgtggattgataataaatgaatatggtatatagttaaagattttaa

SEQ ID NO: 23 - DNA, 1050 bp, Mus musculus
  misc_feature (1)..(1050)
atgccggtggaacggatgcgaatgcgcccgtggctggaggagcagataaattccaatacgataccagggctaaagtggctgaacaaggagaagaagattttccagatcccctggatgcatgcggctcggcacggatgggacgtggaaaaggatgctccgctcttcagaaactgggcgatccatacaggaaagcatcaaccaggaatagataaaccagatccaaaaacatggaaagcaaattttcgatgtgccatgaattccctgcccgacattgaggaagtgaaggacagaagcataaagaaaggaaacaacgccttcagagtctaccggatgctgcccttatccgaacgaccttccaagaaaggaaagaaaccaaagacagaaaaagaagagagagttaagcacatcaagcaagaaccagttgagtcatctttggggcttagtaatggagtaagtggcttttctcctgagtatgcggtcctgacttcagctataaaaaatgaagtggatagtacggtgaacatcatagttgtaggacagtcccatctggacagcaacattgaagatcaagagatcgtcactaacccgccagacatctgccaggttgtagaagtgaccactgagagtgatgaccagccagtcagcatgagtgagctctaccctctacagatttctcctgtgtcttcctacgcagaaagcgaaactaccgacagtgtggccagtgatgaagagaacgcagaggggagaccacactggaggaagaggagcatcgaaggcaagcagtacctcagcaacatggggacacggaacacctatctgctgcccagcatggcgacctttgtcacctccaacaagccagatctgcaggtcaccatcaaagaggatagctgtccgatgccttacaacagctcctggcccccatttacagaccttccccttcctgccccagtgacccccacgcccagcagcagtcggccagaccgggagacccgggccagtgtcatcaagaagacatctgatatcacccaggcccgtgtcaagagctgttaa

SEQ ID NO: 24 - protein, 349 aa, Mus musculus
  MISC_FEATURE (1)..(349): IRF2 Mouse Protein >NP_032417.3 interferon regulatory factor 2 [Mus musculus]
MPVERMRMRPWLEEQINSNTIPGLKWLNKEKKIFQIPWMHAARHGWDVEKDAPLFRNWAIHTGKHQPGIDKPDPKTWKANFRCAMNSLPDIEEVKDRSIKKGNNAFRVYRMLPLSERPSKKGKKPKTEKEERVKHIKQEPVESSLGLSNGVSGFSPEYAVLTSAIKNEVDSTVNIIVVGQSHLDSNIEDQEIVTNPPDICQVVEVTTESDDQPVSMSELYPLQISPVSSYAESETTDSVASDEENAEGRPHWRKRSIEGKQYLSNMGTRNTYLLPSMATFVTSNKPDLQVTIKEDSCPMPYNSSWPPFTDLPLPAPVTPTPSSSRPDRETRASVIKKTSDITQARVKSC

SEQ ID NO: 25 - DNA, 6720 bp, Homo sapiens
  misc_feature (1)..(6720): POU2F2 Human - Full Sequence >NM_001247994.2 Homo sapiens POU class 2 homeobox 2 (POU2F2), transcript variant 4, mRNA
acagttgttcccccagccctggcggggcgggcagcatggttcactccagcatgggggctccagaaataagaatgtctaagcccctggaggccgagaagcaaggtctggactccccatcagagcacacagacaccgaaagaaatggaccagacactaatcatcagaacccccaaaataagacctccccattctccgtgtccccaactggccccagtacaaagatcaaggctgaagaccccagtggcgattcagccccagcagcacccctgccccctcagccggcccagcctcatctgccccaggcccaactcatgttgacgggcagccagctagctggggacatacagcagctcctccagctccagcagctggtgcttgtgccaggccaccacctccagccacctgctcagttcctgctaccgcaggcccagcagagccagccaggcctgctaccgacaccaaatctattccagctacctcagcaaacccagggagctcttctgacctcccagccccgggccgggcttcccacacagccccccaaatgcttggagccaccatcccaccccgaggagcccagtgatctggaggagctggagcaattcgcccgcaccttcaagcaacgccgcatcaagctgggcttcacgcagggtgatgtgggcctggccatgggcaagctctacggcaacgacttcagccagacgaccatttcccgcttcgaggccctcaacctgagcttcaagaacatgtgcaaactcaagcccctcctggagaagtggctcaacgatgcagagactatgtctgtggactcaagcctgcccagccccaaccagctgagcagccccagcctgggtttcgacggcctgcccggccggagacgcaagaagaggaccagcatcgagacaaacgtccgcttcgccttagagaagagttttctagcgaaccagaagcctacctcagaggagatcctgctgatcgccgagcagctgcacatggagaaggaagtgatccgcgtctggttctgcaaccggcgccagaaggagaaacgcatcaacccctgcagtgcggcccccatgctgcccagcccagggaagccggccagctacagcccccatatggtcacaccccaagggggcgcggggaccttaccgttgtcccaagcttccagcagtctgagcacaacagcacaaaccccagccctcaaggcagccactcggctatcggcttgtcaggcctgaaccccagcacgggccctggcctctggtggaaccctgccccttaccagccttgatggcagcgggaatctggtgctgggggcagccggtgcagccccggggagccctggcctggtgacctcgccgctcttcttgaatcatgctgggctgcccctgctcagcaccccgcctggtgtgggcctggtctcagcagcggctgcggctgtggcagcctccatctccagcaagtctcctggcctctcctcctcatcctcttcatcctcatcctcctcctcctccacttgcagcgagacggcagcacagacccctggaggtccaggggggcccgaggcagggtccaaacctgagtgagggccagccatgcctcccctcccattcctctggtccctgccttggtcccttgcctgggaagagggcgaggaggccagtggtggggacgcagagggtcctcagagcaggagtgacaagggaggaaagaccaaaaaaacaaccaaccaaaaaaaaaaaaaaaaaggaaagaaactaaccaacaaaagagaaaaccaaaaataatcacaacagaaaccagctgccccaaaggaaccagaggtgaaaaacaaacaaaaaaaaaccaaaaacaaaccaaaaaaaaaaaaaacccacaaaatcaaacaaaccaaaaaaccagtcgcgagccagacctcagcgtgctcaccctcactgctacgacgccaaataaaaaccccagccaggggcggagaagcctccagcaggtcagacctcatgccaccgagccctggctgtgggaccaacccccaaccctgcctcccccgtgggggctacagaaggaaaaagagaagatgccagcttcctaatcccagcccccagccctgggccagcgaagacagggcacagcctgggcagatgggctggggcttagcaccacccaccaaatgttcttttccagaaggtgaaagagaaagggcctgaacaaccttacaccaaatattcagtagcttcatccaaaggatgtacagaatttttagcattgtgctcaacagaatgtgtccctaccatgtgtccccttctcccctggcccccagctctccccacctgggcagggggtcttgctttaacctcctccctccccccagcaggggagagttcaagggaaggcctgcggacaacttttcatcccctgttcctccctcttttcccctttgaagggtgggctaggccattttgccaagttctagctctcacaagtccctcctcaaccctgtcaccctcctctgctctggaactgactccctccccagcctatgggaaggtggaaatttcaggcaagaggggatgaagacattcagttgagggcattcagttgtctttttccatcctgtctgtttccctgaaaaaaaaaaaaattcatattccagtgcctatccgtgggatccttcacgttctttgacattaaccagaaaccaaaaagagaactcacctcgcttttcccaccctgtgcccctctggtactggcgaatgcctctccctccccccacatgcacgcacgcacacaccccagtggtggggttcctcgagatggcatccccatcagggtccatgtggtggggacagtgccacagcctgtggtccccatctgagagggcgcggtggtggccaccactcccacaagacctccacaactcttggctggaccctgtgtttgaccagccccggacacgtctccactgacgacggacagaggagagacaccactgggagccaccctgccctcctgctattgtggaggccaacaaagttttgaaccgaaaaccaaaaaaacagaaacaaacaaacaataaatttaaactagaaaaaaaatttatatatatatataaacatatatatatatatatatataaaagggaaagaagatgaggactcttcaagaataagatggaaccgcgaggcggagccaagcccctgcaccggtggtccaggcccttgttccccaaggcggatggaaggacggatgcttctttctcttcacaaatacctcatggacgttgtctttgggaaagccggagggggcggctggggcagggcctgtccctcggccagggcagatggaagcgggtgggcggggctgagagagggtatcagggacaggggtgaagagccccaaactcccctcccaaatcaactgaaaaagctttaaaaaaagacaggaaaaaaaggagtaagaaagcaaaaagaacagatgaaggaaaacttaacaacttttgggtggtttgattcctcctttgatttctttttcggttctcttttgtctgacctctctcctcccctccttcctctctcctcccctccttcctctctcctctcctccttcctgtctcctctctctccctttgctctcctctcttctctctcaatcttgttctttctgtgtctctcctcaaaccaaagtgttgcagtgaaggacacgagccattgaaatcagggtggggcctgacagccgtagtgtggcccctgccccttgttgggcaggagcagagagagaggagagccctgagaccgcagggctttggcctgggcagcttccactttctgccggacactctgaggagaggcagatggagctccaggcctcagcgttctcttttttccttgtctcccttctcccacccgggaaatgaaactgttgggctgggcgacggaggcagcagagactcggccattgcaggggcctctgggtgccttgtccgggcagtggtgaagtagccgcccctcccaccccggcacgatggggcctggcacctcgtctacccaacctcaccggaatgtaagcatctccgctgaacgactccctgcccttaccctacctctgagtttgtccatgtttatttcctgaagagagaagggactggcaagagggcttgggccccagcccaagggtggagagggggccaagggctcctgaccaaataggaaggacatttgagcagagcaaaatgaaaggaattacaaccaaaaaccccctccgagaagacaggcagcatggaaggcatggtggagatgactcaaacaaaaactatgattctagaccaaaaaggaaaaaaaaaaaaaaaaaaggaaagaaaatccaggactcaccaccacccacttcatcctgcttcctccccatcaagtcccaccacctgggacctctccccaccccagtatgtgggagtggggtaaagaggagaggaggagcagggggcagggatggggcagatgccctgatgttggtggagggacccctgtgcagccgggggtgctggggaccctccccatccaaccctcacacctaggaaaagaaaaacaagaaaaaaaaattcctgggagggggaaaaataaccaaatcccaagctttaactacagctgtgaaaccaaatattgggaagggggtaggagggagggaggggcaggtgagccccaggtctccccctgggcccccctcccctgaggactcgagataaggcaccaaatacctcatagaactttaatgttaaaaaaaggaaaccaaatatcgttggctgggggccagccagggcaaggggctggggggctggagggagccagggttgggaaggtgatgggggaattcatgccccccacccccatctgccccttacactctggtcccccgtctcgactccgggaaacaaaactatctcatcgtttttattttgtggtctgtacagagcctgtgtccgtgtgtctgtgtatgagcgagagagttggagggctggggagctttatgtgggggatggggagggcaaccccaggctaggctatgggctaggttagatgtccgaggttgggggccaagggcctgggctagaaagagaggagagatgagtgggttggagcagtggggagccctctgaatttctcttctccccaaccctgacctcctacttaagggagggacagagactgggtctaccctaatggtgggccttttcattgtgccaaaaaaaaaaaaaaaaaaattgatgccagtaactaaaccacctctctctgttctcttctggggggtggggtgggtgtgtgtgtgggggtgggtagggatggggaacccaagccccagtaggagttggggctgagatcaggaggaggggaaggatatgagttttccatccatctccctggagaaggccatgacgggctctccaagccctggggagatctggcgagggactagccaagtcagggtgacctcttgacctgactagtttttctaacccacatgaacaccagggtgcctctctcttcctctagcccagggtagggggacctgagtgagagagagagtgagacagacagcaagattgagacacgcgggcccccactggtctctgagtgggaaaggcaagtgcgagagataaaggcctccaagagaaaaaagaaacaaaccaaagatttaaccatttaaaaaaaaaaaaaaacccacagacaactccgctacctttttatatgttggaaaaaaagtgaaaaaaaattaaaaataaaaataaaaaaaaatacacaaaaactccaagccgcaagtccttcatgcggtttaaactttgacctcagcagtctccaaagcaagacgacgatgacaaaggcggaaacaaagaaaaaataatgtatatttttaagtatttgtccttgaattgttagctccttgttaaacaaacaaaaggagagaaaaatccagagagaagtgttgctgggatggagacaactatttactatgtctgtgacccctcccttctgcctccctccccttcctctctttcctgtcctctatccttgctgcccctcccccaatatgtcccccaaacccaggggctcagtatttatttatttatataattgcagggtgactgggggcctctctcggcaactcgtagagagcctttggataatggcagggtggggaatgggcgaggagtctttgggaagagcaggggcctgaactttcaactcctttctggcctcttgtcccactgcttccaccatcaccttccaaatccagggctgcactagggcaaggtgaccaccctacttggtaaagacctgattggcgcagcccattctggtgtgggtgtctttcaccacccagaatgaccaggttggaatgggggtgggtgaagttggagggggtggcaagtacaaggcaagggtgcccaggaaccggcatccagaaatcagccctggagtagacctgcctttcctccatccttaccaagttagcctccctttcagttccgtctgacccctgtgttggtagccccttccttcccttcctgtaccccctccgtctcttctcgtggtagcgggttagtgtttcagtgttcttaactccaatctgcttgttcattgtacaatgtgcttcttttaaggccccatttttgtaacttgagtgtgtcattcatctgaacaacaaacatcaaaaaataaaaaattaaaaactgta

SEQ ID NO: 26 - DNA, 1203 bp, Homo sapiens
  misc_feature (1)..(1203): POU2F2 Human CDS NM_001247994.236-1238 Homo sapiens POU class 2 homeobox 2 (POU2F2), transcript variant 4, mRNA
atggttcactccagcatgggggctccagaaataagaatgtctaagcccctggaggccgagaagcaaggtctggactccccatcagagcacacagacaccgaaagaaatggaccagacactaatcatcagaacccccaaaataagacctccccattctccgtgtccccaactggccccagtacaaagatcaaggctgaagaccccagtggcgattcagccccagcagcacccctgccccctcagccggcccagcctcatctgccccaggcccaactcatgttgacgggcagccagctagctggggacatacagcagctcctccagctccagcagctggtgcttgtgccaggccaccacctccagccacctgctcagttcctgctaccgcaggcccagcagagccagccaggcctgctaccgacaccaaatctattccagctacctcagcaaacccagggagctcttctgacctcccagccccgggccgggcttcccacacagccccccaaatgcttggagccaccatcccaccccgaggagcccagtgatctggaggagctggagcaattcgcccgcaccttcaagcaacgccgcatcaagctgggcttcacgcagggtgatgtgggcctggccatgggcaagctctacggcaacgacttcagccagacgaccatttcccgcttcgaggccctcaacctgagcttcaagaacatgtgcaaactcaagcccctcctggagaagtggctcaacgatgcagagactatgtctgtggactcaagcctgcccagccccaaccagctgagcagccccagcctgggtttcgacggcctgcccggccggagacgcaagaagaggaccagcatcgagacaaacgtccgcttcgccttagagaagagttttctagcgaaccagaagcctacctcagaggagatcctgctgatcgccgagcagctgcacatggagaaggaagtgatccgcgtctggttctgcaaccggcgccagaaggagaaacgcatcaacccctgcagtgcggcccccatgctgcccagcccagggaagccggccagctacagcccccatatggtcacaccccaagggggcgcggggaccttaccgttgtcccaagcttccagcagtctgagcacaacagcacaaaccccagccctcaaggcagccactcggctatcggcttgtcaggcctga

SEQ ID NO: 27 - protein, 400 aa, Homo sapiens
  MISC_FEATURE (1)..(400): POU2F2 Human Protein >NP_001234923.1 POU domain, class 2, transcription factor 2 isoform 4 [Homo sapiens]
MVHSSMGAPEIRMSKPLEAEKQGLDSPSEHTDTERNGPDTNHQNPQNKTSPFSVSPTGPSTKIKAEDPSGDSAPAAPLPPQPAQPHLPQAQLMLTGSQLAGDIQQLLQLQQLVLVPGHHLQPPAQFLLPQAQQSQPGLLPTPNLFQLPQQTQGALLTSQPRAGLPTQPPKCLEPPSHPEEPSDLEELEQFARTFKQRRIKLGFTQGDVGLAMGKLYGNDFSQTTISRFEALNLSFKNMCKLKPLLEKWLNDAETMSVDSSLPSPNQLSSPSLGFDGLPGRRRKKRTSIETNVRFALEKSFLANQKPTSEEILLIAEQLHMEKEVIRVWFCNRRQKEKRINPCSAAPMLPSPGKPASYSPHMVTPQGGAGTLPLSQASSSLSTTAQTPALKAATRLSACQA

SEQ ID NO: 28 - DNA, 3323 bp, Mus musculus
  misc_feature (1)..(3323): POU2F2 Mouse - Full Sequence >NM_001163554.1 Mus musculus POU domain, class 2, transcription factor 2 (Pou2f2), transcript variant 4, mRNA
cgggcagcatggttcattccagcatgggggctccagaaataagaatgtctaagcccctggaggccgagaagcaaagtctggactccccgtcagagcacacagacaccgaaagaaatggacccgacattaaccatcagaacccccagaataaagcgtccccattctctgtgtccccaactggccccagcaccaagatcaaggctgaagaccccagtggcgattcagccccagcagcacccccgcccccccagccggctcagcctcatctgccccaggcccaactcatgctgacgggcagccagctagctggggacatacagcaactcctccagctccagcagctggtgcttgtccccggccaccacctccagccacctgctcagttcctgctgccacaggcacagcagagtcagccaggcctgctaccaacgccaaatctattccagctacctcaacaaacccagggagctctcctgacctcccagccccgggctgggcttcctacacagcccccgaaatgcttggagccgccctcccacccggaggagcccagcgatctggaggagctggaacagtttgctcgcaccttcaagcaacgccgcatcaagctgggcttcacacagggtgatgtgggcctggccatgggcaagctctatggcaacgacttcagccaaacgaccatttcccgcttcgaggccctcaacctgagcttcaagaacatgtgtaaactcaagcccctcctggagaagtggctcaacgacgcagagactatgtctgtggattcaagcctacccagcccaaaccagctgagcagccccagcctgggtttcgacgggctgccggggcggagacgcaagaagaggaccagcatcgagacgaatgtccgcttcgccttagagaagagtttcctagcgaaccagaagcctacctcagaggagatcctgctgatcgcagagcagctgcacatggagaaggaagtgatccgcgtctggttctgcaaccggcgccagaaggagaaacgcatcaacccttgcagtgcggcccccatgctgcccagcccgggaaagccgaccagctacagccctcacctggtcacaccccaagggggcgcagggaccttaccattgtcccaagcttctagcagtctgagcacaacagttactaccttatcctcagctgtggggacgctccatcccagccggacagcaggagggggtgggggtgggggcggagctgcgccccccctcaattccatcccctctgtcactcccccacccccggccaccaccaacagcacaaacccgagccctcaaggcagccactcggctattggcttgtcgggcctgaaccccagcgcgggaagcacaatggtggggttgagctctgggctgagtccagccctcatgagcaacaaccctttggccactatccaagccctggcctctggtggaaccctgccccttaccagccttgatggcagcgggaacctggtgctgggggcagccggtgcggccccagggagtcccagcttagtaacctcgcctctcttcttgaaccacaccggtctgccgctgctcagtgccccaccaggcgtgggcctggtctcagcggcggctgcagccgtagcagcatccatctccagcaagtctcctggcctctcctcgtcttcttcatcctcatcatcctccacgtgcagtgatgtggcagcacagacccctggaggccccggaggacccgaggcggggtccaaggctgagtgagagccggccatgcctcccctcctactcctctgaatctcccgaccttggtcccctgcccaagagagggtaaggaggctggtgggggatacggtgggtccttggagcaggagtgacaaaggaggaaaagaccaaaaatccaaccaaaaagaaaagaaaagaaaaaacgacaaaacaaaaaaaataaataaataaacaaaaaaacaaagagaccaaccaacaaaaaagaaaaccaaaaatattcacaacagaaaccagctgccctaaaggaaacagatggaaaacaaagaaacgaacaactacaacaaaacaacaacaacaacaacaaaaaacacctccaaactaaccaaacacccccacccccaaaccaaacaaaccaaaaaccagtcttgagccagatcttggagtgctcactgctatgacaccaaataagaaccttcagccaggagcggagcagcctccagcaggtcggacctcatgccgcggagccctggctgtgggaccaactcctaaccttaccccactgagggtgggcagagaaggaaccagagaatgtgccagcttgctggcccagccccagctctgggccaatggagacaggtactgcctggggcaggaggcccagggcccagagccacctaccaaatggtcttttccacaaggtaaaagagaaaggacccgaacaaccttacaccaaatactcagtagcttcatccaaaggatgtacagagttttcagcgttgcgctcaacagaatgtgtgtcccttctacgtgtttcctccccccccagccccccagctctccccaacagggcagggggtctcgctttaacccctccccccgccacccccagtctggggagagttcagaggaaggcctgggaatgggcttttttgtcctctgcccctttctttctccctttgaagggtgggctaggccattttttgccgagttccagctctcaaaagcccctcctgagctcccccatcacctttctgctctggagtctacctcctccccagactgtgggaaggtggtcggttcaagcaaagggggatacagtgagggcaaccaagttgtctttcttcccatcctgtctgtcagtttccctgaaaaaaaaaatcatattccagtgcctatccgtgggatcttcacgttctttgacattaaccagaaaccaaaaagattaactcgcctcgctctgcccaccctacaccccctatgatactggcagatacctctcctgtccccttccctccccaccttccaatgcacacacgcacacaccccagtggtgggctcctccggatggcatctccatcagaaccacatggtggtgacatcactatgacctgtggttccccatgcaagtggctaccactcccacgagacctccacgagtattggctggaccctgtatttgcctagctctggacaaatctcaacagtggacagaggagatgctactgggagccaccatgccctcctaccatggaggccaacaaagttttgaaccaaaaaaaaaaaaccaaaccaaacaaacaaacacacacaaaacccaacaaa

SEQ ID NO: 29 - DNA, 1752 bp, Mus musculus
  misc_feature (1)..(1752): POU2F2 Mouse CDS >NM_001163554.19-1760 Mus musculus POU domain, class 2, transcription factor 2 (Pou2f2), transcript variant 4, mRNA
atggttcattccagcatgggggctccagaaataagaatgtctaagcccctggaggccgagaagcaaagtctggactccccgtcagagcacacagacaccgaaagaaatggacccgacattaaccatcagaacccccagaataaagcgtccccattctctgtgtccccaactggccccagcaccaagatcaaggctgaagaccccagtggcgattcagccccagcagcacccccgcccccccagccggctcagcctcatctgccccaggcccaactcatgctgacgggcagccagctagctggggacatacagcaactcctccagctccagcagctggtgcttgtccccggccaccacctccagccacctgctcagttcctgctgccacaggcacagcagagtcagccaggcctgctaccaacgccaaatctattccagctacctcaacaaacccagggagctctcctgacctcccagccccgggctgggcttcctacacagcccccgaaatgcttggagccgccctcccacccggaggagcccagcgatctggaggagctggaacagtttgctcgcaccttcaagcaacgccgcatcaagctgggcttcacacagggtgatgtgggcctggccatgggcaagctctatggcaacgacttcagccaaacgaccatttcccgcttcgaggccctcaacctgagcttcaagaacatgtgtaaactcaagcccctcctggagaagtggctcaacgacgcagagactatgtctgtggattcaagcctacccagcccaaaccagctgagcagccccagcctgggtttcgacgggctgccggggcggagacgcaagaagaggaccagcatcgagacgaatgtccgcttcgccttagagaagagtttcctagcgaaccagaagcctacctcagaggagatcctgctgatcgcagagcagctgcacatggagaaggaagtgatccgcgtctggttctgcaaccggcgccagaaggagaaacgcatcaacccttgcagtgcggcccccatgctgcccagcccgggaaagccgaccagctacagccctcacctggtcacaccccaagggggcgcagggaccttaccattgtcccaagcttctagcagtctgagcacaacagttactaccttatcctcagctgtggggacgctccatcccagccggacagcaggagggggtgggggtgggggcggagctgcgccccccctcaattccatcccctctgtcactcccccacccccggccaccaccaacagcacaaacccgagccctcaaggcagccactcggctattggcttgtcgggcctgaaccccagcgcgggaagcacaatggtggggttgagctctgggctgagtccagccctcatgagcaacaaccctttggccactatccaagccctggcctctggtggaaccctgccccttaccagccttgatggcagcgggaacctggtgctgggggcagccggtgcggccccagggagtcccagcttagtaacctcgcctctcttcttgaaccacaccggtctgccgctgctcagtgccccaccaggcgtgggcctggtctcagcggcggctgcagccgtagcagcatccatctccagcaagtctcctggcctctcctcgtcttcttcatcctcatcatcctccacgtgcagtgatgtggcagcacagacccctggaggccccggaggacccgaggcggggtccaaggctgagtga

SEQ ID NO: 30 - protein, 583 aa, Mus musculus
  MISC_FEATURE (1)..(583): POU2F2 Mouse Protein >NP_001157026.1 POU domain, class 2, transcription factor 2 isoform 4 [Mus musculus]
MVHSSMGAPEIRMSKPLEAEKQSLDSPSEHTDTERNGPDINHQNPQNKASPFSVSPTGPSTKIKAEDPSGDSAPAAPPPPQPAQPHLPQAQLMLTGSQLAGDIQQLLQLQQLVLVPGHHLQPPAQFLLPQAQQSQPGLLPTPNLFQLPQQTQGALLTSQPRAGLPTQPPKCLEPPSHPEEPSDLEELEQFARTFKQRRIKLGFTQGDVGLAMGKLYGNDFSQTTISRFEALNLSFKNMCKLKPLLEKWLNDAETMSVDSSLPSPNQLSSPSLGFDGLPGRRRKKRTSIETNVRFALEKSFLANQKPTSEEILLIAEQLHMEKEVIRVWFCNRRQKEKRINPCSAAPMLPSPGKPTSYSPHLVTPQGGAGTLPLSQASSSLSTTVTTLSSAVGTLHPSRTAGGGGGGGGAAPPLNSIPSVTPPPPATTNSTNPSPQGSHSAIGLSGLNPSAGSTMVGLSSGLSPALMSNNPLATIQALASGGTLPLTSLDGSGNLVLGAAGAAPGSPSLVTSPLFLNHTGLPLLSAPPGVGLVSAAAAAVAASISSKSPGLSSSSSSSSSSTCSDVAAQTPGGPGGPEAGSKAE

SEQ ID NO: 31 - DNA, 3631 bp, Homo sapiens
  misc_feature (1)..(3631): TGIF1 Human - Full Sequence >NM_170695.5 Homo sapiens TGFB induced factor homeobox 1 (TGIF1), transcript variant 1, mRNA
acattctttcagacccggcccgctgcggggcgttcctggggggtagcctcaaggccagcggggttccttcggctgcgtttctgtgggaggccctgaaacgcgcggagcttccctctgcctccaggctttcccagcgagagtgaaattaaacttgaaactcggatcaactggcagtcgttgttggtagaacgccctaaggacccctccccgcgggacggagggaggactcgggacagggaattggccctgggagaaaacgcgcggggggcgtccgagacgccccgtgaaagccgtgccgacccttgggaggactgacaggtctagagacacgcgctgtctgttgtggtgggcctcccgggaataagtgaggggctctgtgtttcgaggatggttctagcgcagagccgggtgtctgccggggtgggctccccgcattgttcgggctccggcgggggcggctctgattcctttccatggcccgcctcccaccccgggaatccccagtgctccttttccacggcttttctggcgtccccccgactctcccgcggcactttggcctaccttcccccagcgccgtggtcctccctggcgaccccctctgcgctcctggggtcctcctgcgccccccctcctccaccggcgcgctgcccacagccgcgtgccctctccccggagctggggaccaaggctgggccccgccggccgcatcggtgggaacttccgcggtccccatcccagggcgcacagggtccagctcctcggcgccgactcctggaaacaatgaaaggtattgttgcagcatctggcagtgagactgaggatgaggacagcatggacattcccttggacctttcttcatccgctggctcaggcaagagaaggagaaggggcaacctacccaaggagtctgtgcagattcttcgggattggctgtatgagcaccgttacaatgcctatccttcagagcaagaaaaagcgttgctgtcccagcaaacacacctgtctacgctacaggtctgtaactggttcatcaacgcccgccgcaggctcctccctgacatgctgagaaaggatggcaaagatccaaatcagttcacaatttcccgccgtggggccaagatttctgaaacgagctctgtggagtccgtgatgggcatcaaaaacttcatgccagctctagaggagaccccatttcattcctgtacagctgggccaaacccaaccctagggaggccactgtctcctaagccgtcatccccgggatcagttttggctcgtccatcagtgatctgccataccactgtgactgcattgaaagatgtccctttctctctctgccagtcggtcggtgtgggacaaaacacagatatacagcagatagcggccaaaaacttcacagacacctctctcatgtacccagaggacacttgtaaatctggaccaagtacgaatacacagagtggtcttttcaacactcctccccctactccaccggacctcaaccaggacttcagtggatttcagcttctagtggatgttgcactcaaacgggctgcagagatggagcttcaggcaaaacttacagcttaacccattttcaagcaaaacagttctcagaaatgtcatgattgccggggtgaaggcaagagatgaattgcattattttatatattttttattaatatttgcacatgggattgctaaaacagcttcctgttactgagatgtcttcaatggaatacagtcattccaagaactataaacttaaagctactgtagaaacaaagggttttcttttttaaatgtttcttggtagattattcataatgtgagatggttcccaatatcatgtgattttttttttcctccccttccctttttttgttattttttcagactgtgcaatacttagagaacctatagcatcttctcattcccatgtggaacaggatgcccacatactgtctaattaataaattttccattttttttcaaacaagtatgaatctagttggttgatgccttttttttcatgacataataaagtattttctttaaaaattgttgtaattcagagtatttctgttgagggaggtgcttcttaaaaataagtaggaatatagcaccccagtgagcaggaagctgggggggtagggtgcagtgttagggggtgtcaccagctctttgaaaacctgtggacaacaagccagtttgcataaacaggatgtgtgatatttactcttgataggaggcatagcaggcccttagagctttacttaaactgcatggcaaattgaaatgaatcattttagtgtttagctagccactaaatcccttgctgatctgtcctgcgtagtttaaaacgtggccatgttataaagaaaagtcttgaagcatccggttattgctgagttttcagaagaaaacaacgcaagcaatctcttgccctttttccacgttaccctttaaaataacctttgttcagtggtacatttagttaaggcatttgtattcaaatgtagcatagtatttgcagagaaattaccttttaaaacatctcaaaagtgttttctaaaatgatgaaataaacggtggggttctgtctatggtgggattgtcagtttattaggtgagtattttccaaggtctcagtttgtgaaaaaatggtgctatgtacaagtggagtttgttgggatagacaatcacttacaaagcccagatggccagaagggaattggtgttgctcctttcacttgtatggttgagcagatttgtcacatgccacaaaaccattctttgtagtcatgcttagtatgagcttgctttgttgttacggtcatgttctttccagccctgccccagctatgaattgtctaagtgtgagggagcatcagagcttcagtattgtttaaagaatgtagaaaaatacaaatttgtctaattttgaaactattttagaaagtccctactgtagaaagagctaaactcaaagactttccttgtaagtgaaagaaaaaaactgctgatgattaccaagaggcgtaagaaaattaggtgagggaatgttgtgctctagcctgtggggtgcagcttcgtagggcacatcagcttagaatggtctggcctcacctcagagagtcacagaaatagacaaaaacccaaaaagtactagtaaagtaaatacaaggaacaaaataaacattaataaaaatgaatgacgcaaaggtgatccagaatcattacttgctccaacagctagatcaaaggctgaacattttgctttagtatagatgagggaaatgagcaagtcctaaaaggtttgccctgttacggtgtttcatcactcatactggaaggagaaggaaaggagccatatgactcatttaaaagaaaaactgtaagtcaccttaaattccctatgttttacttcattttttccctattgaggttgttacaggtcatggttgataatagttggtgttgaatgaatgtaatccattatttaaaaacaggtacatttaagtgaaaagataaatgtaaaaagctgttcatatcaaagccaaatagaaatgatttttatgcttataatttacatggtgcaatatatctttttaatttgtttactttt

SEQ ID NO: 32 - DNA, 759 bp, Homo sapiens
  misc_feature (1)..(759): TGIF1 Human CDS >NM_170695.5835-1593 Homo sapiens TGFB induced factor homeobox 1 (TGIF1), transcript variant 1, mRNA
atggacattcccttggacctttcttcatccgctggctcaggcaagagaaggagaaggggcaacctacccaaggagtctgtgcagattcttcgggattggctgtatgagcaccgttacaatgcctatccttcagagcaagaaaaagcgttgctgtcccagcaaacacacctgtctacgctacaggtctgtaactggttcatcaacgcccgccgcaggctcctccctgacatgctgagaaaggatggcaaagatccaaatcagttcacaatttcccgccgtggggccaagatttctgaaacgagctctgtggagtccgtgatgggcatcaaaaacttcatgccagctctagaggagaccccatttcattcctgtacagctgggccaaacccaaccctagggaggccactgtctcctaagccgtcatccccgggatcagttttggctcgtccatcagtgatctgccataccactgtgactgcattgaaagatgtccctttctctctctgccagtcggtcggtgtgggacaaaacacagatatacagcagatagcggccaaaaacttcacagacacctctctcatgtacccagaggacacttgtaaatctggaccaagtacgaatacacagagtggtcttttcaacactcctccccctactccaccggacctcaaccaggacttcagtggatttcagcttctagtggatgttgcactcaaacgggctgcagagatggagcttcaggcaaaacttacagcttaa

SEQ ID NO: 33 - protein, 275 aa, Homo sapiens
  MISC_FEATURE (1)..(275): TGIF1 Human Protein >NP_001265611.1 homeobox protein TGIF1 isoform e [Homo sapiens]
MAAAHSGRSIVAASGSETEDEDSMDIPLDLSSSAGSGKRRRRGNLPKESVQILRDWLYEHRYNAYPSEQEKALLSQQTHLSTLQVCNWFINARRRLLPDMLRKDGKDPNQFTISRRGAKISETSSVESVMGIKNFMPALEETPFHSCTAGPNPTLGRPLSPKPSSPGSVLARPSVICHTTVTALKDVPFSLCQSVGVGQNTDIQQIAAKNFTDTSLMYPEDTCKSGPSTNTQSGLFNTPPPTPPDLNQDFSGFQLLVDVALKRAAEMELQAKLTA

SEQ ID NO: 34 - DNA, 1720 bp, Mus musculus
  misc_feature (1)..(1720): TGIF1 Mouse - Full Sequence >NM_001164075.1 Mus musculus TGFB-induced factor homeobox 1 (Tgif1), transcript variant 1, mRNA
gcgctgtgccgcgcccgcgtgtcaatggcagctgcgccttctggaagaagttgattcttgtctggaatacttttccttcggtgccccaccccttgaatcagaagctgctgcctccggcggctggagcagagacagtgaagtttggggcagctgtcaaaaacaaggatgggggtgtcttttcttctctggggtggacagaggagatgtccttaaacctctctccatcctttacaaactaaatgcactcttcgctagcccggggaggaaaggccccaaaagagaaaccagtgtggccgctgagagctgagggatggagatggtgctctcgccacgggcaggcaggcatgaccctgcttctgggttccgccagcgcgtagagggacgggtgatctcggcccccaggcccagggttgtggagctggaaggtcttgttgcagcatctggcagtgactctgaggatgaagacagcatggacagtcccctggacctttcctcatcagcagcctctggcaagagaaggaggagaggcaatctgcccaaggagtcagtccagattctgcgagactggctgtatgaacacagatacaacgcctatccctcagagcaagagaaagcactgctgtcccagcagacacacctgtccacactacaggtctgtaactggttcatcaacgcccgccgcaggctccttcctgacatgctgagaaaggatggcaaagatccaaatcagttcacgatttcccgccgtggggccaagatttcagaagctagctctattgaagctgcaatgggtatcaaaaacttcatgccaactctagaagagagcccatttcattcctgcgtagttggacccaacccaaccctagggagaccagtgtctcccaaacctccctccccaggatccattttggctcgcccgtcagtgatctgccataccactgtgactgcattgaaggatgggcctttctctctctgtcagccgattggtgtgggacagagtacagatgtaccgcaaatagcacccagcaactttacagacacctctctcgtgtacccagaggacacttgcaaatctggacccagtccaaaccctcagagtggtcttttcaacactcctccccctactccaccagacctcaaccaggattttagtggattccagcttctagtggatgttgcactcaaacgagcggcagagatggagcttcaggccaaactcacagcttaaccgttttttcaaacaaaacagttctccaaaatacggtcctgattgccgggggtgatggcaagagatgcattattttatatatttttctattaatatttgcacatgggattgctcagacgaagcttcctgttactaagatgtcttaagtggaatagagtcattccaagaactacaaactaaagctactgtagaaacaaagggttttcttttcgaatgtttcttggtagtttctcataatgtgagacggttcccagtatcatgtgatcttctcctccagactcctcttctttatgttccaagactgtgcaatactttagacgccctcgcacctctctcttcccatgtggaatgggacgcccacctacagtctaatgagtaaactttcagttttttgtttgtttgtttttttttagattcaagcaagtatgaatctagttgttggataccttttttcatgatgtaataaagtattttctttaaaagttattgcaa

SEQ ID NO: 35 - DNA, 918 bp, Mus musculus
  misc_feature (1)..(918): TGIF1 Mouse CDS >NM_001164075.1311-1228 Mus musculus TGFB-induced factor homeobox 1 (Tgif1), transcript variant 1, mRNA
atggagatggtgctctcgccacgggcaggcaggcatgaccctgcttctgggttccgccagcgcgtagagggacgggtgatctcggcccccaggcccagggttgtggagctggaaggtcttgttgcagcatctggcagtgactctgaggatgaagacagcatggacagtcccctggacctttcctcatcagcagcctctggcaagagaaggaggagaggcaatctgcccaaggagtcagtccagattctgcgagactggctgtatgaacacagatacaacgcctatccctcagagcaagagaaagcactgctgtcccagcagacacacctgtccacactacaggtctgtaactggttcatcaacgcccgccgcaggctccttcctgacatgctgagaaaggatggcaaagatccaaatcagttcacgatttcccgccgtggggccaagatttcagaagctagctctattgaagctgcaatgggtatcaaaaacttcatgccaactctagaagagagcccatttcattcctgcgtagttggacccaacccaaccctagggagaccagtgtctcccaaacctccctccccaggatccattttggctcgcccgtcagtgatctgccataccactgtgactgcattgaaggatgggcctttctctctctgtcagccgattggtgtgggacagagtacagatgtaccgcaaatagcacccagcaactttacagacacctctctcgtgtacccagaggacacttgcaaatctggacccagtccaaaccctcagagtggtcttttcaacactcctccccctactccaccagacctcaaccaggattttagtggattccagcttctagtggatgttgcactcaaacgagcggcagagatggagcttcaggccaaactcacagcttaa

SEQ ID NO: 36 - protein, 305 aa, Mus musculus
  MISC_FEATURE (1)..(305): TGIF1 Mouse Protein >NP_001157547.1 homeobox protein TGIF1 isoform a [Mus musculus]
MEMVLSPRAGRHDPASGFRQRVEGRVISAPRPRVVELEGLVAASGSDSEDEDSMDSPLDLSSSAASGKRRRRGNLPKESVQILRDWLYEHRYNAYPSEQEKALLSQQTHLSTLQVCNWFINARRRLLPDMLRKDGKDPNQFTISRRGAKISEASSIEAAMGIKNFMPTLEESPFHSCVVGPNPTLGRPVSPKPPSPGSILARPSVICHTTVTALKDGPFSLCQPIGVGQSTDVPQIAPSNFTDTSLVYPEDTCKSGPSPNPQSGLFNTPPPTPPDLNQDFSGFQLLVDVALKRAAEMELQAKLTA

SEQ ID NO: 37 - DNA, 2258 bp, Homo sapiens
  misc_feature (1)..(2258): RELB Human Full sequence >NM_006509.4 RELB proto-oncogene, NF-kB subunit, mRNA [Homo sapiens]
gcagccccgggcgccgcgcgtcctgcccggcctgcggccccagcccttgcgccgctcgtccgacccgcgatcgtccaccagaccgtgcctcccggccgcccggccggcccgcgtgcatgcttcggtctgggccagcctctgggccgtccgtccccactggccgggccatgccgagtcgccgcgtcgccagaccgccggctgcgccggagctgggggccttagggtcccccgacctctcctcactctcgctcgccgtttccaggagcacagatgaattggagatcatcgacgagtacatcaaggagaacggcttcggcctggacgggggacagccgggcccgggcgaggggctgccacgcctggtgtctcgcggggctgcgtccctgagcacggtcaccctgggccctgtggcgcccccagccacgccgccgccttggggctgccccctgggccgactagtgtccccagcgccgggcccgggcccgcagccgcacctggtcatcacggagcagcccaagcagcgcggcatgcgcttccgctacgagtgcgagggccgctcggccggcagcatccttggggagagcagcaccgaggccagcaagacgctgcccgccatcgagctccgggattgtggagggctgcgggaggtggaggtgactgcctgcctggtgtggaaggactggcctcaccgagtccacccccacagcctcgtggggaaagactgcaccgacggcatctgcagggtgcggctccggcctcacgtcagcccccggcacagttttaacaacctgggcatccagtgtgtgaggaagaaggagattgaggctgccattgagcggaagattcaactgggcattgacccctacaacgctgggtccctgaagaaccatcaggaagtagacatgaatgtggtgaggatctgcttccaggcctcatatcgggaccagcagggacagatgcgccggatggatcctgtgctttccgagcccgtctatgacaagaaatccacaaacacatcagagctgcggatttgccgaattaacaaggaaagcgggccgtgcaccggtggcgaggagctctacttgctctgcgacaaggtgcagaaagaggacatatcagtggtgttcagcagggcctcctgggaaggtcgggctgacttctcccaggccgacgtgcaccgccagattgccattgtgttcaagacgccgccctacgaggacctggagattgtcgagcccgtgacagtcaacgtcttcctgcagcggctcaccgatggggtctgcagcgagccattgcctttcacgtacctgcctcgcgaccatgacagctacggcgtggacaagaagcggaaacgggggatgcccgacgtccttggggagctgaacagctctgacccccatggcatcgagagcaaacggcggaagaaaaagccggccatcctggaccacttcctgcccaaccacggctcaggcccgttcctcccgccgtcagccctgctgccagaccctgacttcttctctggcaccgtgtccctgcccggcctggagccccctggcgggcctgacctcctggacgatggctttgcctacgaccctacggcccccacactcttcaccatgctggacctgctgcccccggcaccgccacacgctagcgctgttgtgtgcagcggaggtgccggggccgtggttggggagacccccggccctgaaccactgacactggactcgtaccaggccccgggccccggggatggaggcaccgccagccttgtgggcagcaacatgttccccaatcattaccgcgaggcggcctttgggggcggcctcctatccccggggcctgaagccacgtagccccgcgatgccagaggaggggcactgggtggggagggaggtggaggagccgtgcaatcccaaccaggatgtctagcacccccatccccttggcccttcctcatgcttctgaagtggacatattcagccttggcgagaagctccgttgcacgggtttccccttgagcccattttacagatgaggaaactgagtccggagaggaaaagggacatggctcccgtgcactagcttgttacagctgcctctgtccccacatgtgggggcaccttctccagtaggattcggaaaagattgtacatatgggaggagggggcagattcctggccctccctccccagacttgaaggtggggggtaggttggttgttcagagtcttcccaataaagatgagtttttgagcc

SEQ ID NO: 38 - DNA, 1740 bp, Homo sapiens
  misc_feature (1)..(1740): RELB Human CDS > NM_006509.4117-1856 RELB proto-oncogene, NF-kB subunit (RELB), mRNA, CDS [Homo sapiens]
atgcttcggtctgggccagcctctgggccgtccgtccccactggccgggccatgccgagtcgccgcgtcgccagaccgccggctgcgccggagctgggggccttagggtcccccgacctctcctcactctcgctcgccgtttccaggagcacagatgaattggagatcatcgacgagtacatcaaggagaacggcttcggcctggacgggggacagccgggcccgggcgaggggctgccacgcctggtgtctcgcggggctgcgtccctgagcacggtcaccctgggccctgtggcgcccccagccacgccgccgccttggggctgccccctgggccgactagtgtccccagcgccgggcccgggcccgcagccgcacctggtcatcacggagcagcccaagcagcgcggcatgcgcttccgctacgagtgcgagggccgctcggccggcagcatccttggggagagcagcaccgaggccagcaagacgctgcccgccatcgagctccgggattgtggagggctgcgggaggtggaggtgactgcctgcctggtgtggaaggactggcctcaccgagtccacccccacagcctcgtggggaaagactgcaccgacggcatctgcagggtgcggctccggcctcacgtcagcccccggcacagttttaacaacctgggcatccagtgtgtgaggaagaaggagattgaggctgccattgagcggaagattcaactgggcattgacccctacaacgctgggtccctgaagaaccatcaggaagtagacatgaatgtggtgaggatctgcttccaggcctcatatcgggaccagcagggacagatgcgccggatggatcctgtgctttccgagcccgtctatgacaagaaatccacaaacacatcagagctgcggatttgccgaattaacaaggaaagcgggccgtgcaccggtggcgaggagctctacttgctctgcgacaaggtgcagaaagaggacatatcagtggtgttcagcagggcctcctgggaaggtcgggctgacttctcccaggccgacgtgcaccgccagattgccattgtgttcaagacgccgccctacgaggacctggagattgtcgagcccgtgacagtcaacgtcttcctgcagcggctcaccgatggggtctgcagcgagccattgcctttcacgtacctgcctcgcgaccatgacagctacggcgtggacaagaagcggaaacgggggatgcccgacgtccttggggagctgaacagctctgacccccatggcatcgagagcaaacggcggaagaaaaagccggccatcctggaccacttcctgcccaaccacggctcaggcccgttcctcccgccgtcagccctgctgccagaccctgacttcttctctggcaccgtgtccctgcccggcctggagccccctggcgggcctgacctcctggacgatggctttgcctacgaccctacggcccccacactcttcaccatgctggacctgctgcccccggcaccgccacacgctagcgctgttgtgtgcagcggaggtgccggggccgtggttggggagacccccggccctgaaccactgacactggactcgtaccaggccccgggccccggggatggaggcaccgccagccttgtgggcagcaacatgttccccaatcattaccgcgaggcggcctttgggggcggcctcctatccccggggcctgaagccacgtag

SEQ ID NO: 39 - protein, 579 aa, Homo sapiens
  MISC_FEATURE (1)..(579): RELB Human Protein >NP_006500.2 transcription factor RelB [Homo sapiens]
MLRSGPASGPSVPTGRAMPSRRVARPPAAPELGALGSPDLSSLSLAVSRSTDELEIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIELRDCGGLREVEVTACLVWKDWPHRVHPHSLVGKDCTDGICRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPNHGSGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 40 - DNA, 2194 bp, Mus musculus
  misc_feature (1)..(2194): RELB Mouse Full sequence >NM_001290457.1 Mus musculus avian reticuloendotheliosis viral (v-rel) oncogene related B (Relb), transcript variant 2, mRNA [mus musculus]
gcccgccgcccgctcggcccggcgccccccgcagccccgggcgccgtgcgtccagcccggccttcggccctagccttgcaccgcttgcccggccggtgatcgtcccagccgaccgggctgtccgactgcccggcttgccctcgtgtgcgctcgccctgagctggccttcgggctgtcggtccctacgggccgggccatgccgagtcgccgcgctgccagagagtccgcgcccgagctaggggccttgggttccagtgacctctcttccctgtcactaacggtctccaggaccacagaaatcatcgacgaatacattaaggagaacggctttggcctggacgggacacagctgagtgagatgccgcgcctggtgccccgcgggcccgcctcactgagcagcgtcacgctgggccctgctgcaccaccgcctccggccacgccgtcctggagctgcacactgggcaggctggtgtcacccggcccgtgcccacggccgtacctggtcatcacagagcagccaaagcagcgtggcatgcgcttccgctacgagtgcgagggccgctcggccggcagcatcctcggggagagcagcaccgaagccagcaagaccctgcccgccatcgagcttcgagactgtggcgggctgcgggaggtggaggtgacggcgtgcctggtgtggaaggactggccacaccgggtacacccacatagcctcgtggggaaagactgcacggacggcgtctgcagggtgcggctgcggcctcacgtcagcccccggcacagctttaacaacctgggcatccagtgtgttaggaagaaggaaattgaagctgccattgagcggaagatccagctgggaattgacccctacaatgctggctccctgaagaaccatcaggaggtcgacatgaatgtcgtcaggatctgcttccaggcctcctatcgggaccagcagggacatctgcaccgcatggaccccatcctctctgagcctgtctacgacaagaagtccaccaacacatcggagctgcggatttgccgaatcaacaaggagagcgggccgtgcacaggtggtgaggagctgtacttgctctgtgacaaggtgcaaaaagaggacatatccgtggtgttcagcacagcttcctgggaaggccgtgccgacttctctcaagctgatgtgcaccggcagatcgccattgtgttcaaaacgccaccctacgaggacctggagatctcagagcccgtgactgtcaatgtgttcttgcagcggctcacggatggggtgtgcagcgagccgctgcccttcacgtacctgcctcgggatcatgacagctacggtgtggacaagaagcgaaagcggggactgcctgatgtccttggagagttgagcagctctgatccacatggaatcgagagcaaacgaaggaaaaagaaaccagtgttcttggaccacttcctgcctggccacagctcaggcctgttcctcccaccatcggctctgcagccggcagactctgatttcttccctgcttccatatcccttcctgggctggagcctcctggtggacccgatctcctggacgatggctttgcctatgatccttctgcccccacgctcttcactatgttggacctgctgcccccagcaccaccacttgccagtgctgtggtgggtagcgggggtgcaggggccaccgttgtggagtcttctggcccagagcccctatcactggactcttttgcagcgccgggccccggggatgttggtactgctagccttgtgggcagcaacatgtttcccaaccagtaccgagaggcagctttcgggggtggcctcctatctccagggcctgaagccacgtagcctctgaggtaacagaggaggcactgggtgaggtatgtggtatagcactccattccgaagccaaccttgatcagtcttccagcttcctcatcctgaatcggacatctgcagcgctggtgggaagatggggagcactccggttctctttgagcccattttacagaatgctgagtccgaagaggaaaaggggctcctgcagatggaccccttctcaggacagattctcagagattgtacataggggaggagggagcaggtccccagccttctcccctaatcctgaagaaggcagtggattgttcagttttcccaataaaaattagttttttg

SEQ ID NO: 41 - DNA, 1668 bp, Mus musculus
  misc_feature (1)..(1668): RELB mouse CDS >NM_001290457.1197-1864 Mus musculus avian reticuloendotheliosis viral (v-rel) oncogene related B (Relb), transcript variant 2, mRNA [mus musculus]
atgccgagtcgccgcgctgccagagagtccgcgcccgagctaggggccttgggttccagtgacctctcttccctgtcactaacggtctccaggaccacagaaatcatcgacgaatacattaaggagaacggctttggcctggacgggacacagctgagtgagatgccgcgcctggtgccccgcgggcccgcctcactgagcagcgtcacgctgggccctgctgcaccaccgcctccggccacgccgtcctggagctgcacactgggcaggctggtgtcacccggcccgtgcccacggccgtacctggtcatcacagagcagccaaagcagcgtggcatgcgcttccgctacgagtgcgagggccgctcggccggcagcatcctcggggagagcagcaccgaagccagcaagaccctgcccgccatcgagcttcgagactgtggcgggctgcgggaggtggaggtgacggcgtgcctggtgtggaaggactggccacaccgggtacacccacatagcctcgtggggaaagactgcacggacggcgtctgcagggtgcggctgcggcctcacgtcagcccccggcacagctttaacaacctgggcatccagtgtgttaggaagaaggaaattgaagctgccattgagcggaagatccagctgggaattgacccctacaatgctggctccctgaagaaccatcaggaggtcgacatgaatgtcgtcaggatctgcttccaggcctcctatcgggaccagcagggacatctgcaccgcatggaccccatcctctctgagcctgtctacgacaagaagtccaccaacacatcggagctgcggatttgccgaatcaacaaggagagcgggccgtgcacaggtggtgaggagctgtacttgctctgtgacaaggtgcaaaaagaggacatatccgtggtgttcagcacagcttcctgggaaggccgtgccgacttctctcaagctgatgtgcaccggcagatcgccattgtgttcaaaacgccaccctacgaggacctggagatctcagagcccgtgactgtcaatgtgttcttgcagcggctcacggatggggtgtgcagcgagccgctgcccttcacgtacctgcctcgggatcatgacagctacggtgtggacaagaagcgaaagcggggactgcctgatgtccttggagagttgagcagctctgatccacatggaatcgagagcaaacgaaggaaaaagaaaccagtgttcttggaccacttcctgcctggccacagctcaggcctgttcctcccaccatcggctctgcagccggcagactctgatttcttccctgcttccatatcccttcctgggctggagcctcctggtggacccgatctcctggacgatggctttgcctatgatccttctgcccccacgctcttcactatgttggacctgctgcccccagcaccaccacttgccagtgctgtggtgggtagcgggggtgcaggggccaccgttgtggagtcttctggcccagagcccctatcactggactcttttgcagcgccgggccccggggatgttggtactgctagccttgtgggcagcaacatgtttcccaaccagtaccgagaggcagctttcgggggtggcctcctatctccagggcctgaagccacgtag

SEQ ID NO: 42 - protein, 555 aa, Mus musculus
  MISC_FEATURE (1)..(555): RELB Mouse protein >NP_001277386.1 transcription factor RelB isoform 2 [Mus musculus]
MPSRRAARESAPELGALGSSDLSSLSLTVSRTTEIIDEYIKENGFGLDGTQLSEMPRLVPRGPASLSSVTLGPAAPPPPATPSWSCTLGRLVSPGPCPRPYLVITEQPKQRGMRFRYECEGRSAGSILGESSTEASKTLPAIELRDCGGLREVEVTACLVWKDWPHRVHPHSLVGKDCTDGVCRVRLRPHVSPRHSFNNLGIQCVRKKEIEAAIERKIQLGIDPYNAGSLKNHQEVDMNVVRICFQASYRDQQGHLHRMDPILSEPVYDKKSTNTSELRICRINKESGPCTGGEELYLLCDKVQKEDISVVFSTASWEGRADFSQADVHRQIAIVFKTPPYEDLEISEPVTVNVFLQRLTDGVCSEPLPFTYLPRDHDSYGVDKKRKRGLPDVLGELSSSDPHGIESKRRKKKPVFLDHFLPGHSSGLFLPPSALQPADSDFFPASISLPGLEPPGGPDLLDDGFAYDPSAPTLFTMLDLLPPAPPLASAVVGSGGAGATVVESSGPEPLSLDSFAAPGPGDVGTASLVGSNMFPNQYREAAFGGGLLSPGPEAT

SEQ ID NO: 43 - DNA, 6212 bp, Homo sapiens
  misc_feature (1)..(6212): RBPJ Human Full sequence >NM_001363577.2 recombination signal binding protein for immunoglobulin kappa J region (RBPJ), transcript variant 5, mRNA [Homo sapiens]
gccgtggctcaggctccagcgcgtgagggcgggcggcggcggcggcggcgaggccggcgggtgcgggcgtgagtgcggcagggcgcgcggggccctcgggcgccgtgcctcccgcgcgcctccagcgggctctgcctgggggccccgtgcatctttgttcctcgcggccgttgtcgccgcttcgccccgctagtgcccccggccccgggcgctttgtctttcaccgctacccgctgcgactctctacccccggccctttgtttcgccgccttgggcgctttgtccgacccgtggcgtcccgcatcccctctccatctcctcccctccgctgtccttcctcccgtgcccctttgttgtgccacactcttgtcgttagcccagtgacctgccacagcccgggcaccagatttctgccttaattgttcttccattgtctttctcctgtgggtcccctctcacctttctgtatggtcctggatcaccccccgaggctttgtctcccccatccacgggcttattctctcggcacccccttcctctcccgtcatcggttgatttatccacaacccacggtgtacgtctgctgaactgtctcagccccggggactccatctgctgcttctaagtgcacacaagactctacggaaatttggtgagcggcctccacctaaacgacttactagggaagctatgcgaaattatttaaaagagcgaggggatcaaacagtacttattcttcatgcaaaagttgcacagaagtcatatggaaatgaaaaaaggtttttttgcccacctccttgtgtatatcttatgggcagtggatggaagaaaaaaaaagaacaaatggaacgcgatggttgttctgaacaagagtctcaaccgtgtgcatttattgggataggaaatagtgaccaagaaatgcagcagctaaacttggaaggaaagaactattgcacagccaaaacattgtatatatctgactcagacaagcgaaagcacttcatgttgtctgtaaagatgttctatggcaacagtgatgacattggtgtgttcctcagcaagcggataaaagtcatctccaaaccttccaaaaagaagcagtcattgaaaaatgctgacttatgcattgcctcaggaacaaaggtggctctgtttaatcgactacgatcccagacagttagtaccagatacttgcatgtagaaggaggtaattttcatgccagttcacagcagtggggagccttttttattcatctcttggatgatgatgaatcagaaggagaagaattcacagtccgagatggctacatccattatggacaaacagtcaaacttgtgtgctcagttactggcatggcactcccaagattgataattaggaaagttgataagcagaccgcattattggatgcagatgatcctgtgtcacaactccataaatgtgcattttaccttaaggatacagaaagaatgtatttgtgcctttctcaagaaagaataattcaatttcaggccactccatgtccaaaagaaccaaataaagagatgataaatgatggcgcttcctggacaatcattagcacagataaggcagagtatacattttatgagggaatgggccctgtccttgccccagtcactcctgtgcctgtggtagagagccttcagttgaatggcggtggggacgtagcaatgcttgaacttacaggacagaatttcactccaaatttacgagtgtggtttggggatgtagaagctgaaactatgtacaggtgtggagagagtatgctctgtgtcgtcccagacatttctgcattccgagaaggttggagatgggtccggcaaccagtccaggttccagtaactttggtccgaaatgatggaatcatttattccaccagccttacctttacctacacaccagaaccagggccgcggccacattgcagtgcagcaggagcaatccttcgagccaattcaagccaggtgccccctaacgaatcaaacacaaacagcgagggaagttacacaaacgccagcacaaattcaaccagtgtcacatcatctacagccacagtggtatcctaactaccgtctttttgctaggacttaaactgacttgagtgtggcaaaaagttaacaaaaaaggagaaaaaatgaacaatcgtttgtggtttcttgggaaaacttttcataccaggtgatactattcaaaaaccccgttgtctccctgcaagtgctgatttgaaatgcagaagccacagtaaaaaaaaaaaaaaaaaaaaaaaaaaagaaaaaaaaatcaaaatgtataaatattggaaatcaagtttttcagctgttttgttggttggttggttggtttttgtttggttttgtttaaatgggcaagaagtaaataatgtggctggaatacaagttgaacaaactagaagacacaaatctaacatagtttttatggaccaaggaacttgtatattgtataagctttagtaaaaggtacattttcaccatacctttttttatatcacggtattatagtacaccttgttaccaaataggttgttctcttccccacccacctttgagcttttgctctaaaatacattcaggttccaagcctgaccatccttgtttaatctatcatactcttccaggttttttttttttggtctaaggctggaacttttttcttttttttcagctgaagtcttatgacttttcatgagtcaaaattgtttggatttcagcaagtcaaatcttgcaaaggcctgcatattttttttaagattatatgaagtctgtgcaaaagctttaaaaaaatgcctctgccttgcctgcaatacatgcaatgtatgttaacttagtctctcttctcagacactgttggtagttatttctgtgttttcctttttttaaaaaaaaatatggacttattgtggttatctgagaggttctaacattcacatgcaatttggtgtggccatttagctattaatgagttaatggcgcagaacttgttgatatttgaagtgttctctccccttttcccatgacgtaaatacataggtgtgttccaggatttgttcaggtttttcccccctcctaatcttgtacataacttgtattatgtgtaagttaaacattttattttgaacttggaatgttcccagtgatttcattcagcagggtattttctgccttgttggcaagtgacaaaaaatatgggaagtatttgctaccagttggtagatggtgcccttaatggtagaatgaggaaaatgtccgcaaaagcatgttttattatctttacttttttggggggttggagggggtagcctagccagaacatcattgtaatcttaaaacataagatgcttttattagatgatcaactaaaatagctggaagacagtactttagaaacagatagttgtaagattataaaatgcaaatgtaacttatgttttcatttttttctctgccttttttgtttgtttgttttctcttttccagtactgagcatctccacaaatgtctcctaactcagaaaatgtttcttttcttttcagttgagatttggttgcattcagggttgtaggttggccttgcttgctaaccccgccggttttaccgtgctttcattcctgaactttgtttatgcctttgtttggtttcttcgaaattgcagcagactcattgggctacatttagtacaggaaccacgtgtgtaatgttatacaacacagtctagtaatacaatcatccctcttagagtaaaaactacctctagattgtggtaagcttttactgtcccataaaacaggagccacagtaccttatgaatgcaaaactgtaacttcctacagtgtttccctacagaacattgtctttctggtgtcctgggctgttttgaaaaagtttccattaatagactttttagaaattattattagtagcattttttttccagctttgctgtcttcatcactcactctatgctcagactatgccactgtaaatattcttcctaacatctttaaatcgccttttcctcagttttcaaggggaaggtcatttgtaaagcacgttaggtggttaaatcagttattgcggttttctcttactgcaagcctttttaatcacccccaggctgcattttattctatatcgccttttttcttcaaatctgctccaatcactcacttctctcttataagctaatcctgcctcacaccttaaatctgtttcagtgatcaagggcagaactcattgtggccttatctttctttgttgtaattgttcactgtctctttcttacagaccacttatttctgagtagtagttattcctctctatggagtcatggcaggaatcattacacagtgcttttgttcagagcatggacatgttcctagtgctgctttgctttaacggccacaagtttcctccacttcctaggtttggtatttagttaaggaatcatattaaattaaccaataacaaaagagatacttttgaagaacaaactattccttacccatttttgtagctcaaaaataatttttcaagttcatgaccttattaaaatgaacttgtgtttttttaacaaacatgtatgttttattttgatagtttctttccgtaagataattgaaatattatactgtaaacccttttcttttctttttttgaaaagtccaagaatgtacttatacaggcatttttccccacctatttttggccattctcataccacagactaaagagtgaaatgatttgtccattgtagcttattgtttatcagtagttcttttgtcagctgcttacattttttctttcatggttttgtgaatcattttcagtatgtaatttataggaaccttgtcctctggttatagtagactgtgtgccctcctccagtgatggcattattagacatgctggtcatttaccctcagaaagactctcttattagaatggtgagtgcttcagttatagtatgtttgaatttttaaaaaattctgttttagaaatgtatcttatgctctcatgactatgcagtttctaaacatacacatagaagctgagtctctgatccaatatgtttttatttgttccatttaatttatcacatagattgggaaggcaagctaaaagccttaaaaatgccctttatattttgagtgatttcagcgttgaacactagtatactatctaaatttgctgctcactttctttaaactgtggcaattaaaggcatgtttatacatgacttaatcgtgaaatgtttgtcactcttactgcacagacttatctgcaatcataactggttagtttttttgttttgttttgttttattgtttttaatgaaactggtaccatctgtgctttcacaaaaaacttccaatgccatttttgagaactaacctaactagtcatgctaaccagaaaatccactggggaggaggttccttttgaaacaaaatgctgttcagttagtaaccaagttactttgattgcaaaagcagctgtgtttctgataagtactgaacaaatgtgtgtaattttctgtgccagacttatgactttgttttcaagcactgtaatgtgggatggatggttagaaacaataatatattagggtttctgtttaaccctttcaggactgaactgtatctccttttgttaattttcccctgtgttgtgataaatgtttgccagcattcagtactgtgttggtccagatgtaggtttatatgctcatttttagcttatttcttgtaccttgcagcatgctctacgcattcagtccttaaggggtttattttacaaactgtgcgcctgtaaggtttattagcaataagatagaaaattgagcaagtttataccataattttgtagaaaaaaagaatctgctcagttccatatttcatccgtgaaaaacttgcaatacgagcagtttcaaggaataaataaaaaggaaatgtaaaccattgtaaaagtcttctgtcgaatgtgcctgatgcatgtattatcgtcttttatttcagaatacttcataaagataaaattaaattctatattatagttggtgtatttacaatcttaccatgtacatcacatcaaagtgatagctctactaatttaatttccttgtcaatgtttttaactatatagtgctttaaagagattttttttccctgtgtaaa

SEQ ID NO: 44 - DNA, 1398 bp, Homo sapiens
  misc_feature (1)..(1398): RBPJ Human CDS >NM_001363577.2691-2088 recombination signal binding protein for immunoglobulin kappa J region (RBPJ), transcript variant 5, mRNA [Homo sapiens]
atgcgaaattatttaaaagagcgaggggatcaaacagtacttattcttcatgcaaaagttgcacagaagtcatatggaaatgaaaaaaggtttttttgcccacctccttgtgtatatcttatgggcagtggatggaagaaaaaaaaagaacaaatggaacgcgatggttgttctgaacaagagtctcaaccgtgtgcatttattgggataggaaatagtgaccaagaaatgcagcagctaaacttggaaggaaagaactattgcacagccaaaacattgtatatatctgactcagacaagcgaaagcacttcatgttgtctgtaaagatgttctatggcaacagtgatgacattggtgtgttcctcagcaagcggataaaagtcatctccaaaccttccaaaaagaagcagtcattgaaaaatgctgacttatgcattgcctcaggaacaaaggtggctctgtttaatcgactacgatcccagacagttagtaccagatacttgcatgtagaaggaggtaattttcatgccagttcacagcagtggggagccttttttattcatctcttggatgatgatgaatcagaaggagaagaattcacagtccgagatggctacatccattatggacaaacagtcaaacttgtgtgctcagttactggcatggcactcccaagattgataattaggaaagttgataagcagaccgcattattggatgcagatgatcctgtgtcacaactccataaatgtgcattttaccttaaggatacagaaagaatgtatttgtgcctttctcaagaaagaataattcaatttcaggccactccatgtccaaaagaaccaaataaagagatgataaatgatggcgcttcctggacaatcattagcacagataaggcagagtatacattttatgagggaatgggccctgtccttgccccagtcactcctgtgcctgtggtagagagccttcagttgaatggcggtggggacgtagcaatgcttgaacttacaggacagaatttcactccaaatttacgagtgtggtttggggatgtagaagctgaaactatgtacaggtgtggagagagtatgctctgtgtcgtcccagacatttctgcattccgagaaggttggagatgggtccggcaaccagtccaggttccagtaactttggtccgaaatgatggaatcatttattccaccagccttacctttacctacacaccagaaccagggccgcggccacattgcagtgcagcaggagcaatccttcgagccaattcaagccaggtgccccctaacgaatcaaacacaaacagcgagggaagttacacaaacgccagcacaaattcaaccagtgtcacatcatctacagccacagtggtatcctaa

SEQ ID NO: 45 - protein, 465 aa, Homo sapiens
  MISC_FEATURE (1)..(465): RBPJ Human Protein NP_001350506.1 recombining binding protein suppressor of hairless isoform 5 [Homo sapiens]
MRNYLKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFFIHLLDDDESEGEEFTVRDGYIHYGQTVKLVCSVTGMALPRLIIRKVDKQTALLDADDPVSQLHKCAFYLKDTERMYLCLSQERIIQFQATPCPKEPNKEMINDGASWTIISTDKAEYTFYEGMGPVLAPVTPVPVVESLQLNGGGDVAMLELTGQNFTPNLRVWFGDVEAETMYRCGESMLCVVPDISAFREGWRWVRQPVQVPVTLVRNDGIIYSTSLTFTYTPEPGPRPHCSAAGAILRANSSQVPPNESNTNSEGSYTNASTNSTSVTSSTATVVS

SEQ ID NO: 46 - DNA, 5590 bp, Mus musculus
  misc_feature (1)..(5590): Mouse RBPJ Full sequence >NM_001277116.1 recombination signal binding protein for immunoglobulin kappa J region (Rbpj), transcript variant 4, mRNA [Mus musculus]
agtaatgccctccggttttcctcagtctccacgtacgtccccgagggcgcgtcccaaaacccggataaccggagcgctccccatggactactcggagggcttgtccgcggaggagcggcctgcgcatgctccatcggcgggctcctggccttgtgcccttgaggcaagcattctacagattacatacttctacactccagaagagggcgtcagatctcgttacggatggttgtgagccaccatgtggttgctgggatttgaactcctgaccttcagaagagcagtcgggtgctcttacccattgagccatctcaccagcccgaagtttggtgagcggcctccacccaaacgactcactagggaagctatgcgaaattatttaaaagaacgaggggatcaaacagtgctcattcttcatgcaaaagttgcacagaagtcttacggaaatgaaaaacgatttttttgccctcctccttgtgtgtatcttatgggcagtggttggaagaaaaaaaaagagcaaatggaacgagatggatgttctgaacaagagtctcaaccctgtgcgtttattggaataggaaacagtgaccaagaaatgcagcagctgaacttggaagggaagaactactgtacagccaaaacattgtacatatctgattcagacaagagaaaacatttcatgttgtctgtaaagatgttctatggcaacagcgatgacattggtgtgttcctcagcaagcggataaaggtcatctccaaaccctccaaaaagaagcagtcactgaagaatgctgacttgtgcattgcttcaggaacgaaggtggcactgttcaatcgccttcggtcccagacagttagtaccaggtacctgcatgtagaaggagggaatttccacgccagttcacaacagtggggagcattttacatccatctcttggacgacgacgagtcggaaggagaggagttcacagttagagatggctacatccattacgggcagactgtcaagcttgtgtgctcagtgactggcatggcactcccaagattgataattaggaaagttgataagcagacggcattactggatgcagacgaccctgtatcacaactccacaaatgtgcattttaccttaaggatacagaaagaatgtacttgtgcctttctcaagaaagaataatccaatttcaggccactccatgtccaaaagaacaaaataaggaaatgataaacgatggagcttcctggacaatcattagcacagacaaggccgagtacacgttctatgagggaatgggccccgtccttgccccagtcacccctgtgcctgtcgtagaaagtcttcagttgaatggcggcggggacgtagcaatgcttgaacttacaggacaaaactttactccaaatttaagagtgtggtttggggatgtagaagccgaaacaatgtacagatgtggagagagcatgctctgtgtggtcccagacatttctgcattccgggaaggttggagatgggtccgccagccagtccaggttccagtgactttggtccgaaatgacggggtcatttactccaccagccttaccttcacctacacaccagagccagggccgaggccacactgcagcgctgcaggagcgattctcagagccaactccagccaagtgccctccaatgagtcaaacacaaacagcgaggggaattacacaaatgccagcacaaattctaccagtgtcacatcgtccacagcaaccgtggtgtcctaactaccgtctttttgctgggactgaaactgacttgaatgcagcaaaaagttgacagagaaaggcaaggataaagtgaacaatcttttgtggtttcttggggaaacgtttcgtaccaggtgatctattcaaaactgccagtgcaggtgtgacatgcagaggccacagtacaatgaacaacatcagaatgcacaggctgatggaaatccaggttttcagctgtccctctcacacgcacccctttttggttgttttggtttggttttgatttaatggccaggaaacagatatgtggctggagcacaggctaagctagaagacacaaacctaacatagacttatggaccaagggactcgcataagctttagtgaaaggtacattttcaccatgccttttttatatcacagtgtgtactacaccttgttaccaaacaggttgcgctccccaccctttgagggttggctctaaagtacattcattcaggttcgagcctcaccatgtttgttcagtctgaacactgaacacttccaggttcttgtggtctaaggctggaacttttttaagctgaagtcttatgactttttcataagttggaattattttgatttcagcaagtcaaattttgtaaaggcctgcatattttttttaagattatatgaagtctgcaaaagctttaaaaatgcctctgccttgcctgcaatacatgcaatgtgtgttaactgagtctgttctcagaatctgttggtagttatttctgtgttttccctttttttaaagaaatgtatttatagtggtaatctaagagatgctaacagctccgtgtggccatttagccctttctgagttcatggcagtgagcactgccgttcgaaggctgtgcgcctctgcctgttcatcagttcatctgtgttccaggatttgttcaggtttccccttcccccaaatcttgtacataacttgtattatgtgtaagttaaacattttatctttacttggaatgttcccagtgattacatttcacagggtgtttcccaccttgttggcaaatgacaaaaaatatcatgcgaattatttgctaccaagatggtgcccgtagggtaaaatgaggaacatctctgcaggatgatttattgtatgttttcctttttttttagcctagccagaacatcattataattttaaagaccataagatggcttttattagatgataactaaataaactgaactaagtagccagaagacagtaccttagagactgatagttgtaagtttataaaaatacaaatgtaacttatatttccattttcctctttgcccctttgtttattttccccagtagaaatgttctgtccagatttttttccccctttttagttaagccttgcttgcattcaggcttgtgtgtcagccctgtgtgccaatatgccagattcattttgtttccatccctgcactttggttattccttgttcagtttcttgggaattgaagcagactcattgggctacatttagtacaagaaccacatgtgtgatgttaaaggacacagtctagtgatgccatcatccttgagtaaaaactacctctagattgtggtaagcttttactgtcccttaaaacaggagccactagtaccttatgaatgcagactgtaacttctatggcattttctggtgtctttctggctctctgggcttctgaaagttttcacgtagacccttagactcctctgtcaggaccattgctttccagcgttgctgctttcactcgctgtctcgaagactgtaccactgtaagtgccactgcgaacatctttaaacggctcctccttggcttgtcaaggggaaggccacctgtaagatgtggtggtgacagccttcctgtccaccccaggctgctccttaccagctgttctagccgattctcttcagatcagctacagcccacctcctccccgccccgcctcacccctccaggctgtctcagtggtccagggcagagctcactgtggccttactgatctatctctgcagtaactgttctccctcagttgcgatggaagcaggcccctgcgatctgttcagagcctgagcagtttgcagttccgttgtgctttctgaggtcctagattcaggatgtagttaagtcgataactgagaacagatgagatgcttttaaagaacaaactcttccttacccagctttgtagcttaaagtaagttttcaagttcatggctttattaaaatgaactttgactttttaaaatgcttatatctcatttcggttcttttgtttattttagtaagaattgaaacagactataagtagttttcctgtttgtttgtttgtttgtttgttttttacaaagttcaagaatgtaacaataaaggcattttgctatggttctatgtcagactcctaccacagaatagtgtgaacagatatgtctgtctgctttaccatgttattgatagttcttttgtcagctgcttttattaaaagtcctcttttgtgaatttgtaaatcaatttcaatatatgatgtacaggagtcttgtcccccgttactgtggaatgttcccccctgttgatgctggtccctttatcctcttatcttcttagaaccgggaattcttcagttgtggtgtgtggctgtatttttctattctgtttacagtgcgtctacacagcttcttaaaacatgtcgaaatgcagaatccaagatccaccatgaatttgtcaagagattggaaggcaggccaaaacctgaaaatgtggggtgccctttgtacaattgcagagtcaagcactagtgggagtttgaactggctactgaccgctcttaaactgtggcagtgtccttgggtacagttaactagcagaacctgctgtcactcttcctgccaccatcttaaccttgacagttttgtttcttttctgtgtggtgctggggtcaaatccagagccttaggcttgctaaggctttttgtttgtttgatttttccttgagacaagtgtctcacccatggagcccctcagcattcttgaaagtctctgccttagcctccaggtcctgtggtaccaggtgtggcaggatgcttttgtcattagagaaactggtctgtgtccaaccacagaaaaattccaatgcagtttttaataattaatccactagttatttaatcaaaatctttttgtagggagagattcctgtttgaaagaaaatgctatttgattattaaccaaggtaacagacactgctttccaaagcagctgtgtttctggtgagtactgaacaaatgtgtggttttctgtgccacctttgattcatttttcaagcactgttactcgggatggatggttagaaacatgatattttagtgtttctacttaaccctttcaggaccgagctgtattgcctttctattaatttccccctgtgttgtgataaatgtttgctcgtgttcagtcctgtgtcagtccagacgtaggtttgtgtaaaagctcagttgtagcttatttcttgtaccttgcagcatgttctacgcatccagtccttaaggggtttactctacaaactgtgcacctgcgtgtaagggtcattagcaataagatagaaaattgagcaagtttataccataattttgtagaaaaaggaatctgctcaattccatatttcatccatgaaaactctgcaatacgggcagtttctaggaataaataaaaaggagatgcaaaccattgtagatgtcttctgtgggatgtgcccgatgcgtgcatcatcgtctttgattccaggtacttcataaagataaaattaaattc

SEQ ID NO: 47 - DNA, 1524 bp, Mus musculus
  misc_feature (1)..(1524): Mouse RBPJ CDS >NM_001277116.1242-1765 recombination signal binding protein for immunoglobulin kappa J region (Rbpj), transcript variant 4, mRNA [Mus musculus]
atgtggttgctgggatttgaactcctgaccttcagaagagcagtcgggtgctcttacccattgagccatctcaccagcccgaagtttggtgagcggcctccacccaaacgactcactagggaagctatgcgaaattatttaaaagaacgaggggatcaaacagtgctcattcttcatgcaaaagttgcacagaagtcttacggaaatgaaaaacgatttttttgccctcctccttgtgtgtatcttatgggcagtggttggaagaaaaaaaaagagcaaatggaacgagatggatgttctgaacaagagtctcaaccctgtgcgtttattggaataggaaacagtgaccaagaaatgcagcagctgaacttggaagggaagaactactgtacagccaaaacattgtacatatctgattcagacaagagaaaacatttcatgttgtctgtaaagatgttctatggcaacagcgatgacattggtgtgttcctcagcaagcggataaaggtcatctccaaaccctccaaaaagaagcagtcactgaagaatgctgacttgtgcattgcttcaggaacgaaggtggcactgttcaatcgccttcggtcccagacagttagtaccaggtacctgcatgtagaaggagggaatttccacgccagttcacaacagtggggagcattttacatccatctcttggacgacgacgagtcggaaggagaggagttcacagttagagatggctacatccattacgggcagactgtcaagcttgtgtgctcagtgactggcatggcactcccaagattgataattaggaaagttgataagcagacggcattactggatgcagacgaccctgtatcacaactccacaaatgtgcattttaccttaaggatacagaaagaatgtacttgtgcctttctcaagaaagaataatccaatttcaggccactccatgtccaaaagaacaaaataaggaaatgataaacgatggagcttcctggacaatcattagcacagacaaggccgagtacacgttctatgagggaatgggccccgtccttgccccagtcacccctgtgcctgtcgtagaaagtcttcagttgaatggcggcggggacgtagcaatgcttgaacttacaggacaaaactttactccaaatttaagagtgtggtttggggatgtagaagccgaaacaatgtacagatgtggagagagcatgctctgtgtggtcccagacatttctgcattccgggaaggttggagatgggtccgccagccagtccaggttccagtgactttggtccgaaatgacggggtcatttactccaccagccttaccttcacctacacaccagagccagggccgaggccacactgcagcgctgcaggagcgattctcagagccaactccagccaagtgccctccaatgagtcaaacacaaacagcgaggggaattacacaaatgccagcacaaattctaccagtgtcacatcgtccacagcaaccgtggtgtcctaa

SEQ ID NO: 48 - protein, 507 aa, Mus musculus
  MISC_FEATURE (1)..(507): Mouse RBPJ Protein >NP_001264045.1 recombining binding protein suppressor of hairless isoform 4 [Mus musculus]
MWLLGFELLTFRRAVGCSYPLSHLTSPKFGERPPPKRLTREAMRNYLKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFYIHLLDDDESEGEEFTVRDGYIHYGQTVKLVCSVTGMALPRLIIRKVDKQTALLDADDPVSQLHKCAFYLKDTERMYLCLSQERIIQFQATPCPKEQNKEMINDGASWTIISTDKAEYTFYEGMGPVLAPVTPVPVVESLQLNGGGDVAMLELTGQNFTPNLRVWFGDVEAETMYRCGESMLCVVPDISAFREGWRWVRQPVQVPVTLVRNDGVIYSTSLTFTYTPEPGPRPHCSAAGAILRANSSQVPSNESNTNSEGNYTNASTNSTSVTSSTATVVS

(19) United States
(12) Patent Application Publication
(10) Pub. No.: US 2024/0139239 A1
(43) Pub. Date: May 2, 2024

(54) COMPOSITIONS FOR REPROGRAMMING CELLS INTO DENDRITIC CELLS TYPE 2 COMPETENT FOR ANTIGEN PRESENTATION, METHODS AND USES THEREOF

(71) Applicant: Asgard Therapeutics AB, Lund (SE)

(72) Inventors: Carlos Filipe Ribeiro Lemos Pereira, Moledo (PT); Cristiana Ferreira Pires, Palhaça (PT); Fábio Fiúza Rosa, Touria (PT); Luis Filijpe Henriques Oliveira, Silveirinha Pequena (PT)

(21) Appl. No.: 17/778,901

(22) PCT Filed: Nov. 25, 2020
(86) PCT No.: PCT/EP2020/083400

§ 371 Date: May 23, 2022

(30) Foreign Application Priority Data
Nov. 25, 2019 (PT) 115933
Aug. 7, 2020 (EP) 20190159.2

(51) Int. Cl. A61K 35/15; A61K 39/00; C07K 14/47; C12N 5/0784; C12N 15/86

(52) U.S. Cl. CPC A61K 35/15; A61K 39/4615; A61K 39/464452; C07K 14/4702; C12N 5/0639; C12N 15/86; C12N 2780/00043

(57) ABSTRACT

The present disclosure relates to compositions for reprogramming cells into conventional dendritic cells (cDC), particularly into cDC type 2 (hereinafter referred to as“cDC2” or“CD11b-positive dendritic cells”), methods and uses thereof. The present disclosure relates to the development of methods for making conventional dendritic cells With antigen presenting capacity from differentiated, multipotent or pluripotent stem cells by introducing and expressing isolated/synthetic transcription factors. More particularly, the disclosure provides methods for obtaining conventional dendritic cells (cDC), particularly cDC type 2 or CD11b-positive dendritic cells, by direct cell reprogramming With the surprisingly use of combinations of specific transcription factors.

### TECHNICAL FIELD

The present disclosure relates to compositions for reprogramming cells into conventional dendritic cells (cDC), particularly into cDC type 2 (hereinafter referred to as “cDC2” or “CD11b-positive dendritic cells”), methods and uses thereof.

The present disclosure relates to the development of methods for making conventional dendritic cells with antigen presenting capacity from differentiated, multipotent or pluripotent stem cells by introducing and expressing isolated/synthetic transcription factors. More particularly, the disclosure provides methods for obtaining conventional dendritic cells (cDC), particularly cDC type 2 or CD11b-positive dendritic cells, by direct cell reprogramming with the surprisingly use of combinations of specific transcription factors.

### BACKGROUND

Cellular reprogramming relies on rewiring the epigenetic and transcriptional network of one cell state to that of a different cell type. Transcription factor (TF)-overexpression experiments have highlighted the plasticity of adult somatic or differentiated cells, providing new technologies to generate any desired cell type. Through forced expression of TFs, it is possible to reprogram somatic or differentiated cells into induced pluripotent stem cells (iPSCs) that are remarkably similar to embryonic stem cells (Takahashi et al., 2007; Takahashi & Yamanaka, 2006). Alternatively, a somatic cell can also be directly converted into another specialized cell type (Pereira, Lemischka, & Moore, 2012). Direct lineage conversion has proven successful to reprogram mouse and human fibroblasts into several cell types, such as neurons, cardiomyocytes and hepatocytes, using TFs specifying the target-cell identity (Xu, Du, & Deng, 2015). Direct cell conversions were also demonstrated in the hematopoietic system, where forced expression of TFs induced a macrophage fate in B cells and fibroblasts (Xie, Ye, Feng, & Graf, 2004) and the direct reprogramming of mouse fibroblasts into clonogenic hematopoietic progenitors was achieved with Gata2, Gfi1b, cFos and Etv6 (Pereira et al., 2013). These four TFs induce a dynamic, multi-stage hemogenic process that progresses through an endothelial-like intermediate, recapitulating developmental hematopoiesis in vitro (Pereira et al., 2016).

Reprogrammed cells are very promising therapeutic tools for regenerative medicine, and cells obtained by differentiation of iPSCs are already being tested in clinical studies.

Cellular reprogramming strategies have highlighted the flexibility of cell fates with the possibility to use cell-type-specific TFs to convert somatic cells into pluripotency. Direct lineage conversions of one differentiated cell-type into another have also been demonstrated and explored for elucidating cell biology mechanisms and for regenerative medicine purposes. Recently, it has been demonstrated that antigen presenting dendritic cells can be reprogrammed from unrelated cell-types by a small combination of TFs. Classically, it is said that a myeloid DC committed progenitor gives rise to the functionally different DC subsets: conventional DCs (cDCs), which are professional antigen presenting cells (APCs), and plasmacytoid DCs (pDCs). cDCs drives antigen-specific immune responses, while pDCs are professional producers of type I interferons during viral infection. However, the timing and exact mechanisms regulating the divergence of the different subsets during DC development are still to be established.

DCs are a class of bone-marrow-derived cells arising from lympho-myeloid hematopoiesis that scan the organism for pathogens, forming an essential interface between the innate immune system and the activation of adaptive immunity. DCs act as professional APCs capable of activating T cell responses by displaying peptide antigens complexed with the major histocompatibility complex (MHC) on the surface, together with all the necessary soluble and membrane associated co-stimulatory molecules. DCs induce primary immune responses, potentiate the effector functions of previously primed T-lymphocytes and orchestrate communication between innate and adaptive immunity. DCs are found in most tissues, where they continuously sample the antigenic environment and use several types of receptors to monitor for invading pathogens. In a steady state, and at an increased rate upon detection of pathogens, sentinel DCs in non-lymphoid tissues migrate to the lymphoid organs where they present to T cells the antigens they have collected and processed. The phenotype acquired by the T cell depends on the context of antigen presentation. If the antigen is derived from a pathogen, or damaged self, DCs will receive danger signals, becoming activated and subsequently stimulating T cells to become effectors, necessary to provide protective immunity.

An important aspect of the control of immune responses is the existence of several different types of DCs, each specialized to respond to particular pathogens and to interact with specific subsets of T cells. In this context, three major DC subsets arise: plasmacytoid DC (pDC), myeloid/conventional DC1 (cDC1) and myeloid/conventional DC2 (cDC2). This expands the flexibility of the immune system to react appropriately to a wide range of different pathogens and danger signals.

cDC2 are characterized by CD11b surface expression and are specialized in MHC-II presentation directing naïve CD4⁺ T cell polarization toward helper Th2 and Th17 (Plantinga et al., 2013). While Th2-associated cytokines (IL-4, IL-5, IL-9 and IL-13) mediate responses related to protection against extracellular parasites (Mosmann & Coffman, 1989) and induce allergy and hypersensitivity reactions (Kopf et al., 1993; Zhu & Paul, 2008), Th17 are associated with immune responses against extracellular bacteria and fungi, also inducing many autoimmune diseases (Weaver, Harrington, Mangan, Gavrieli, & Murphy, 2006). In tumors, cDC2s are known to complement cDC1 by being involved in antigen presentation on MHC-II to CD4⁺ T cells in tumor-draining lymph nodes (Merad, Sathe, Helft, Miller, & Mortha, 2013). In one study, antigen presentation of tumor-derived cDC2 was proven to drive conversion of tumor-associated macrophages towards an anti-tumor phenotype on a Th17-dependent matter (Laoui et al., 2016). cDC2 also contribute to downregulation of effector T cells by priming regulatory T cells (Treg) which are vital in the maintenance of self-tolerance by destroying self-reactive CD4⁺ T cells (Merad et al., 2013) and negatively regulating immune responses (Sakaguchi, 2004).

Additional cDC2 characteristic markers include CD11 b, Sirpα, CD4, and ESAM. Due to cDC2s' inherent heterogeneity, some specific surface markers characterize particular subsets. Recent findings have identified two distinct subsets of cDC2 defined by different transcriptional regulators and distinct immunological functions (Brown et al., 2019). While the cDC2A is an anti-inflammatory subset defined as Tbet-dependent and characterized by surface expression of Esam and Clec4a4, cDC2B consist of a rather pro-inflammatory, RORγt-defined subset expressing Clec10a and Clec12a markers.

The ability of DCs to induce adaptive immunity has boosted research on DC-vaccination strategies for bacterial, viral and parasitic pathogens and immunotherapy, namely in cancer. In fact, clinical trials are ongoing which are utilizing DC-mediated immunotherapy for several tumor types, including solid and hematological tumors (Datta et al., 2014). However, the clinical outcome has been inconsistent, probably associated with the variable efficiency of in vitro-generated DCs: autologous monocytes can be differentiated in vitro into less efficient DCs, and hematopoietic progenitors are isolated in very low numbers. In addition, these precursor cells are commonly compromised in cancer-bearing patients, resulting in the generation of dysfunctional DCs (Datta et al., 2014; Subklewe et al., 2014). Cancer evasion mechanisms may also underlie the lack of consistent therapeutic advantages in DC-based immunotherapies. During tumor progression, cancer cells exploit several immunological processes to escape immune surveillance. These adaptations, together with cancer antigen heterogeneity, prevent the recognition of tumor antigens by the immune system and are consequently responsible for the reduced immunogenicity of tumor cells and current immunotherapies.

The generation of APCs by direct reprogramming opens new opportunities to better understand DC specification and cellular identity, contributing to a more efficient control of immune responses using autologous-engineered cells.

Document EP 3 385 373 relates to compositions, nucleic acid constructs, methods and kits thereof for cell induction or reprogramming cells to the DC state or APC state based, in part, on the surprisingly effect of novel use and combinations of TFs that allow induction or reprogramming of differentiated or undifferentiated cells into DCs or APCs.

The generated reprogrammed cells described in document EP3385373 mainly recapitulate surface marker expression, antigen presentation, cytokine release and T-cell activation features specifically of the cDC1 subset of DCs. Phenotype features of other DC subsets were not described.

Antigen-presenting cells (APCs) are a heterogeneous group of immune cells that mediate the cellular immune response by processing and presenting antigens for recognition by certain lymphocytes such as T cells. Classical APCs include dendritic cells, macrophages, Langerhans cells and B cells.

DCs provide a crucial link between the external environment and the adaptive immune system through their ability to capture, process and present antigens to T cells, targeting them to different types of immune responses or inducing tolerance responses.

Phenotypic criteria allow the classification of mouse DCs into different subpopulations characterized by the expression of distinct surface markers. Conventional DCs (cDCs) in lymphoid tissues are traditionally sub-divided into cDC1s and cDC2 subpopulations. Different DC subsets are involved in specific recognition of certain pathogens and/or regulate different immune responses. While cDC1s are associated with priming of Th1 responses, important in promoting tumor clearance, cDC2 subsets have been associated with Th1, Th2, Th17 (immunity) and Treg (tolerance) responses.

Document EP 3 385 373 relates to compositions, nucleic acid constructs, methods and kits thereof for cell induction or reprogramming cells to the DC state or

APC state, based, in part, on the surprising effect of novel use and combinations of TFs that allow the induction or reprogramming of differentiated or undifferentiated cells into DCs or APCs, more specifically cDC1s.

Presently, DC-based immunotherapies rely on autologous DC precursors: either monocytes, which are associated with the production of less-efficient DCs, or hematopoietic progenitors, which are isolated in very low numbers. Additionally, these precursor cells are commonly compromised in cancer-bearing patients, resulting in the generation of dysfunctional DCs. Non-hematopoietic cell-types such as fibroblast, on the other hand, are usually not affected. Given the fundamental role of DCs as APCs bridging the innate and adaptive immune systems, there remains a clinical need to find alternative strategies to generate functional DCs to prime antigen-specific immune responses.

### SUMMARY

The induced DCs or APCs generated by direct reprograming of the present disclosure surprisingly recapitulate a phenotype regarding surface marker expression, cytokine secretion and antigen presentation in MHC-II molecules of cDC2 subset of DCs.

These facts are disclosed in order to illustrate the technical problem addressed by the present disclosure.

The present subject matter identifies several isolated or synthetic TFs that surprisingly reprogram or induce differentiated cells, multipotent or pluripotent stem cells into antigen presenting dendritic cells, more specifically cDC2s, in vitro, ex vivo or in vivo.

In an aspect, the present disclosure comprises a composition comprising a combination of at least two transcription factors encoded by an isolated or synthetic sequence at least 90% identical to and selected from a list consisting of: PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11), PRDM1 (SEQ. ID. 13, SEQ. ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. 25, SEQ. ID. 26, SEQ. ID. 28, SEQ. ID. 29), TGIF1 (SEQ. ID. 31, SEQ. ID. 32, SEQ. ID. 34, SEQ. ID. 35); or at least two isolated or synthetic transcription factors at least 90% identical from a sequence selected from a list consisting of: PU.1 (SEQ. ID. 3, SEQ. ID. 6), IRF4 (SEQ. ID. 9, SEQ. ID. 12), PRDM1 (SEQ. ID. 15, SEQ. ID. 18), IRF2 (SEQ. ID. 21, SEQ. ID. 24), POU2F2 (SEQ. ID. 27, SEQ. ID. 30), TGIF1 (SEQ. ID. 33, SEQ. ID. 36), and mixtures thereof; for reprogramming stem cells or differentiated cells, or mixtures thereof into conventional dendritic cells type 2 (cDC2) or CD11b-positive dendritic cells.

In an aspect, the present disclosure comprises a composition comprising a combination of at least three isolated or synthetic transcription factors, the first and second being isolated and synthetic PU.1 and IRF4 transcription factors at least 90% identical to a sequence of: PU.1 (SEQ. ID. 3, SEQ. ID. 6) and IRF4 (SEQ. ID. 9, SEQ.

ID. 12), and the third being an isolated or synthetic transcription factor at least 90% identical to a sequence selected from the group consisting of: PRDM1 (SEQ. ID. 15, SEQ. ID. 18), IRF2 (SEQ. ID. 21, SEQ. ID. 24), POU2F2 (SEQ. ID. 27, SEQ. ID. 30), TGIF1 (SEQ. ID. 33, SEQ. ID. 36), RBPJ (SEQ. ID. 45, SEQ. ID. 48) and RELB (SEQ. ID. 39, SEQ. ID. 42);

for use in reprogramming stem cells or differentiated cells, or mixtures thereof into conventional dendritic cells type 2 (cDC2) or CD11b-positive dendritic cells.

By variant as used herein is meant a sequence with 60%, 61%, 62%, 30 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the DNA encoded sequences of the present disclosure.

In a further embodiment, the present disclosure comprises a composition wherein at least two transcription factors
encoded by an isolated or synthetic sequence at least 90% identical to and selected from the group consisting of: PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11), PRDM1 (SEQ. ID. 13, SEQ.

ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. 25, SEQ. ID. 26, SEQ. ID. 28, SEQ. ID. 29), TGIF1 (SEQ. ID. 31, SEQ. ID. 32, SEQ. ID. 34, SEQ. ID. 35) or at least two isolated or synthetic transcription factors at least 90% identical from a sequence selected from the group consisting of: PU.1 (SEQ. ID. 3, SEQ. ID. 6), IRF4 (SEQ. ID. 9, SEQ. ID. 12), PRDM1

(SEQ. ID. 15, SEQ. ID. 18), IRF2 (SEQ. ID. 21, SEQ. ID. 24), POU2F2 (SEQ. ID. 27, SEQ. ID. 30), TGIF1 (SEQ. ID. 33, SEQ. ID. 36) and mixtures thereof;
for use in reprogramming stem cells or differentiated cells, or mixtures thereof into conventional dendritic cells type 2 (cDC2),
with the proviso that a combination of at least two isolated or synthetic transcription factors consisting of: PU.1 (SEQ. ID. 1—SEQ. ID. 6), IRF4 (SEQ. ID. 7—SEQ. ID. 12) is excluded.

In an embodiment, the present disclosure comprises a composition wherein at least two transcription factors encoded by an isolated or synthetic sequence at least 90% identical to and selected from the group consisting of: PU.1 (SEQ. ID. 1,

SEQ. ID. 2, SEQ. ID. 4, SEQ. ID. 5), IRF4 (SEQ. ID. 7, SEQ. ID. 8, SEQ. ID. 10, SEQ. ID. 11), PRDM1 (SEQ. ID. 13, SEQ. ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. 25, SEQ. ID. 26, SEQ. ID. 28, SEQ. ID. 29), TGIF1 (SEQ. ID. 31, SEQ. ID. 32, SEQ. ID. 34, SEQ. ID. 35), RBPJ (SEQ. ID. 43, SEQ. ID. 44, SEQ. ID. 46, SEQ. ID. 47) and RELB (SEQ. ID. 37,

SEQ. ID. 38, SEQ. ID. 40, SEQ. ID. 41) or at least two isolated or synthetic transcription factors at least 90% identical from a sequence selected from the group consisting of: PU.1 (SEQ. ID. 3, SEQ. ID. 6), IRF4 (SEQ. ID. 9, SEQ. ID. 12), PRDM1 (SEQ. ID. 15, SEQ. ID. 18), IRF2 (SEQ. ID. 21, SEQ. ID. 24), POU2F2 (SEQ. ID. 27, SEQ. ID. 30), TGIF1 (SEQ. ID. 33, SEQ. ID. 36)), RBPJ (SEQ. ID. 45, SEQ. ID. 48) and RELB (SEQ. ID. 39, SEQ. ID. 42) and mixtures thereof;
for use in reprogramming stem cells or differentiated cells into conventional dendritic cells type 2 (cDC2) or CD11b-positive dendritic cells.

In one embodiment, the present disclosure comprises the composition for use as previously described herein, wherein the transcription factors individually are encoded by polynucleotides being at least 90% identical to the following sequences: PU.1 (SEQ. ID. 1, SEQ. ID. 2,SEQ. ID. 4, SEQ. ID. 5), IRF4 (SEQ. ID. 7, SEQ. ID. 8, SEQ. ID. 10, SEQ. ID. 11), PRDM1 (SEQ. ID. 13, SEQ. ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. SEQ. ID. 26, SEQ. ID. 28, SEQ. ID. 29), TGIF1 (SEQ. ID. 31, SEQ. ID. 32, SEQ. ID. 34, SEQ. ID. 35), RBPJ (SEQ. ID. 43, SEQ. ID. 44, SEQ. ID. 46, SEQ. ID. 47), RELB (SEQ. ID. 37, SEQ. ID. 38, SEQ. ID. 40, SEQ. ID. 41).

In an embodiment, the present disclosure comprises a combination of at least two transcription factors encoded by an isolated or synthetic sequence at least 95% identical to and selected from a list consisting of: PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11), PRDM1 (SEQ. ID. 13, SEQ. ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. 25, SEQ. ID. 26, SEQ. ID. 28, SEQ. ID. 29), TGIF1 (SEQ. ID. 31, SEQ. ID. 32, SEQ. ID. 34, SEQ. ID. 35) or at least two isolated or synthetic transcription factors at least 95% identical from a sequence selected from a list consisting of: PU.1 (SEQ. ID. 3, SEQ. ID. 6), IRF4 (SEQ. ID. 9, SEQ. ID. 12), PRDM1 (SEQ. ID. 15, SEQ. ID. 18), IRF2 (SEQ. ID. 21, SEQ. ID. 24), POU2F2 (SEQ. ID. 27, SEQ. ID. 30), TGIF1 (SEQ. ID. 33, SEQ. ID. 36) and mixtures thereof.

In one embodiment, the present disclosure comprises the composition for use as described previously herein, wherein the transcription factors individually are encoded by polynucleotide at least 95% identical to the following sequences: PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11), PRDM1 (SEQ. ID. 13, SEQ. ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. 25, SEQ. ID. 26, SEQ. ID. 28, SEQ. ID. 29), TGIF1 (SEQ. ID. 31, SEQ. ID.32, SEQ. ID. 34, SEQ. ID. 35), RBPJ (SEQ. ID. 43, SEQ. ID. 44, SEQ. ID. 46, SEQ. ID. 47), RELB (SEQ. ID. 37, SEQ. ID. 38, SEQ. ID. 40, SEQ. ID. 41) or the isolated or synthetic transcription factors individually are at least 95% identical to the following sequences: PU.1 (SEQ. ID. 3, SEQ. ID. 6), IRF4 (SEQ. ID. 9, SEQ. ID. 12), PRDM1 (SEQ. ID. 15, SEQ. ID. 18), IRF2 (SEQ. ID. 21, SEQ. ID. 24), POU2F2 (SEQ. ID. 27, SEQ. ID. 30), TGIF1 (SEQ. ID. 33, SEQ. ID. 36), RBPJ (SEQ. ID. 45, SEQ. ID. 48), RELB (SEQ. ID. 39, SEQ. ID. 42).

In a further embodiment, the present disclosure comprises a combination of at least two transcription factors that is selected from the following isolated or synthetic encoded combinations or from the following proteins:
PU.1 (SEQ. ID. 1-SEQ. ID. 6) and IRF4 (SEQ. ID. 7SEQ. ID.12);
PU.1 (SEQ. ID. 1-SEQ. ID. 6) and PRDM1 (SEQ. ID. 13-SEQ. ID. 18);
IRF4 (SEQ. ID. 7-SEQ. ID.12), and PRDM1 (SEQ. ID. 13-SEQ. ID. 18);
PU.1 (SEQ. ID. 1-SEQ. ID. 6) and IRF2 (SEQ. ID. 19SEQ. ID. 24);
PU.1 (SEQ. ID. 1-SEQ. ID. 6) and POU2F2 (SEQ. ID. 25SEQ. ID. 30);
PU.1 (SEQ. ID. 1-SEQ. ID. 6) and TGIF1 (SEQ. ID. 31SEQ. ID.36);
IRF4 (SEQ. ID. 7-SEQ. ID.12) and IRF2 (SEQ. ID. 19SEQ. ID. 24);
IRF4 (SEQ. ID. 7-SEQ. ID.12) and POU2F2 (SEQ. ID. 25SEQ. ID. 30);
IRF4 (SEQ. ID. 7-SEQ. ID.12) and TGIF1 (SEQ. ID. 31SEQ. ID.36);
PRDM1 (SEQ. ID. 13-SEQ. ID. 18) and IRF2 (SEQ. ID. 19SEQ. ID. 24);
PRDM1 (SEQ. ID. 13-SEQ. ID. 18) and POU2F2 (SEQ. ID. 25SEQ. ID. 30);
PRDM1 (SEQ. ID. 13-SEQ. ID. 18) and TGIF1 (SEQ. ID. 31SEQ. ID.36);
PU.1 (SEQ. ID. 1-SEQ. ID. 6) and IRF4 (SEQ. ID. 7SEQ. ID. 12);
IRF2 (SEQ. ID. 19-SEQ. ID. 24) and POU2F2 (SEQ. ID. 25SEQ. ID. 30);
IRF2 (SEQ. ID. 19-SEQ. ID. 24) and TGIF1 (SEQ. ID. 31SEQ. ID.36);
PU.1 (SEQ. ID. 1-SEQ. ID. 6) and POU2F2 (SEQ. ID. 25SEQ. ID. 30);
POU2F2 (SEQ. ID. 25-SEQ. ID. 30) and TGIF1 (SEQ. ID. 31SEQ. ID.36);
PU.1 (SEQ. ID. 1-SEQ. ID. 6) and TGIF1 (SEQ. ID. 31-SEQ. ID.36);
PU.1 (SEQ. ID. 1-SEQ. ID. 6), IRF4 (SEQ. ID. 7-SEQ. ID. 12), PRDM1 (SEQ. ID. 13-SEQ. ID. 18), IRF2 (SEQ. ID. 19-SEQ. ID. 24), POU2F2 (SEQ. ID. 25-SEQ. ID. 30) and TGIF1 (SEQ. ID. 31-SEQ. ID. 36);
PU.1 (SEQ. ID. 1-SEQ. ID. 6), IRF4 (SEQ. ID. 7-SEQ. ID. 12) and RBPJ (SEQ. ID. 43-SEQ. ID. 48);
PU.1 (SEQ. ID. 1-SEQ. ID. 6), IRF4 (SEQ. ID. 7-SEQ. ID. 12) and RELB (SEQ. ID. 37-SEQ. ID. 42);
or mixtures thereof.

In one embodiment, the present disclosure comprises the composition for use as previously described, wherein the combination of transcription factors is selected from the following combinations:
PU.1, IRF4 and PRDM1;
PU.1, IRF4 and IRF2;
PU.1, IRF4 and POU2F2;
PU.1, IRF4 and TGIF1;
PU.1, IRF4 and RBPJ; and
PU.1, IRF4 and RELB.

In an embodiment, the composition of the present disclosure may comprise at least three transcription factors encoded by an isolated or synthetic sequence at least 90% identical to a sequence selected from the group consisting of or from the following proteins: PU.1 (SEQ. ID. 1-SEQ. ID. 6), IRF4 (SEQ. ID. 7-SEQ. ID. 12), PRDM1 (SEQ. ID. 13-SEQ. ID. 18), IRF2 (SEQ. ID. 19-SEQ. ID. 24), POU2F2 (SEQ. ID. 25-SEQ. ID. 30), TGIF1 (SEQ. ID. 31-SEQ. ID.36) and mixtures thereof.

In an embodiment, the present disclosure relates to the composition as described herein, wherein the combination of transcription factors is: PU.1, IRF4, PRDM1 or PU.1, IRF4 and IRF2.

In a further embodiment, the composition of the present disclosure may comprise the combination of transcription factors selected from the following isolated or synthetic protein or from the isolated or synthetic encoded combinations of:

PU.1 (SEQ. ID. 1-SEQ. ID. 6), IRF4 (SEQ. ID. 7-, SEQ. ID. 12), and PRDM1 (SEQ. ID. 13-SEQ. ID. 18);
PU.1 (SEQ. ID. 1-SEQ. ID. 6), IRF4 (SEQ. ID. 7-, SEQ. ID. 12), and IRF2 (SEQ. ID. 19-, SEQ. ID. 24);
PU.1 (SEQ. ID. 1-SEQ. ID. 6), IRF4 (SEQ. ID. 7-SEQ. ID. 12) and POU2F2 (SEQ. ID. 25-SEQ. ID. 30);
PU.1 (SEQ. ID. 1-SEQ. ID. 6), IRF4 (SEQ. ID. 7-SEQ. ID. 12) and TGIF1 (SEQ. ID. 31-, SEQ. ID. 36);
PU.1 (SEQ. ID. 1-SEQ. ID. 6), IRF4 (SEQ. ID. 7-SEQ. ID. 12), PRDM1 (SEQ. ID. 13-SEQ. ID. 18);
IRF2 (SEQ. ID. 19-SEQ. ID. 24), POU2F2 (SEQ. ID. 25-SEQ. ID. 30) and TGIF1 (SEQ. ID. 31-SEQ. ID. 36);
PU.1 (SEQ. ID. 1-SEQ. ID. 6), IRF4 (SEQ. ID. 7-SEQ. ID. 12) PRDM1 (SEQ. ID. 13-SEQ. ID. 18), IRF2 (SEQ. ID. 19-SEQ. ID. 24), POU2F2 (SEQ. ID. 25-SEQ. ID. 30) and TGIF1 (SEQ. ID. 31-SEQ. ID. 36);
PU.1 (SEQ. ID. 1-SEQ. ID. 6), IRF4 (SEQ. ID. 7-SEQ. ID. 12) and RBPJ (SEQ. ID. 43 - SEQ. ID. 48);
PU.1 (SEQ. ID. 1-SEQ. ID. 6), IRF4 (SEQ. ID. 7-SEQ. ID. 12) and RELB (SEQ. ID. 37 - SEQ. ID. 42);
or mixtures thereof.

In an embodiment, the combination of isolated or synthetic transcription factors is: PU.1 (SEQ. ID. 1-SEQ. ID. 6), IRF4 (SEQ. ID. 7-SEQ. ID. 12) and PRDM1 (SEQ. ID. 13-SEQ. ID. 18).

In one embodiment, the combination of isolated or synthetic transcription factors is: PU.1, IRF4 and PRDM1.

In a further embodiment, the combination of isolated or synthetic transcription factors is: PU.1 (SEQ. ID. 1-SEQ. ID. 6), IRF4 (SEQ. ID. 7-SEQ. ID. 12) and PRDM1 (SEQ. ID. 13-SEQ. ID. 18) or PU.1 (SEQ. ID. 1-SEQ. ID. 6), IRF4 (SEQ. ID. 7-SEQ. ID. 12) and IRF2 (SEQ. ID. 19-SEQ. ID. 24).

In another embodiment, the combination of isolated or synthetic transcription factors is: PU.1, IRF4 and PRDM1 or PU.1, IRF4 and IRF2.

In an embodiment, the composition of the present disclosure may comprise stem cells or differentiated cells selected from the group consisting of: pluripotent stem cell, multipotent stem cell, differentiated cell, fibroblast, tumor cell, cancer cell, and mixtures thereof.

In an embodiment, a cell may be selected from the group consisting of: pluripotent stem cell, multipotent stem cell, differentiated cell, fibroblast, tumor cell, cancer cell, and mixtures thereof.

In a further embodiment, the cell may be selected from the group consisting of: tumor cell, cancer cell, and mixtures thereof.

In an embodiment, the antigen may be a cancer antigen, a self-antigen, an allergen, an antigen from a pathogenic and/or infectious organism.

In an embodiment, the composition of the present disclosure may be

used in veterinary or human medicine, in particular in immunotherapy, or in autoimmune diseases, immunodeficiency, or in neurodegenerative or ageing diseases, or in cancer or in infectious diseases, or as a drug screening.

In a further embodiment, the pluripotent stem cell, multipotent stem cell or differentiated cell is a mammalian pluripotent stem cell, multipotent stem cell or differentiated cell, in particular a mouse or a human cell.

One aspect of the present disclosure relates to a construct or a vector encoding at least the combination of two isolated or synthetic transcription factors of the present disclosure, preferably the encoded isolated or synthetic combination of three transcription factors.

Another aspect of the present disclosure relates to a construct or vector encoding the combination of transcription factors as described herein.

In a further embodiment, the present disclosure comprises a construct or the vector wherein the combination of three isolated or synthetic transcription factors is in the following sequential order from 5′ to 3′:
PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11) and PRDM1 (SEQ. ID. 13, SEQ. ID. 14, SEQ. ID. 16, SEQ. ID. 17); PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11), and IRF2 (SEQ. ID. 19, SEQ. ID. 20, SEQ. ID. 22, SEQ. ID. 23) PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11) and POU2F2 (SEQ. ID. 25, SEQ. ID. 26, SEQ. ID. 28, SEQ. ID. 29);
PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11) and TGIF1 (SEQ. ID. 31, SEQ. ID. 32, SEQ. ID. 34, SEQ. ID. 35);
PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11), PRDM1 (SEQ. ID. 13, SEQ. ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. 25, SEQ. ID. 26, SEQ. ID. 28, SEQ. ID. 29) and TGIF1 (SEQ. ID. 31, SEQ. ID.32, SEQ. ID. 34, SEQ. ID. 35); PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11) and RBPJ (SEQ. ID. 43, SEQ. ID. 44, SEQ. ID. 46, SEQ. ID. 47);
PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11) and RELB (SEQ. ID. 37, SEQ. ID. 38, SEQ. ID. 40, SEQ. ID. 41).

In a further embodiment, the present disclosure comprises a construct or the vector wherein the combination of encoded transcription factors is in the following sequential order from 5′ to 3′:
PU.1, IRF4 and PRDM1;
PU.1, IRF4 and IRF2;
PU.1, IRF4 and POU2F2;
PU.1, IRF4 and TGIF1;
PU.1, IRF4 and RBPJ; or
PU.1, IRF4 and RELB.

In a further embodiment, the present disclosure comprises a vector wherein the vector is a viral vector; in particular a retroviral, adenoviral, lentiviral, herpes viral, pox viral, or adeno-associated viral vector.

In an embodiment, the vector or construct is synthetic mRNA, naked alphavirus RNA replicons or naked flavivirus RNA replicons.

In an aspect of the present disclosure, the disclosure relates to one or more vectors comprising at least three polynucleotide sequences, encoding at least three transcription factors, the first and second being PU.1 and IRF4 and the third 30 being selected from the group consisting of: PRDM1, IRF2, POU2F2, RBPJ, RELB and TGIF1, for use in reprogramming of stem cells or differentiated cells into conventional dendritic cells type 2 (cDC2) or CD11b-positive dendritic cells.

In one embodiment, the present disclosure relates to the one or more vectors, wherein the transcription factors individually are encoded by polynucleotides being at least 90% identical to the sequences selected from the group consisting of: PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11), PRDM1 (SEQ. ID. 13, SEQ. ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. 25, SEQ. ID. 26, SEQ. ID. 28, SEQ. ID. 29), TGIF1 (SEQ. ID. 31, SEQ. ID.32, SEQ. ID. 34, SEQ. ID. 35), RELB (SEQ. ID. 37, SEQ. ID. 38, SEQ. ID. 40, SEQ. ID. 41) and RBPJ (SEQ. ID. 43, SEQ. ID. 44, SEQ. ID. 46, SEQ. ID. 47).

In a further embodiment, the present disclosure comprises one or more vectors wherein the combination of encoded transcription factors is selected from the following combinations:
PU.1, IRF4 and PRDM1;
PU.1, IRF4 and IRF2;
PU.1, IRF4 and POU2F2;
PU.1, IRF4 and TGIF1;
PU.1, IRF4 and RBPJ; or
PU.1, IRF4 and RELB.

In an aspect, the present disclosure relates to one or more vectors comprising at least three polynucleotide sequences encoding at least three transcription factors, wherein the transcription factors are PU.1, IRF4 and PRDM1.

In one embodiment, the present disclosure comprises one or more vectors wherein the one or more vectors are viral vectors; in particular retroviral, adenoviral, lentiviral, herpes viral, pox viral, paramyxoviral, rabdoviral, alphaviral, flaviviral or adeno-associated viral vectors.

In one embodiment, the present disclosure comprises one or more vectors wherein the one or more vectors are synthetic mRNA, naked alphavirus RNA replicons or naked flavivirus RNA replicons.

In one embodiment, the present disclosure comprises one or more vectors wherein the cell is selected from the group consisting of: pluripotent stem cell, multipotent stem cell, differentiated cell, tumor cell, cancer cell and mixtures thereof.

In one embodiment, the present disclosure comprises one or more vectors for use in veterinary or human medicine, particularly in immunotherapy, or in the treatment or therapy of neurodegenerative diseases, or in autoimmune diseases, immunodeficiency, or in the treatment or therapy of cancer or in the treatment or therapy of an infectious disease; intradermal and transdermal therapies; in immunotherapy, or in neurodegenerative or ageing diseases, or in cancer or in infectious diseases, as a drug screening; or for use in the treatment, therapy or diagnosis of a central and peripheral nervous system disorder, neoplasia in particular cancer, namely solid or hematological tumors, immunological diseases, in particular autoimmune diseases, hypersensitivities, or immunodeficiency; of fungal, viral, chlamydial, bacterial, nanobacterial or parasitic infectious diseases; of HIV, infection with SARS coronavirus, Asian flu virus, herpes simplex, herpes zoster, hepatitis, or viral hepatitis.

Another aspect of the present disclosure relates to a method for reprogramming or inducing a stem cell or a differentiated cell into a conventional dendritic cell type 2, comprising the following steps:
transducing a cell selected from the group consisting of: stem cell or a differentiated cell, and mixtures thereof,
with one or more vectors comprising at least two nucleic acid sequences encoding a sequence at least 90% identical, preferably at least 95% identical, to a sequence from the group consisting of PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11), PRDM1 (SEQ. ID. 13, SEQ. ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. 25, SEQ. ID. 26, SEQ. ID. 28, SEQ. ID. 29), TGIF1 (SEQ. ID. 31, SEQ. ID.32, SEQ. ID. 34, SEQ. ID. 35), RBPJ (SEQ. ID. 43, SEQ. ID. 44, SEQ. ID. 46, SEQ. ID. 47), and RELB (SEQ. ID. 37, SEQ. ID. 38, SEQ. ID. 40, SEQ. ID. 41), and mixtures thereof;
culturing the transduced cell in a cell media that supports growth of dendritic cells or antigen-presenting cells.

Another aspect of the present disclosure relates to a method for reprogramming or inducing a stem cell or a differentiated cell into a conventional dendritic cell type 2, comprising the following steps:
transducing a cell selected from the group consisting of: a stem cell or a differentiated cell, and mixtures thereof, with one or more vectors encoding at least three transcription factors the first and second being PU.1 and IRF4 and the third being selected from the group consisting of PRDM1, IRF2, POU2F2, TGIF1, RELB and RBPJ; and mixtures thereof;
culturing the transduced cell in a cell media that supports growth of dendritic cells or antigen-presenting cells.

In one embodiment, the present disclosure relates to the method as described herein, wherein the transduced cells are cultured during at least 2 days, preferably at least 5 days, more preferably at least 8 days, even more preferably at least 9 days, more preferably at least 10 days.

In a further embodiment, the combination of sequences may be:
PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11) and PRDM1 (SEQ. ID. 13, SEQ. ID. 14, SEQ. ID. 16, SEQ. ID. 17);
PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11) and IRF2 (SEQ. ID. 19, SEQ. ID. 20, SEQ. ID. 22, SEQ. ID. 23);
PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11) and POU2F2 (SEQ. ID. 25, SEQ. ID. 26, SEQ. ID. 28, SEQ. ID. 29);
PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11) and TGIF1 (SEQ. ID. 31, SEQ. ID.32, SEQ. ID. 34, SEQ. ID. 35);
PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11) and RBPJ (SEQ. ID. 43, SEQ. ID. 44, SEQ. ID. 46, SEQ. ID. 47);
PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11) and RELB (SEQ. ID. 37, SEQ. ID. 38, SEQ. ID. 40, SEQ. ID. 41).

In an embodiment, the present disclosure comprises a construct or a vector of the present disclosure, wherein the sequence selected from said group is a combination of PU.1 and IRF4.

In an aspect, the present disclosure comprises a method for reprogramming or inducing a stem cell or a differentiated cell into a conventional dendritic cell type 2, comprising the following steps: transducing a cell selected from the group consisting of: stem cell or a differentiated cell, and mixtures thereof, with one or more vectors comprising at least two nucleic acid sequences encoding a sequence at least 90% identical, preferably at least 95% identical, to a sequence from the group consisting of PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11), PRDM1 (SEQ. ID. 13, SEQ. ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. 25, SEQ. ID. 26, SEQ. ID. 28, SEQ. ID. 29), TGIF1 (SEQ. ID. 31, SEQ. ID. 32, SEQ. ID. 34, SEQ. ID. 35), RBPJ (SEQ. ID. 43, SEQ. ID. 44, SEQ. ID. 46, SEQ. ID. 47), and RELB (SEQ. ID. 37, SEQ. ID. 38, SEQ. ID. 40, SEQ. ID. 41),; and mixtures thereof; culturing the transduced cell in a cell media that supports growth of dendritic cells or antigen presenting cells.

In an embodiment, the combination of three isolated and synthetic transcription factors is in the following sequential order from 5′ to 3′: PU.1 (SEQ. ID. 1, SEQ. ID. 2, SEQ. ID. 4, SEQ. ID. 5)), IRF4 (SEQ. ID. 7, SEQ. ID. 8,

SEQ. ID. 10, SEQ. ID. 11), PRDM1 (SEQ. ID. 13, SEQ. ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. 25, SEQ. ID. 26, SEQ. ID. 28, SEQ. ID. 29), TGIF1 (SEQ. ID. 31, SEQ. ID. 32, SEQ. ID. 34, SEQ. ID. 35). The method includes but is not limited to culturing the cell transduced with a plurality of isolated and synthetic transcription factors during at least 2 days, preferably at least 5 days, more preferably at least 8 days, even more preferably at least 9 days, even more preferably at least 10 days.

In a further embodiment, the present disclosure comprises a method, wherein the transducing step further comprises at least one vector selected from the group consisting of: a nucleic acid sequence encoding IL-12; nucleic acid sequence encoding IL-4; a nucleic acid sequence encoding IFN-α; a nucleic acid sequence encoding IFN-β; a nucleic acid sequence encoding IFN-γ; a nucleic acid sequence encoding TNF; nucleic acid sequence encoding GM-CSF; nucleic acid sequence encoding siRNAs targeting IL-10 RNA , and mixtures thereof.

In an embodiment, the present disclosure comprises a method wherein transducing step further comprises at least one vector comprising nucleic acids encoding immunostimulatory cytokines.

In a further embodiment, the present disclosure comprises a method wherein the cell is selected from the group consisting of: pluripotent stem cell, or multipotent stem cell, or differentiated cell, and mixtures thereof.

In a further embodiment, the present disclosure comprises a method wherein the cell is a mammalian cell.

In a further embodiment, the present disclosure comprises a method wherein the pluripotent stem cell, multipotent stem cell, or differentiated cell, is selected from a group consisting of: an endoderm derived cell, a mesoderm derived cell, or an ectoderm derived cell, a multipotent stem cell including mesenchymal stem cell, a hematopoietic stem cell, an intestinal stem cell, a pluripotent stem cell and a cell line.

In an embodiment, the present disclosure comprises a method, wherein the cell is a non-human cell.

In a further embodiment, the present disclosure comprises a method, wherein the cell is a mouse cell.

In an embodiment, the present disclosure comprises a method, wherein the cell is a human cell.

In a further embodiment, the present disclosure comprises a method, wherein the cell is a human or mouse fibroblast, or a mammalian umbilical cord blood stem cell.

Another aspect of the present disclosure relates to an induced dendritic cell obtained by the method of the present disclosure.

Another aspect of the present disclosure relates to an induced dendritic cell transduced with the construct or vector as described herein, or the one more vectors as described herein.

In a further embodiment, the present disclosure relates to an induced dendritic cell obtainable by the method of the present disclosure, in a therapeutically effective amount and a pharmaceutically acceptable excipient.

In a further embodiment, the present disclosure comprises a method for use in veterinary or human medicine.

In a further embodiment, the present disclosure comprises a method for use in immunotherapy, or in the treatment or therapy of neurodegenerative diseases, or in autoimmune diseases, immunodeficiency, or in the treatment or therapy of cancer or in the treatment or therapy of an infectious diseases.

In an embodiment, the present disclosure comprises a method further comprising an anti-viral, an analgesic, an anti-inflammatory agent, a chemotherapy agent, a radiotherapy agent, an antibiotic, a diuretic, or mixtures thereof.

In a further embodiment, the present disclosure comprises a composition further comprising a filler, a binder, a disintegrant, or a lubricant, or mixtures thereof.

In a further embodiment, the present disclosure comprises a composition for use in intradermal and transdermal therapies.

In a further embodiment, the present disclosure comprises an injectable formulation, in particular an in-situ injection.

In an embodiment, the present disclosure comprises a composition for use in veterinary or human medicine, in particular in immunotherapy, or in neurodegenerative or ageing diseases, or in cancer or in infectious diseases, as a drug screening.

In a further embodiment, the present disclosure comprises a composition for use in the treatment, therapy or diagnosis of a central and peripheral nervous system disorder.

In a further embodiment, the present disclosure comprises a composition for use in the treatment, therapy or diagnosis of neoplasia in particular cancer, namely solid or hematological tumors.

In a further embodiment, the present disclosure comprises a composition for use in the treatment, diagnosis or therapy of cancer or immunological diseases, namely autoimmune diseases, hypersensitivities, or immunodeficiency.

In an embodiment, the present disclosure comprises a composition for use in the treatment, therapy or diagnosis of a fungal, viral, chlamydial, bacterial, nanobacterial or parasitic infectious disease.

In a further embodiment, the present disclosure comprises a composition for use in the treatment, therapy or diagnosis of HIV, infection with SARS coronavirus, Asian flu virus, herpes simplex, herpes zoster, hepatitis, or viral hepatitis.

In a further embodiment, the present disclosure comprises a vaccine for cancer comprising the composition as described in any one of the previous claims, or an induced dendritic cell of the present disclosure, or mixtures thereof.

In an aspect, the present disclosure relates to a vaccine or an injectable formulation, in particular an in-situ injection, for cancer comprising the composition as described herein, or the induced dendritic cell as described herein, or mixtures thereof.

In a further embodiment, the present disclosure comprises a kit comprising at least one of the following components:
an induced dendritic cell of the present disclosure;
a composition as described in the present disclosure;
a vector or a construct of the present disclosure;
or mixtures thereof.

Surprisingly, the induced DCs generated by reprograming as described in the present disclosure display an intrinsic surface marker phenotype of conventional dendritic cells type 2 (CD11b), as well as cytokine secretion and antigen presentation in MHC-II molecules.

The present disclosure relates to compositions comprising the combination of at least two isolated transcription factors encoded by a sequence 90% identical to a sequence from the group consisting of: PU.1 (SEQ. ID. 1, SEQ. ID. 2, SEQ. ID. 4, SEQ. ID. 5)), IRF4 (SEQ. ID. 7, SEQ. ID. 8, SEQ. ID. 10, SEQ. ID. 11), PRDM1 (SEQ. ID. 13, SEQ. ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. 25, SEQ. ID. 26, SEQ. ID. 28, SEQ. ID. 29), TGIF1 (SEQ. ID. 31, SEQ. ID.32, SEQ. ID. 34, SEQ. ID. 35), RBPJ (SEQ. ID. 43, SEQ. ID. 44, SEQ. ID. 46, SEQ. ID. 47), RELB (SEQ. ID. 37, SEQ. ID. 25 38, SEQ. ID. 40, SEQ. ID. 41), as reprogramming or inducing factors of a cell selected from the group consisting of: stem cell or a differentiated cell, or mixtures thereof.

Polypeptide variants or family members having the same or a similar activity as the reference polypeptides encoded by the sequences referenced (SEQ. ID. 1 to 36) can be used in the compositions, methods, and kits described herein. 30 Generally, variants of a particular polypeptide encoding a DC-inducing factor for use in the compositions, methods, and kits described herein will have at least about 90%, at least about 91%, at least about 92%, at least about 93%, at least about 94%, at least about 95%, at least about 96%, at least about 97%, at least about 98%, at least about 99% or more sequence identity to that particular reference polynucleotide or polypeptide as determined by sequence alignment algorithms and parameters described herein and known to those skilled in the art

Methods for sequence alignment for comparison include GAP, BESTFIT, BLAST, FASTA and TFASTA. GAP uses the algorithm of Needleman and Wunsch ((1970) J Mol Biol 48: 443-453) to find the global (over the whole the sequence) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimize alignment between conserved motifs, as would be apparent to a person skilled in the art. The sequence identity values, which are indicated in the present subject-matter as a percentage were determined over the entire amino acid sequence, using BLAST with default parameters.

In an embodiment, any of the DNA encoded sequences of the present disclosure can be altered, substituted, or modified to contain one or more, preferably 0, 1, 2, 3, 4, 5, 6 of different deoxyribonucleotide bases.

In an embodiment, the present disclosure validated that in Clec9a reporter mouse, the majority of cDC2 are labeled with tdTomato fluorescent protein making this model suitable for screening cDC2-inducing factors. PU.1 has been described to play a key role on DC development and IRF4 has been described to ensure cDC2 specification. Additionally, both PU.1 and IRF4 are highly expressed on cDC2 subsets. Therefore, the present disclosure combined PU.1 and IRF4 with additional 33 cDC2-inducing candidates and performed an additive screen in Clec9a reporter mouse embryonic fibroblasts (MEFs).

In an embodiment, PU.1 combined with IRF4 and PRDM1 is sufficient to induce Clec9a reporter activation and the surface expression of the cDC2 surface marker CD11b. Additionally, the expression of major histocompatibility complex (MHC) class II molecules, important for DC functionality, is induced by PU.1 combined with IRF4 and PRDM1.

A polycistronic construct encoding PU.1 followed by IRF4 and PRDM1 increases the efficiency of Clec9a reporter activation. The resulting generated tdTomato⁺ cells display the ability to secrete pro-inflammatory TNF-α upon TLR stimulation and to present antigens loaded on MHC-II to CD4⁺ T cells, inducing their proliferation and activation.

In an embodiment, PU.1 combined with IRF4 and IRF2 induces significant reporter activation. Moreover, PU.1 combined with IRF4 and IRF2 result in an increased tdT+CD11b+ double positive cell population.

In an embodiment, PU.1 combined with IRF4 and POU2F2 or PU.1 combined with IRF4 and TGIF1 results in an increased expression of the CD11b, a cDC2-specific surface marker.

In an embodiment, PU.1 combined with IRF4 and RBPJ or PU.1 combined with IRF4 and RELB results in an increased expression of the CD11b, a cDC2-specific surface marker.

In summary, we show that PU.1 and IRF4 when combined with PRDM1, IRF2, RBPJ, RELB, POU2F2 or TGIF1 induce cDC2 phenotypes in fibroblasts. These findings provide insights into cDC2 heterogeneity specification. Future generation of cDC2 by direct reprogramming opens possibilities for inducing immune-promoting and tolerogenic responses using autologous-engineered cells.

In an embodiment, the combination of isolated transcription factors may be:
PU.1 (SEQ. ID. 1-SEQ. ID. 6), IRF4 (SEQ. ID. 7-SEQ. ID. 12) and PRDM1 (SEQ. ID. 13-SEQ. ID. 18);
PU.1 (SEQ. ID. 1-SEQ. ID. 6), IRF4 (SEQ. ID. 7-SEQ. ID. 12) and IRF2 (SEQ. ID. 19-SEQ. ID. 24);
or PU.1 (SEQ. ID. 1-SEQ. ID. 6), IRF4 (SEQ. ID. 7-SEQ. ID. 12) and POU2F2 (SEQ. ID. 25-SEQ. ID. 30);
or PU.1 (SEQ. ID. 1-SEQ. ID. 6), IRF4 (SEQ. ID. 7-SEQ. ID. 12) and TGIF1 (SEQ. ID. 31-SEQ. ID.36);
or PU.1 (SEQ. ID. 1-SEQ. ID. 6), IRF4 (SEQ. ID. 7-SEQ. ID. 12) and RBPJ (SEQ. ID. 43-SEQ. ID. 48);

or PU.1 (SEQ. ID. 1-SEQ. ID. 6), IRF4 (SEQ. ID. 7-SEQ. ID. 12) and RELB (SEQ. ID. 37-SEQ. ID. 42).

Another aspect of the present disclosure is the use of a combination of at least two sequences from the group consisting of PU.1 (SEQ. ID. 1-SEQ. ID. 6), IRF4 (SEQ. ID. 7-SEQ. ID. 12), PRDM1 (SEQ. ID. 13-SEQ. ID. 18), IRF2 (SEQ. ID. 19-SEQ. ID. 24), POU2F2 (SEQ. ID. 25-SEQ. ID. 30) and TGIF1 (SEQ. ID. 31-SEQ. ID.36). The isolated transcription factors may include the following combination:
PU.1 (SEQ. ID. 1-SEQ. ID. 6) and IRF4 (SEQ. ID. 7-SEQ. ID. 12); or PU.1 (SEQ. ID. 1-SEQ. ID.6) and PRDM1 (SEQ. ID. 13-SEQ. ID. 18); or
IRF4 (SEQ. ID. 7-SEQ. ID. 12) and PRDM1 (SEQ. ID. 13-SEQ. ID. 18); or
PU.1 (SEQ. ID. 1-SEQ. ID.6) and IRF2 (SEQ. ID. 19-SEQ. ID. 24); or
PU.1 (SEQ. ID. 1-SEQ. ID.6) and POU2F2 (SEQ. ID. 25-SEQ. ID. 30);
or PU.1 (SEQ. ID. 1-SEQ. ID.6) and TGIF1 (SEQ. ID. 31-SEQ. ID. 36);
or IRF4 (SEQ. ID. 7-SEQ. ID. 12) and IRF2 (SEQ. ID. 19-SEQ. ID. 24);
or IRF4 (SEQ. ID. 7-SEQ. ID. 12) and POU2F2 (SEQ. ID. 25-SEQ. ID. 30);
or IRF4 (SEQ. ID. 7-SEQ. ID. 12) and TGIF1 (SEQ. ID. 31-SEQ. ID. 36);
or PRDM1 (SEQ. ID. 13-SEQ. ID. 18) and IRF2 (SEQ. ID. 19-SEQ. ID. 24);
or PRDM1 (SEQ. ID. 13-SEQ. ID. 18) and POU2F2 (SEQ. ID. 25-SEQ. ID. 30);
or PRDM1 (SEQ. ID. 13-SEQ. ID. 18) and TGIF1 (SEQ. ID. 31-SEQ. ID. 36);
or PU.1 (SEQ. ID. 1-SEQ. ID.6) and IRF2 (SEQ. ID. 19-SEQ. ID. 24);
or IRF2 (SEQ. ID. 19-SEQ. ID. 24) and POU2F2 (SEQ. ID. 25-SEQ. ID. 30);
or IRF2 (SEQ. ID. 19-SEQ. ID. 24) and TGIF1 (SEQ. ID. 31-SEQ. ID. 36);
or PU.1 (SEQ. ID. 1-SEQ. ID.6) and POU2F2 (SEQ. ID. 25-SEQ. ID. 30);
or POU2F2 (SEQ. ID. 25-SEQ. ID. 30) and TGIF1 (SEQ. ID. 31-SEQ. ID. 36);
or PU.1 (SEQ. ID. 1-SEQ. ID.6) and TGIF1 (SEQ. ID. 31-SEQ. ID. 36).

In an embodiment for better results the cell may be selected from the group consisting of: pluripotent stem cell, multipotent stem cell, differentiated cell, tumor cell, cancer cell, and mixtures thereof. In particular a mammalian cell, more in particular a mouse or a human cell.

In an embodiment, the isolated transcription factor of the present disclosure may be used for veterinary or human medicine applications, in particular in infectious disease, or viral disease, or viral induced disease, or neurodegenerative diseases, or in cancer, or in diabetes, or in immunotherapy, or in autoimmune disease, or in hypersensitivity disease.

In an embodiment for better results, the isolated transcription factor of the present disclosure may be used as a reprogramming or inducing factor of a cell selected from the group consisting of: pluripotent stem cell, or multipotent stem cell, or differentiated cell, and mixtures thereof into a dendritic cell or interferon-producing cell.

### DESCRIPTION OF DRAWINGS

The following figures provide preferred embodiments for illustrating the disclosure and should not be seen as limiting the scope of invention.

FIG. 1—Ontogeny of the 3 main DC subsets. DCs emerge from a common DC precursor (CDPs) in the bone marrow which can develop into different DC subsets: cDC1, which mainly perform antigen cross presentation promoting Th1 responses and cytotoxic T-cell responses; pDC which act as interferon type I-producing cells upon viral infection; cDC2, a DC subset mainly performing MHC-II antigen presentation and promoting Th2, Th17 and Treg responses.

FIG. 2—Schematic representation of the applications of direct reprogrammed cDC2. Fibroblasts obtained from patients will be reprogrammed into cDC2 cells that can be applied for personalized immunotherapy. Induced cDC2s can be used to induce immunity against parasites, against extracellular pathogens, to promote anti-tumor responses or to induce immune tolerance to self-antigens in the context of autoimmunity or hypersensitivity.

FIG. 3—Splenic cDC2 express high levels of tdTomato protein driven by the Clec9a-tdTomato reporter. (A) Flow cytometry analysis of tdTomato expression in splenic cDC1 (MHC-II⁺ CD11c⁺ CD8a⁺) and cDC2 (MHC-II⁺ CD11c⁺ CD11b⁺) populations isolated from Clec9a-tdTomato mice. (B) Quantification of tdTomato⁺ cells in cDC1 (CD8α⁺) and cDC2 (CD8α⁻CD11b⁺).

FIG. 4—Clec9a-reporter activation and gene expression patterns are surprisingly suitable to identify factors for cDC2 instruction. (A) Experimental strategy to screen for cDC2-inducing transcription factors (TFs). PU.1, IRF8 and BATF3 (PIB) combination induces reprogramming of Clec9a-tdTomato (Clec9a-tdT) mouse embryonic fibroblasts (MEFs) into cDC1-like induced cells. This combination will be modified to keep the fundamental TFs for cDC and identify combinations for cDC2 reprogramming. (B) Comparison of the expression of Spit Irf8 and Batf3 in cDC1 and cDC2 (GSE15907). Fold change in gene expression is depicted in brackets. (C and D) Quantification of tdTomato⁺ cells at day 6 after transduction with combinations of PU.1+BATF3 and the different members of the IRF family (IRF1-IRF9), or PU.1+IRF4. (E) Expression of the Irf gene family in cDC1 and cDC2 (GSE15907). Fold change is depicted in brackets. *P<0.05, **P<0.01, ***P<0.001, ****P<0.00001, unpaired t-test and one-way ANOVA.

FIG. 5—Strategy to identify cDC2-inducing transcription factors. (A) Schematic representation of the screening strategy for cDC2 reprogramming combinations. PU.1 and IRF4 were overexpressed along with additional individual candidate TFs. Clec9a reporter activation and expression of the cDC2 surface marker CD11b was assessed at day 6 of reprogramming (B) Candidate TFs are highly enriched in cDC2 when compared with cDC1 and pDC populations. Heatmap of the expression of PU.1, IRF4 and the 33 candidates in cDC1, cDC2 and pDC populations (GSE15907).

FIG. 6—Clec9a reporter based screening for cDC2-inducing TFs identifies new regulators of cDC2 reprogramming. Flow cytometry analysis representative plots (A) and quantification of tdTomato positive (tdTomato^{—}) (B) cells at day 6 after transduction of Clec9a reporter MEFs with PU.1+IRF4 in combination with the individual additional candidates (mean±SD; screening data and statistics of 2 to 7 replicates per condition). MEFs transduced with M2rtTA, PU.1+IRF8+BATF3 and PU.1+IRF4+BATF3 were included as controls. *P<0.05, **P<0.01, one-way ANOVA.

FIG. 7—CD11b expression-based screening for cDC2-inducing TFs identifies new regulators of cDC2 reprogramming. Flow cytometry analysis representative plots (A) and quantification of CD11b+ (B) cells at day 6 after transduction of Clec9a reporter MEFs with PU.1+IRF4 in combination with the individual additional candidates (mean±SD; screening data and statistics of 2 to 7 replicates per condition). MEFs transduced with M2rtTA, PU.1+IRF8+BATF3 and PU.1+IRF4+BATF3 were included as controls. *P<0.05, **P<0.01, one-way ANOVA.

FIG. 8—Induction of cDC2-like cells from mouse fibroblasts with combinations of three transcription factors. Flow cytometry analysis representative plots (A) and quantification of double positive tdTomato⁺CD11b⁺ (B) cells at day 6 after transduction with PU.1+IRF4 in combination with the individual additional candidates (mean±SD, screening data consisting of 2 replicates per condition). M2rtTA-transduced MEFs were included as controls.

FIG. 9—PU.1, IRF4 and PRDM1 are sufficient and required for cDC2 reprogramming. Quantification of tdT⁺ cells (A) and CD11b⁺ cells gated within the tdT⁺ population (B) after transduction of PU.1+IRF4+PRDM1 and individual removal of TFs from the 3 TF pool or individual TF expression at day 6 (n=2, mean 35 SD). M2rtTA-transduced MEFs were included as controls.

FIG. 10—PU.1, IRF4 and PRDM1 are enriched in cDC2 cells. (A) Gene expression of Spi1, Irf4 and Prdm1 in DC populations (pDC, cDC1 and cDC2). Spi1, Irf4 and Prdm1 are more expressed in cDC2 and Prdm1 is specifically more expressed in cDC2. (B) The combination of Spi1, Irf4 and Prdm1 is mostly enriched in CD8a- DCs among 96 mouse tissues and cell-types. Gene expression data (GeneAtlas MOE430) log transformed and normalized to a 0-1 range for each gene with a followed by a search for highest average expression for Spi1+Irf4+Prdm1.

FIG. 11—PU.1, IRF4 and PRDM1 induce CD45 and MHC-II surface expression. (A) Representative flow cytometry plots and (B) quantification of MHC-11⁺ cells in M2rtTA, PU.1+IRF8+BATF3 and PU.1+IRF4+PRDM1 transduced MEFs at day 6 (n=2, mean±SD). (C) Quantification of MHC-II⁺ cells within tdT⁺ in M2rtTA, PU.1+IRF8+BATF3 and PU.1+IRF4+PRDM1 transduced MEFs at day 6 (n=2, mean±SD). (D) CD45 and MHC-II expression within tdT⁻ and tdT⁺ populations in PU.1+IRF4+PRDM1 transduced MEFs at day 9.

FIG. 12—Combination of PU.1 and IRF4 is sufficient for Clec9a reporter activation. Quantification of tdTomato positive (tdTomato⁺) cells at day 6 after transduction of Clec9a reporter MEFs with PU.1 in combination with the individual additional candidates (mean±SD; screening data of 2 replicates per condition). M2rtTA-transduced MEFs were included as controls.

FIG. 13—Combinations of DC2-inducing TFs induce gradual Clec9a reporter activation. Kinetics of Clec9a-tdTomato reporter activation for PU.1+IRF4+PRDM1 (P+I4+P) and PU.1+IRF4+IRF2 (P+I4+I2) combinations. M2rtTA-transduced MEFs were included as control.

FIG. 14—PU.1, IRF4 and IRF2 is a minimal and sufficient combination to induce a DC phenotype independent of PRDM1. (A) Quantification of tdTomato⁺ cells after transduction with PU.1+IRF4+IRF2 and individual removal of TFs from the 3 TF pool or individual TF expression at day 6 (n=2, mean±SD). (B) The combination of Spi1, Irf4 and Irf2 is mostly enriched in CD8a- DCs among 96 mouse tissues and cell-types. Gene expression data (GeneAtlas MOE430) log transformed and normalized to a 0-1 range for each gene with a followed by a search for highest average expression for Spi1+Irf4+Irf2. (C) Quantification of TdTomato⁺ cells after transduction with PU.1+IRF4 combined with PRDM1, IRF2 or PRDM1+IRF2 at day 6 of reprogramming. (D) Schematic representation of the polycistronic construct encoding Spi1 followed by Irf4 and Irf2, separated by self-cleaving peptides P2A and T2A in the pFUW-TetO plasmid (PI₄I_{Poly}). (E) Quantification of TdTomato⁺ cells after transduction with M2rtTA, the individual PU.1, IRF4 and IRF2 factors (P+I₄I₂) and PI₄I_{Poly} construct at day 6 of reprogramming.

FIG. 15—Polycistronic PLR vector (PI₄P_{Poly}) increases reprogramming efficiency. (A) Schematic representation of the polycistronic construct encoding Spi1 followed by Irf4 and Prdm1, separated by self-cleaving peptides P2A and T2A inserted in the pFUW-TetO plasmid (PI₄P_{Poly}). (B) Representative flow cytometry plots and (C) quantification of TdT⁺ cells after transduction with M2rtTA, PU.1, IRF4 and PRDM1 encoded by individual vectors (P+I₄+P), polycistronic PU.1, IRF8 and BATF3 combination (PI₈B_{Poly}) and polycistronic PU.1, IRF4 and PRDM1 (PI₄P_{Poly}) constructs at day 6 of reprogramming (mean±SD, n=2). (D) Fluorescence microscopy comparison of M2rtTA, PI₈B_{Poly} and PI₄P_{Poly}-transduced MEFs at day 9 of reprogramming representing tdT⁺ cell morphology (white arrows).

FIG. 16—PIP-induced cells secrete TNF-α pro-inflammatory cytokine. Quantification of TNF-α and IL-10 concentrations in the supernatants of FACS-sorted tdTomato+ cells at day 9 of reprogramming induced by PU.1, IRF4 and PRDM1 polycistronic vector (PI₄P_{Poly}) before (−) of after overnight TLR stimulation with LPS, polyl:C (PiC), R848 and CpG ODN 1585.

FIG. 17—PIP expression induces ability to present antigens in MHC-II molecules to CD4⁺ T-cells in an antigen-specific manner. Quantification of CTV dilution of OVA-specific OT-II Rag2KO CD4⁺ cells (CD4⁺ TCRb⁺) after coculture of MEFs, sorted PIP-TdT⁺ cells (day 9) and bone-marrow DCs (BM-DC) previously loaded with or without OVA peptide (323-339) and upon different stimulation conditions: No stimulation (−), LPS, PiC, R848 and CpG ODN 1585.

### DETAILED DESCRIPTION

The present disclosure relates to compositions, nucleic acid constructs, methods and kits thereof for reprogramming cells into conventional dendritic cells, particularly into conventional dendritic cells type 2 (cDC2), methods and uses thereof, particularly to the development of methods for making cDCs, particularly into cDC2, from differentiated, multipotent or pluripotent stem cells by introducing and expressing isolated/synthetic transcription factors. More particularly, the disclosure provides methods for obtaining cDCs, particularly cDC2, by direct cellular reprogramming with the surprisingly beneficial use of combinations of specific transcription factors. Such compositions, nucleic acid constructs, methods and kits can be used for inducing dendritic cells in vitro, ex vivo, or in vivo, and these induced DCs or APCs can be used for immunotherapy applications.

Natural DCs are bone marrow-derived cells that are seeded in all tissues. DCs are poised to sample the environment and to transmit the gathered information to cells of the adaptive immune system (T cells and B cells). Upon antigen engulfment, DCs initiate an immune response by presenting the processed antigen, which is in the form of peptide-major histocompatibility complex (MHC) molecule complexes, to naïve (that is, antigen inexperienced) T cells in lymphoid tissues. After activation, DCs typically overexpress co-stimulatory and MHC molecules in addition to secrete various cytokines responsible for initiating and/or enhancing many T and B lymphocyte responses, i.e. type I interferon, tumor necrosis factor (TNF)-α, IFN-γ, IL-12 and IL-6.

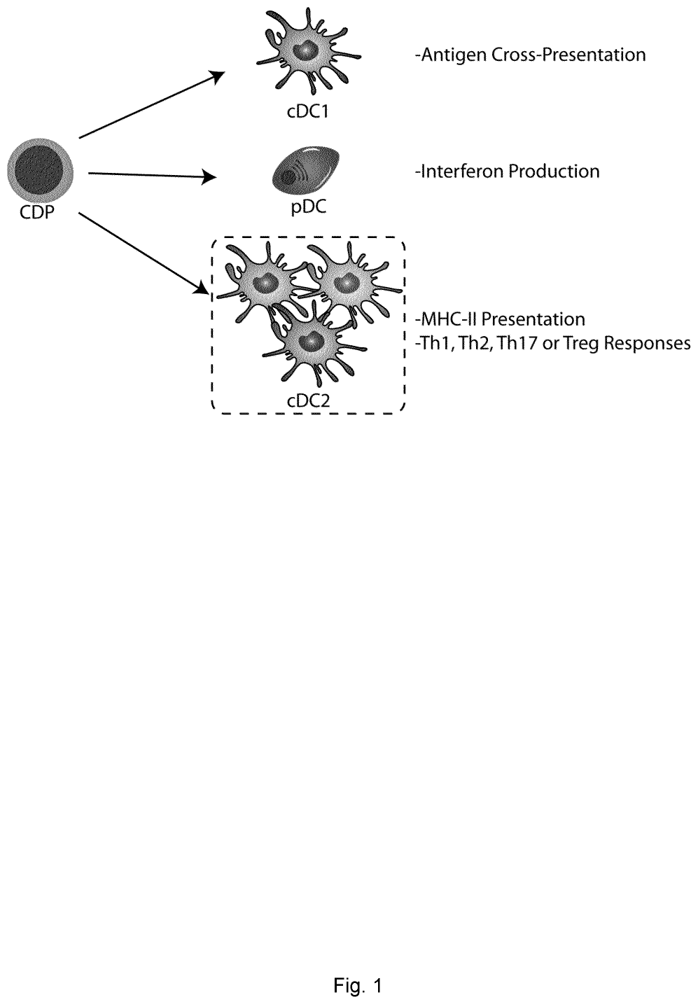

Thus, DCs are generally identified by their high expression of major histocompatibility complex class II molecules (MHC-II), co-stimulatory molecules, such as CD80/86 and CD40, and integrin CD11c, as well as their superior capacity to secrete inflammatory cytokines and to migrate from non-lymphoid to lymphoid organs and stimulate naïve T cells. In mice and humans, distinct subsets of DCs can be variably defined by phenotype, ontogeny, and function (FIG. 1). They include the conventional DC subset 1 (cDC1, also known as CD8α⁺ DC subset) found in the lymphoid organs that display the ability of cross-presentation on MHC class I and trigger CTL responses against infectious agents or tumors. pDCs act by producing high amounts of type I interferon in response to viral infections. cDC2, on the other hand, excel in MHC-II presentation leading towards Th2 and Th17 T cell responses. In addition to priming T cells, cDC2 have been implicated in the establishment of self-tolerance to antigens by priming Tregs or by contributing to the negative selection of autoreactive T cells in the thymus. DNGR-1, also known as CLEC9A, is a receptor for necrotic cells that favors cross-priming of CTLs to dead cell-associated antigens in mice. DNGR-1 is selectively expressed at high levels by mouse cDC1 DCs, cDC2 DCs and pDCs. Recently, expression of Clec9a was shown to allow the identification of DC precursors (CDPs) committed to the conventional or plasmacytoid DC lineage and lineages and their progeny in lymphoid tissues.

The successful identification of DC-inducing factors capable of reprogramming differentiated cells to induced DCs, specifically cDC2, as described herein, can advance our basic understanding of cDC2 biology and heterogeneity in a number of ways. This work will provide thorough insight into cDC2 transcriptional networks. In addition, the identification of DC-inducing factors offers unprecedented opportunities to understand how the DC state is established and how key regulatory machinery is put into place.

Transcription factors play a critical role in the specification of all cell types during development. The success of direct reprogramming strategies using transcription factor-mediated reprogramming indicates that it is equally plausible to direct the differentiation of pluripotent ES/iPS cells or multipotent stem cells to specific fates using such factors. Accordingly, using the DC-inducing factors identified herein, directed differentiation of ES/iPS cells to a definitive DC fate by expression of the DC-enriched transcription factors can be achieved. Additionally, using the DC-inducing factors identified herein, directed differentiation of multipotent hematopoietic stem and progenitor cells to a definitive DC fate by expression of the DC-enriched transcription factors can be achieved.

An aspect of the present disclosure is the use of TFs or the use of a combination of TFs to generate cells that can present self-antigens to generate tolerance responses. This method represents a feasible strategy for tolerogenic immunotherapies in context of autoimmune and hypersensitivity disorders

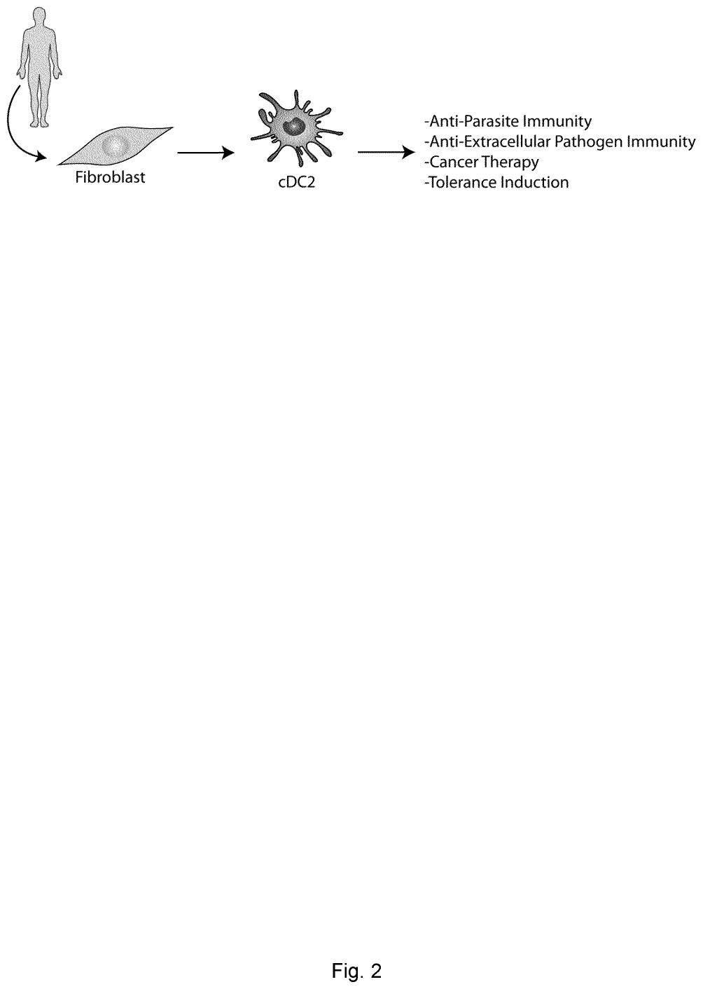

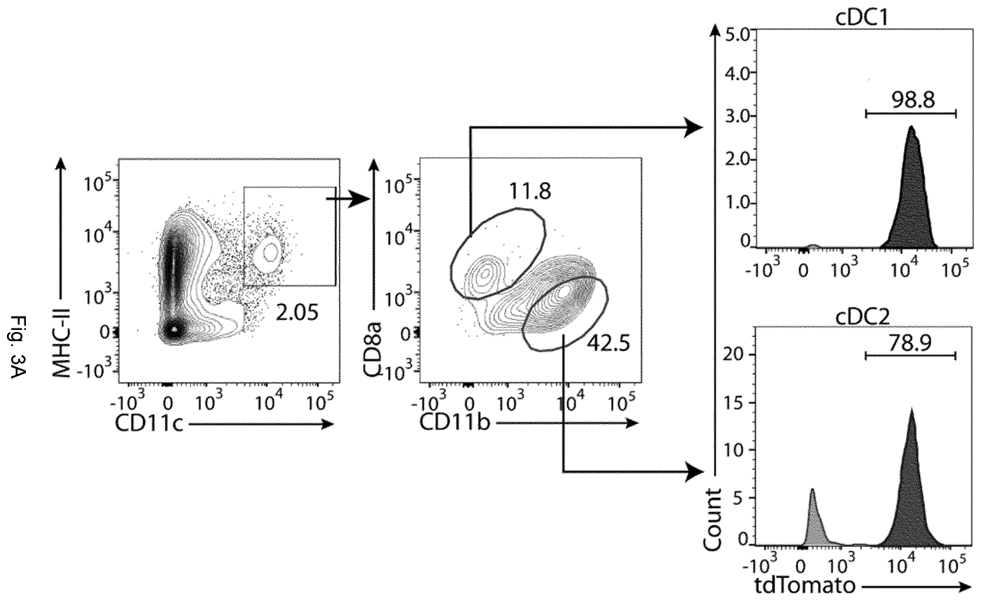

Fibroblasts can be obtained from a human source and then reprogrammed to cDC2 for immune modulating purposes (FIG. 2). According to the known immune roles of cDC2, these generated cDC2s can be applied to promote anti-parasite immunity, immunity against extracellular pathogens, immune tolerance to self-antigens in the context of autoimmunity or hypersensitivity or even to promote anti-tumor immunity when combined with cDC1.

Nucleic acids encoding the DC-inducing factors, e.g., DNA or RNA, or constructs thereof, are introduced into a cell, using viral vectors or without viral vectors, via one or repeated transfections, and the expression of the gene products and/or translation of the RNA molecules result in cells that are morphologically, biochemically, and functionally similar to cDC2, as described herein. These induced cDC2s express the cDC2 surface marker CD11b.

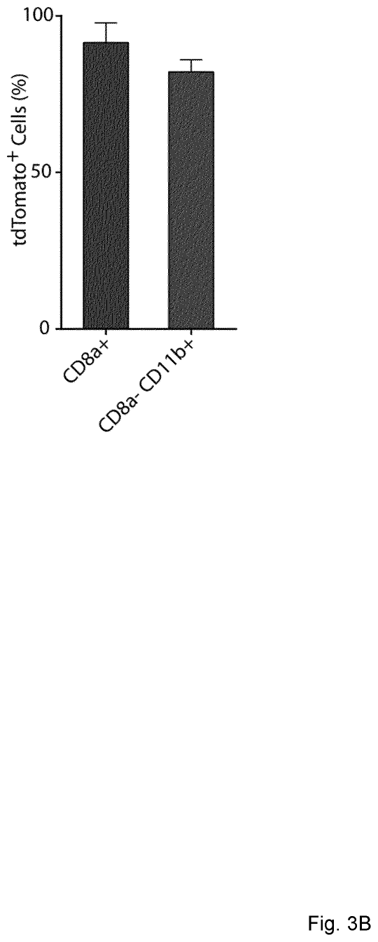

In an embodiment, in order to screen the effect of the cDC2-inducing TFs and cDC2-inducing TF combinations by cellular reprogramming, Mouse Embryonic Fibroblasts (MEFs) harboring a DC-specific reporter (Clec9a-Cre X R26-stop-tdTomato) were used, where the activation of the reporter was used to show cDC2-inducing TFs. In Clec9a-tomato reporter mice, the tdTomato fluorescent protein is expressed exclusively by CDPs, pre-DCs, cDCs and pDCs. Macrophages, other immune lineages or monocyte-derived DCs in culture do not express Clec9a and therefore neither the tdTomato protein. Spleen cells isolated from Clec9a reporter mice were analyzed, confirming that 78.9% of cDC2 cells (gated in CD11c⁺MHC-II⁺CD8a⁻ CD11b⁺) express the tdTomato fluorescent protein (FIG. 3).

Double transgenic Clec9a-tdTomato reporter MEFs were isolated from E13.5 embryos and excluded from any contaminating tdTomato⁺ or CD45⁺ cell that could already have been committed to the hematopoietic lineage, by the use of Fluorescent-Activated Cell Sorting (FACS).

Reprogramming of fibroblasts to cDC1-like cells was recently demonstrated through the combined overexpression of PU.1, IRF8 and BATF3 (PIB).

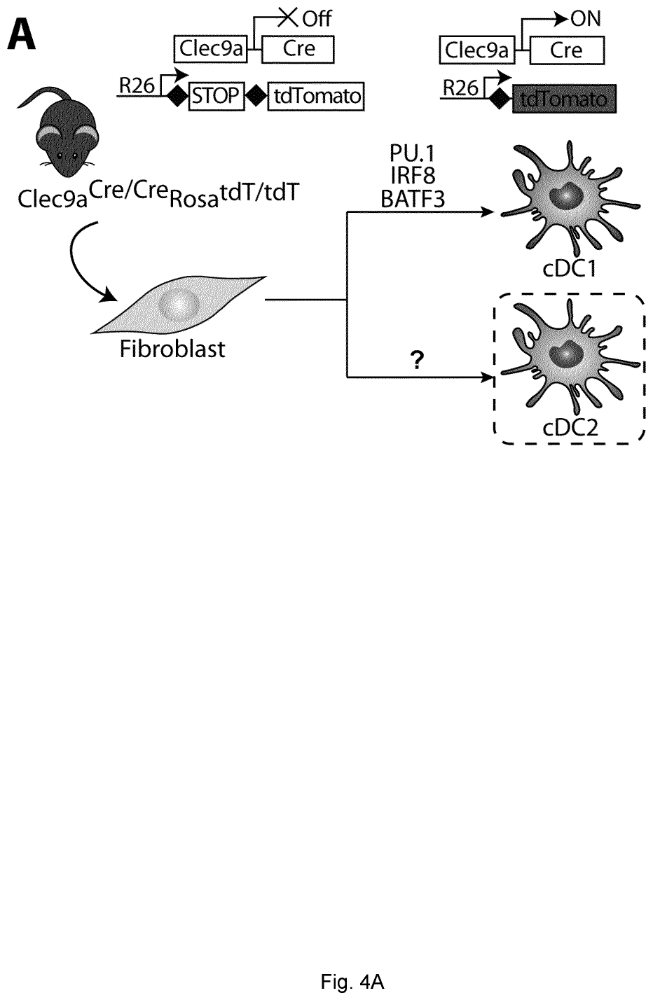

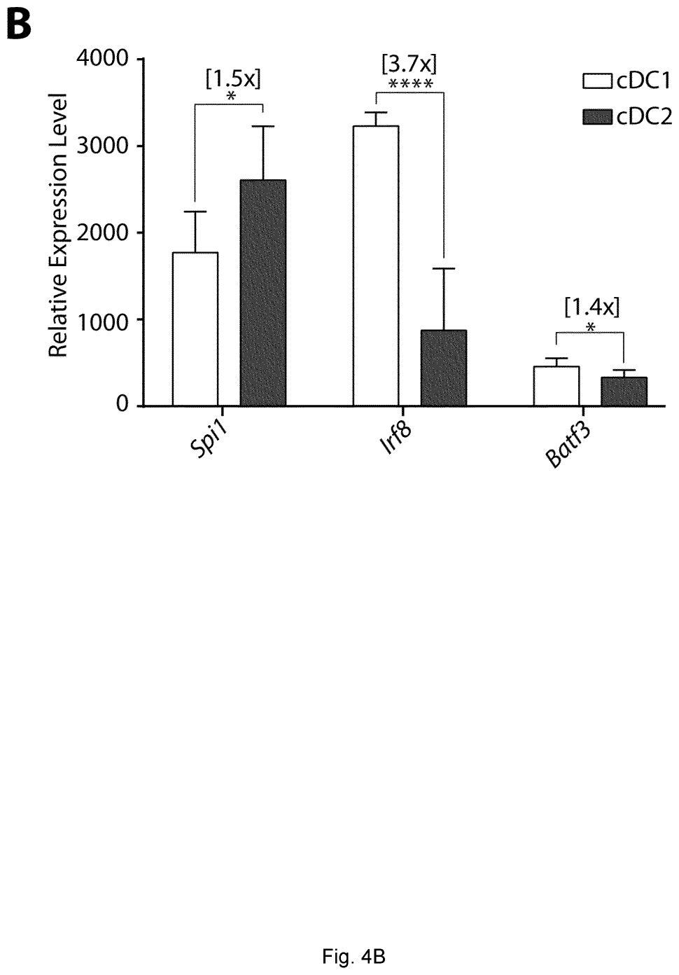

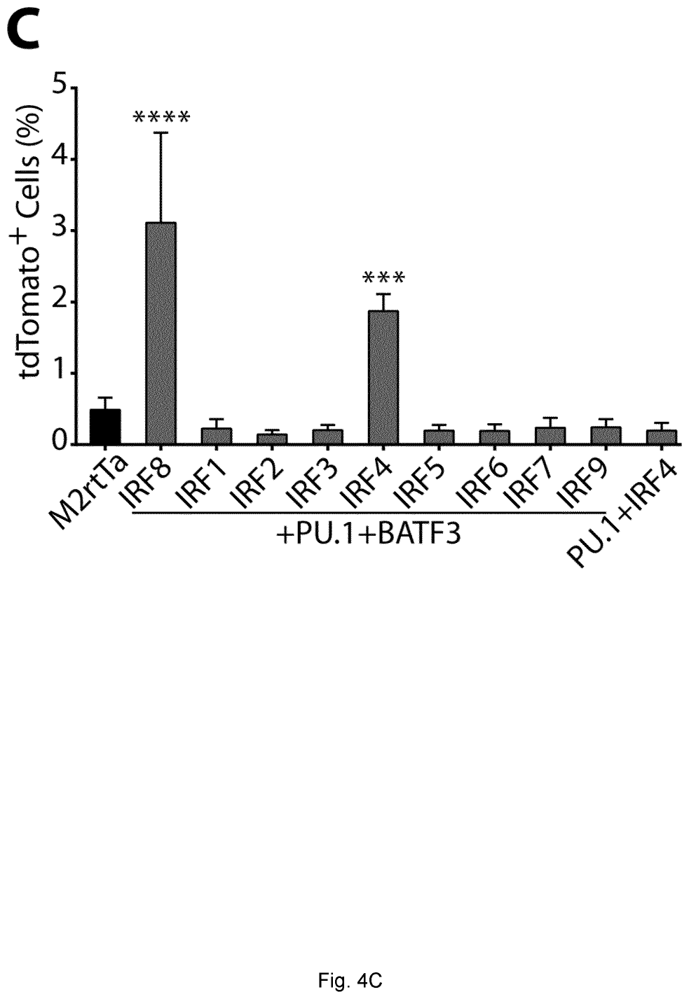

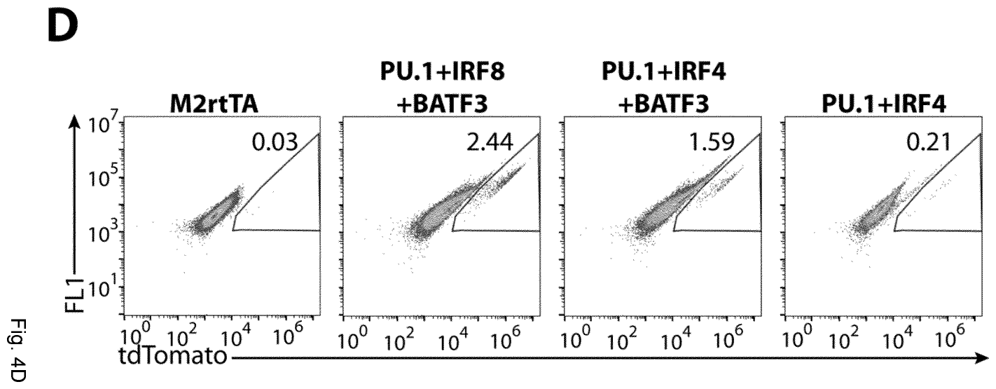

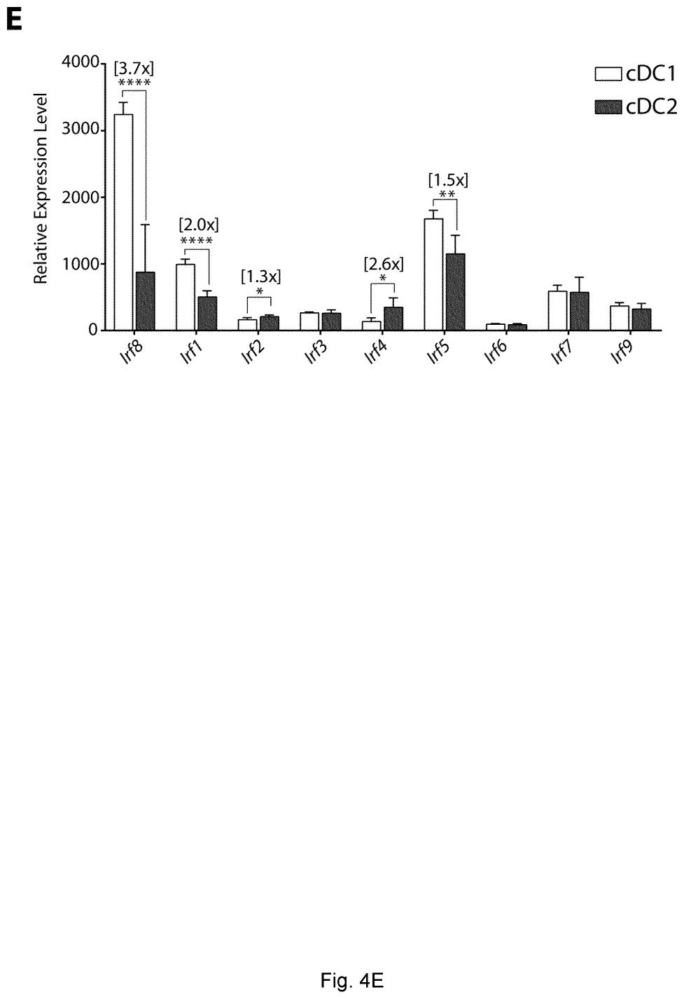

This combination was identified by screening employing the Clec9a-Cre X R26-stop-tdT (Clec9a-tdT) mouse that is expressed in cDC1s, cDC2s and pDCs (Rosa et al., 2018) . Here, this same DC reporter was used to identify cDC2-transcription factors that instruct cDC2 lineage by modifying the reported combination of TFs (FIG. 4A).

By comparing the expression of Spi1, Irf8 and Batf3 between cDC populations, Spi1 was shown to be highly expressed in cDC2 while Irf8 and Batf3 were less expressed when compared with cDC1s (FIG. 4B). Moreover, according to loss-of-function studies, PU.1 loss impairs specification of the whole DC lineage suggesting its continuous requirement for DC development. Together, these data support PU.1 maintenance for cDC2 reprogramming and suggest that IRF8 and BATF3 could be substituted by other TFs.

Since the IRF family of TFs is known to be crucial for DC development, maturation and functional roles (Gabriele & Ozato, 2007), the substitution of IRF8 by other IRF proteins in combination with PU.1 and BATF3 was tested for activation of the Clec9a reporter. IRF4 led to significant tdT expression (FIG. 4C), suggesting that IRF4 is able to replace IRF8 in DC reprogramming. IRF4 is believed to be necessary for cDC2 development. IRF8 and BATF3, on the other hand, are only believed to be critical for cDC1 development, suggesting their negligibility for cDC2 reprogramming. However, combined overexpression of PU.1 and IRF4 did not result in a significant Clec9a reporter activation (0.21%, FIGS. 4C and D) suggesting that these two TFs may be required but not sufficient to induce cDC2 reprogramming. Also, when comparing the expression of all Irf gene family members between cDC1 and cDC2 populations, Irf4 is significantly more expressed in cDC2s (FIG. 4E), further evidencing its importance for cDC2 reprogramming. Indeed, from the whole IRF family only Irf4 (2.6-fold) and to a lower extend Irf2 (1.3-fold) are overrepresented in cDC2 cells.

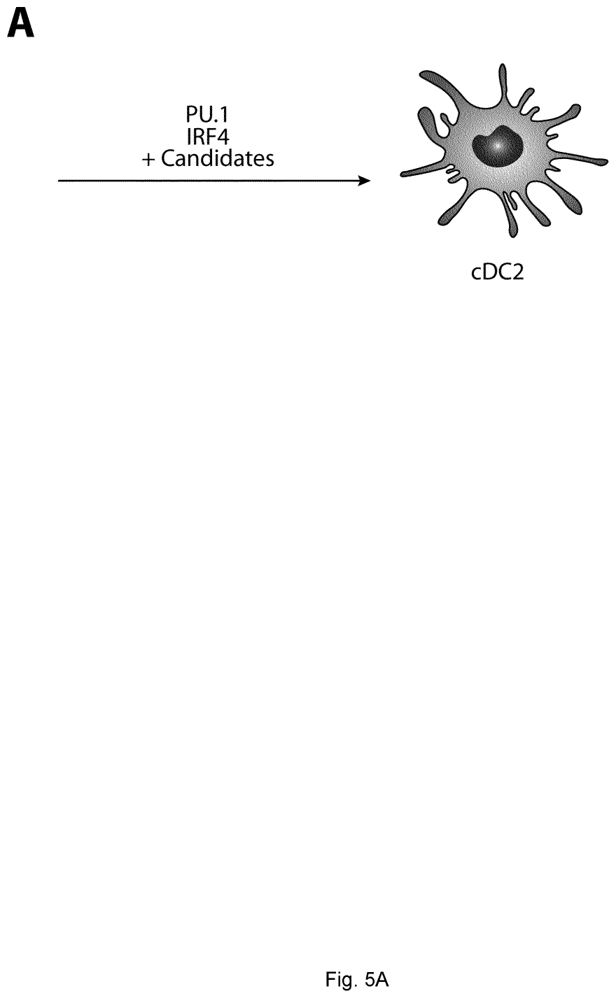

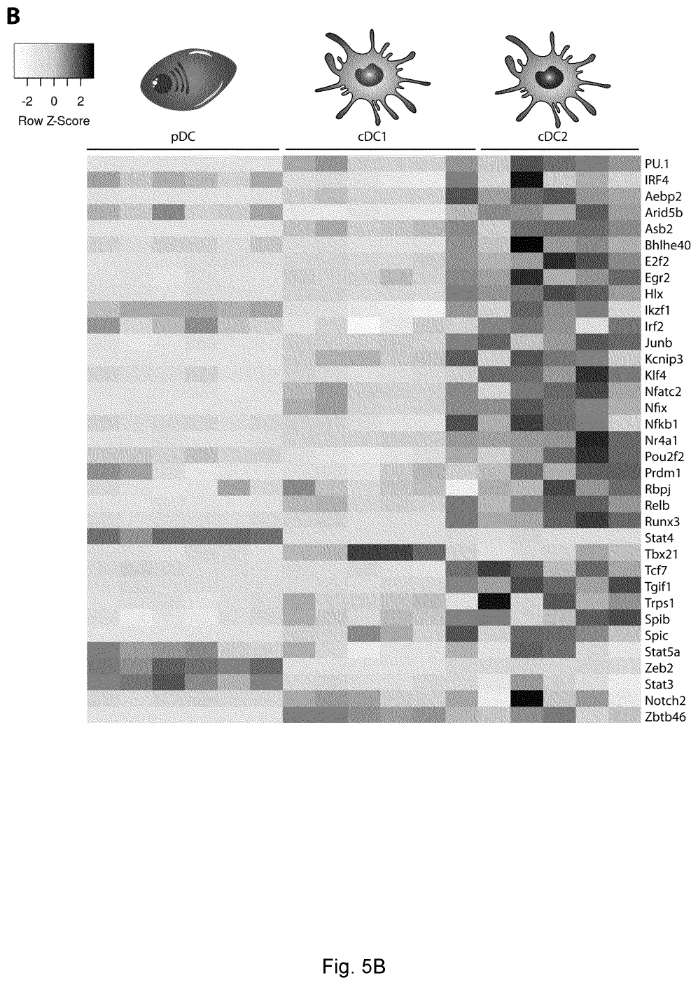

In an embodiment, to screen for cDC2 reprogramming TF combinations, the candidate TFs were individually combined with PU.1 and IRF4 and assessed for Clec9a reporter activation and expression of the cDC2 surface marker CD11b (FIG. 5A).

In an embodiment, 33 cDC2-inducing candidate TFs were selected due to their specifically enriched gene expression in cDC2s when compared to cDC1s and pDCs (FIG. 5B). These 33 candidate TFs, along with PU.1 and IRF4, were cloned individually in a reprogramming proven Doxycycline (Dox)-inducible lentiviral vector.

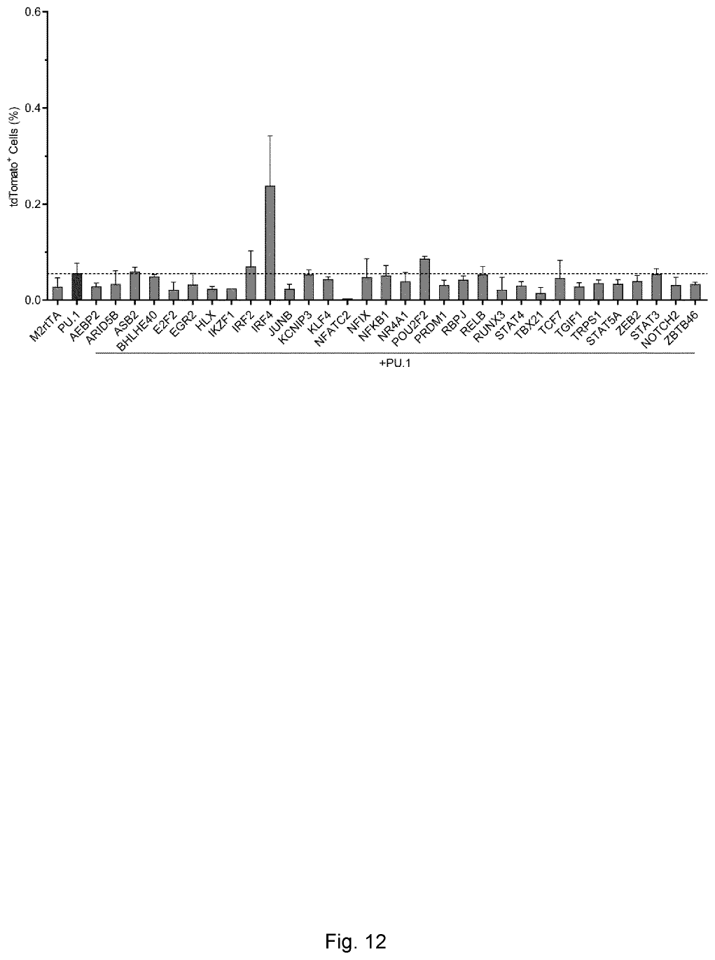

In an embodiment, to screen for cDC2 reprogramming TF combinations, the candidate TFs were first individually combined with PU.1 and assessed for Clec9a reporter activation (FIG. 12). From this screening, only the PU.1+IRF4 combination resulted in a noteworthy percentage of TdT⁺ cells, solidifying this combination as a baseline for further cDC2-inducing combinations.

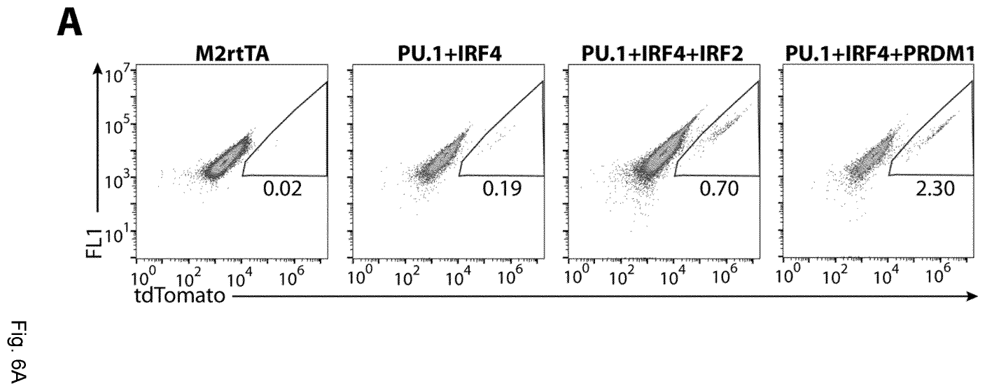

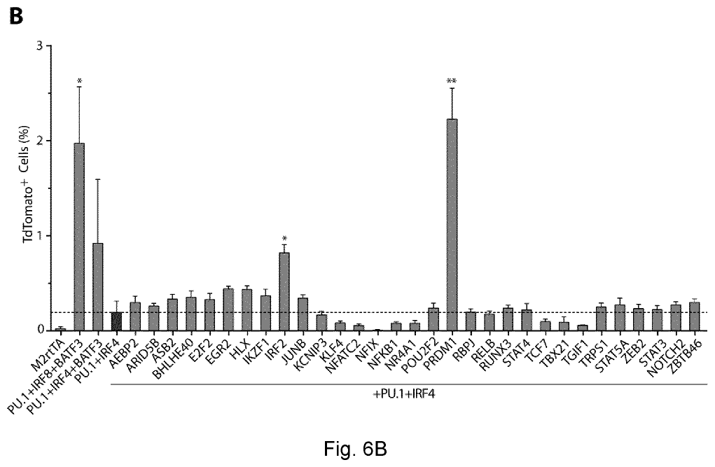

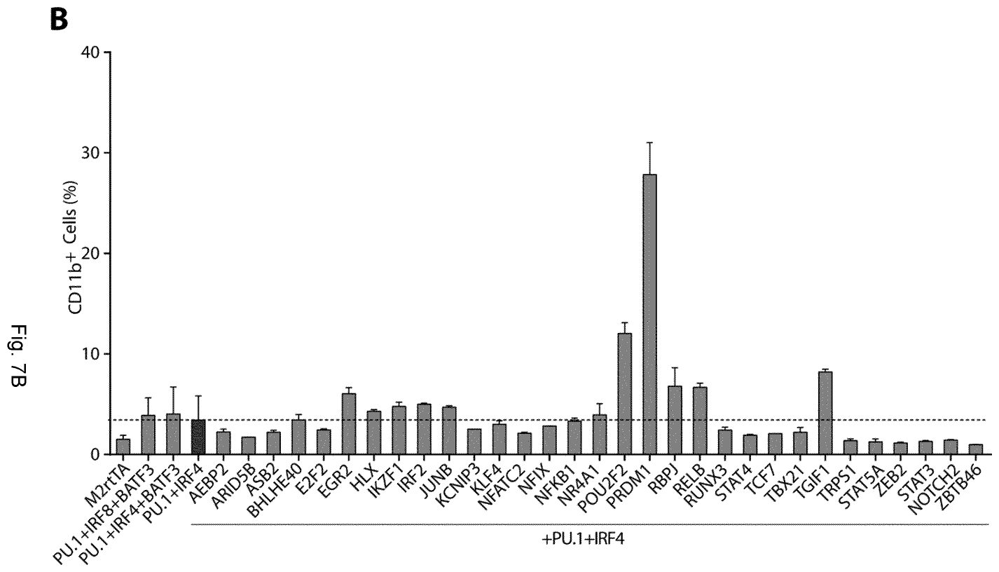

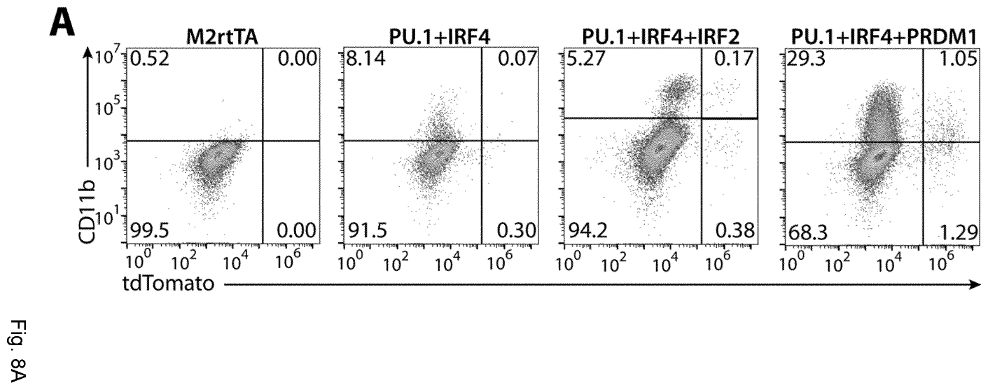

In an embodiment, screening of candidate TFs identified that PRDM1 or IRF2 in combination with PU.1 and IRF4 result in significant Clec9a reporter activation (FIG. 6) and an increased tdT⁺CD11b⁺ double population (FIG. 8). PRDM1 in combination with PU.1 and IRF4 also resulted in an increased expression of the cDC2 surface marker CD11b.

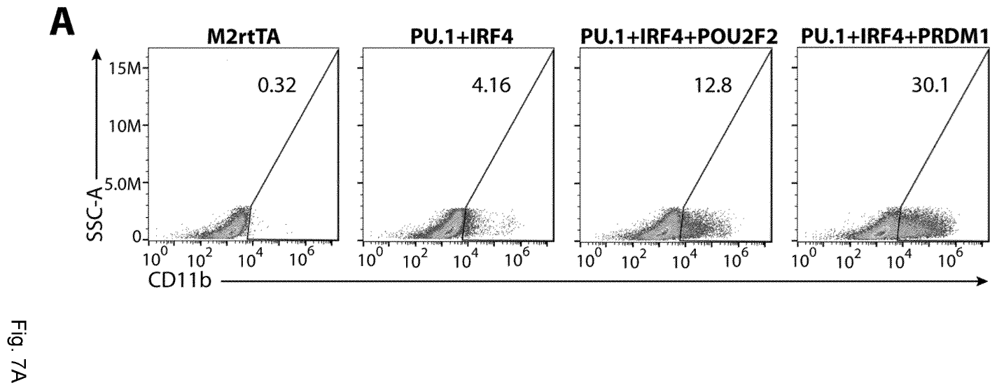

In an embodiment, PRDM1, RBPJ, RELB, POU2F2 or TGIF1 in combination with PU.1 and IRF4 result in increased expression of the cDC2 surface marker CD11b (FIG. 7). Collectively, these data identify PRDM1, RBPJ, RELB, POU2F2 and TGIF1 as additional cDC2-instructing TFs, possibly indicative of the induction of different cDC2 cell states, reflecting the inherent diversity within the cDC2 subset.

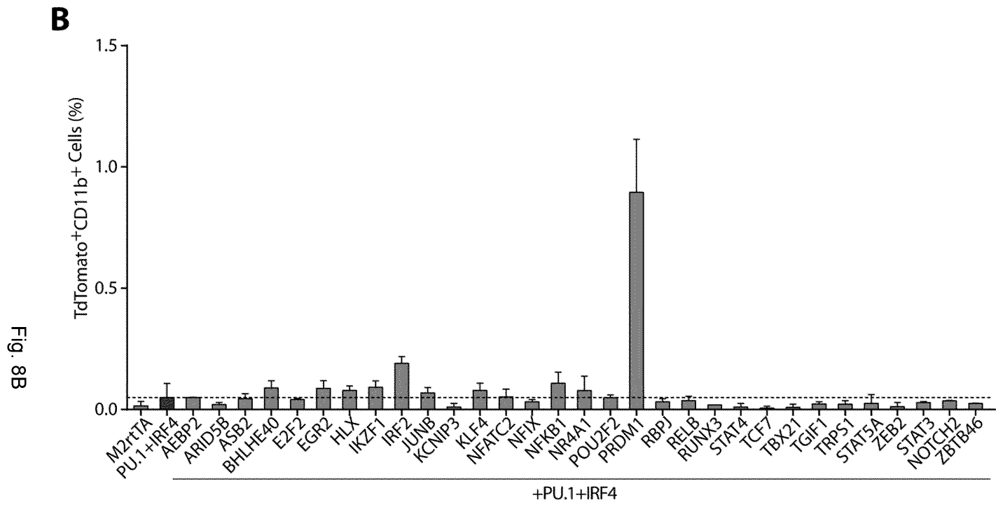

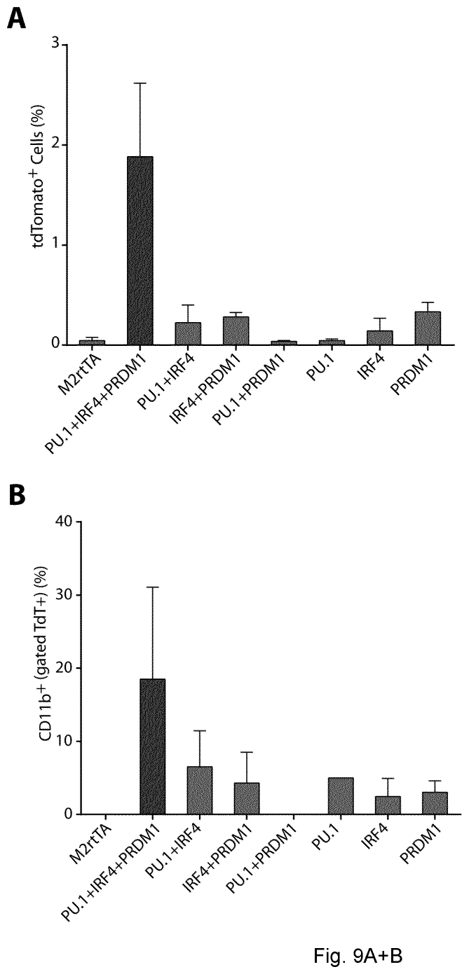

In an embodiment, to evaluate whether PU.1+IRF4+PRDM1 represent a minimal network for reprogramming, each of the factors were individually removed from the three TF pool. Removal of each of these individual TFs diminished Clec9a reporter activation and CD11b expression and individual expression of each TF generated low tdT and CD11b expression (FIGS. 9A and B). Collectively, this data implicates PU.1+IRF4+PRDM1 as a TF combination sufficient for Clec9a-tdT reporter activation and CD11b surface expression.

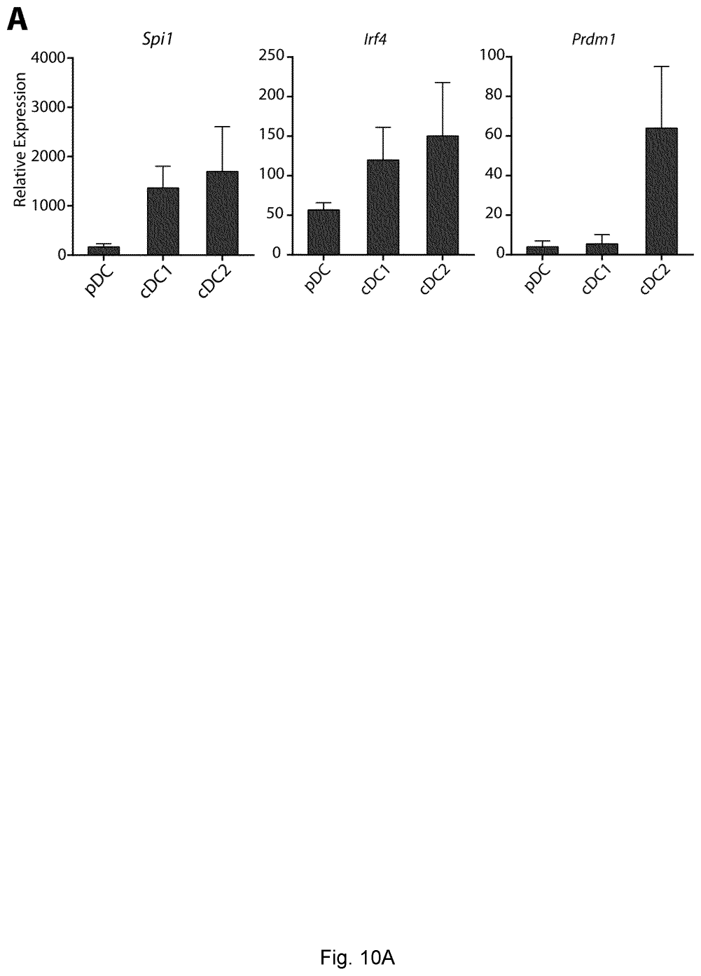

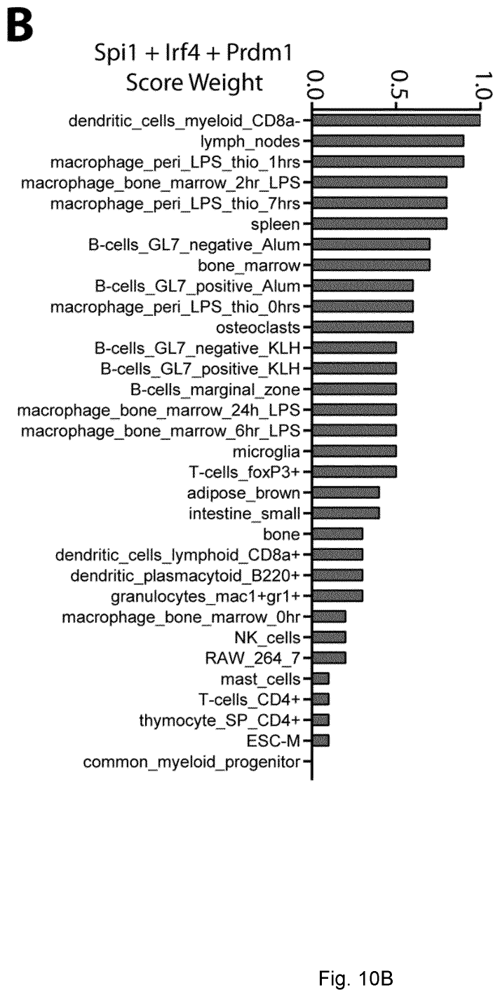

Comparing Spi1, Irf4 and Prdm1 individual expression reveals an enrichment of all these three TFs in cDC2s with PRDM1 displaying the highest expression in cDC2s when compared to other DC populations (FIG. 10A). Combined expression of Spi1, Irf4 and Prdm1 is also highly associated with CD8a⁻ DCs (FIG. 10B).

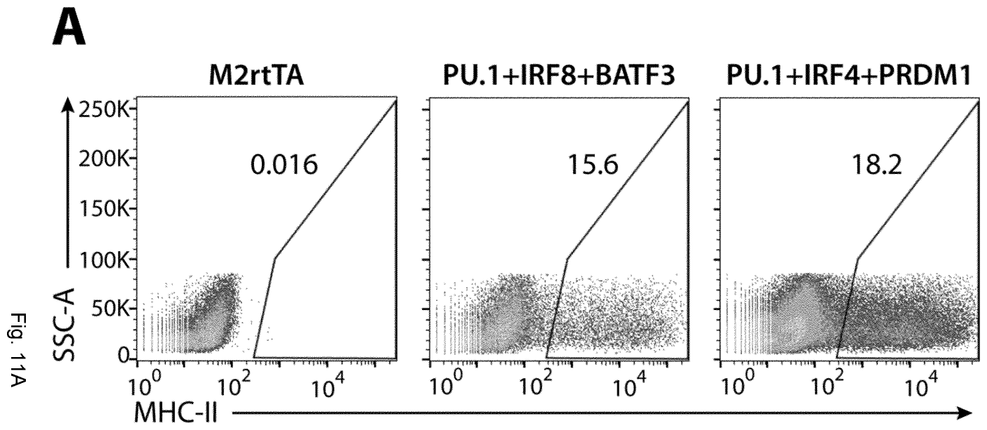

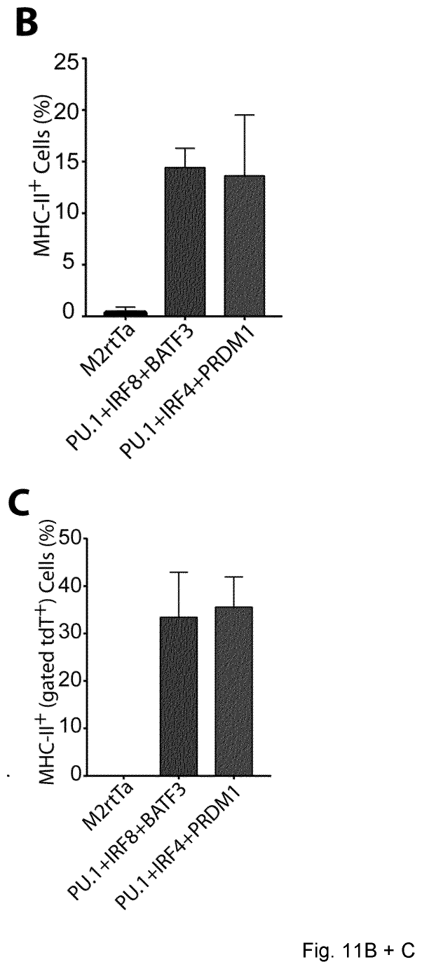

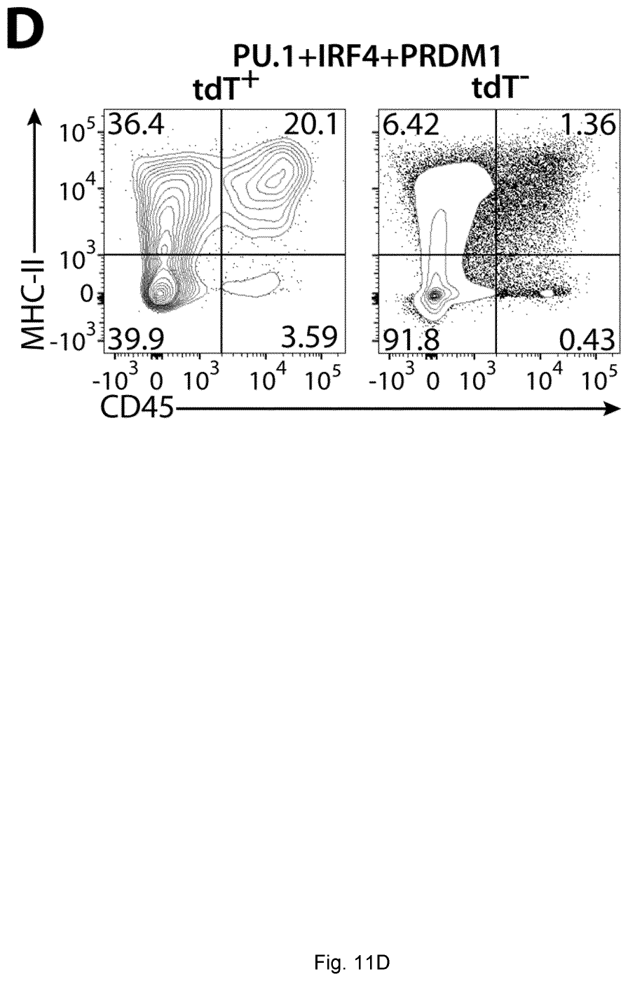

Further analyzing cell surface marker expression, 13.62% of PU.1+IRF4+PRDM1 transduced cells expressed MHC-II, compared with 14.43% of expression in PU.1+IRF8+BATF3 generated DCs (FIGS. 11A and B). Within the tdT⁺ compartment, 35.58% of PU.1+IRF4+PRDM1-induced cells expressed surface MHC-II (FIG. 11C). Additionally, 20.10% of tdT⁺ cells co-expressed surface MHC-II and CD45, while only 1.36% of cells were MHC-II⁺CD45⁺ within the tdT⁻ compartment (FIG. 11D). These data further support that PU.1+IRF4+PRDM1 induces a hematopoietic and APC phenotype and acquisition of antigen presentation machinery.

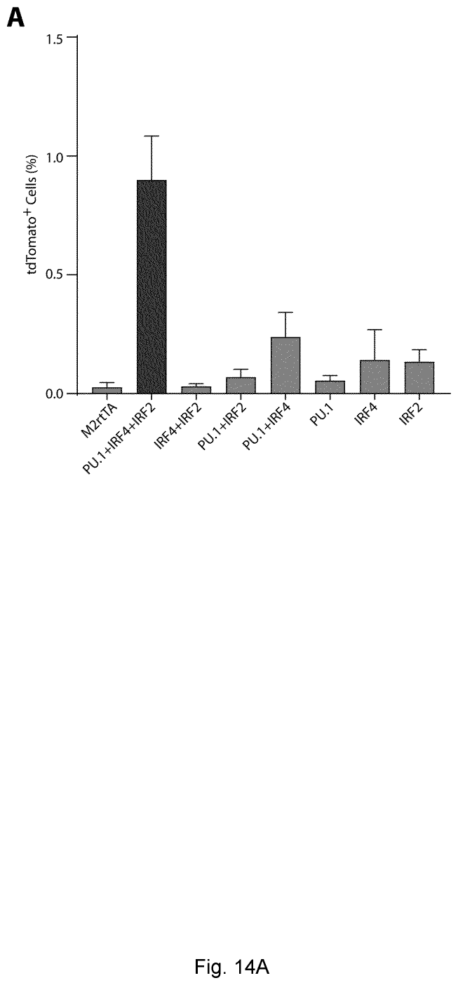

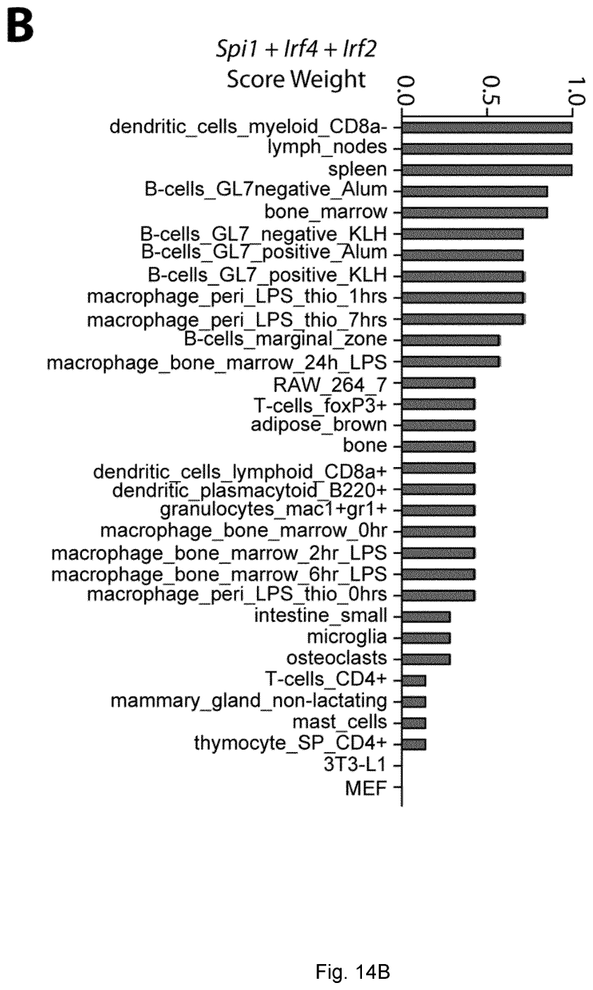

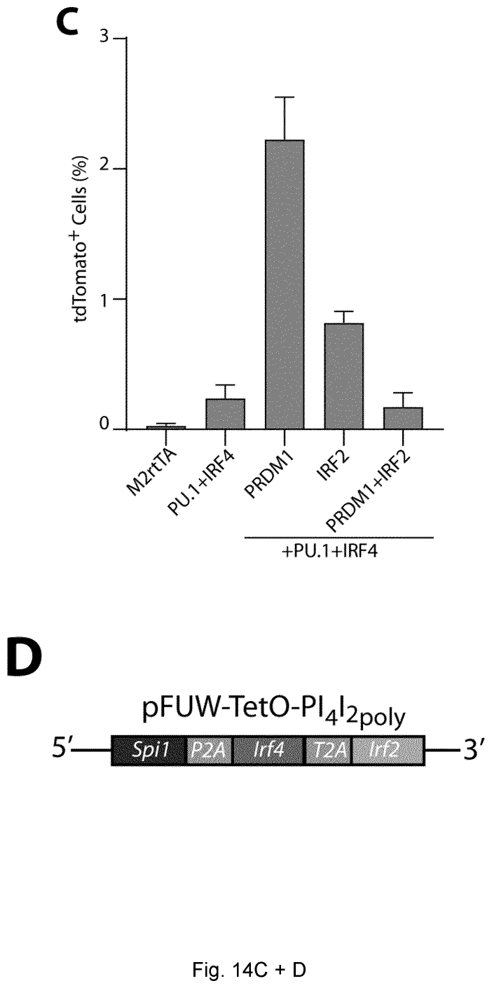

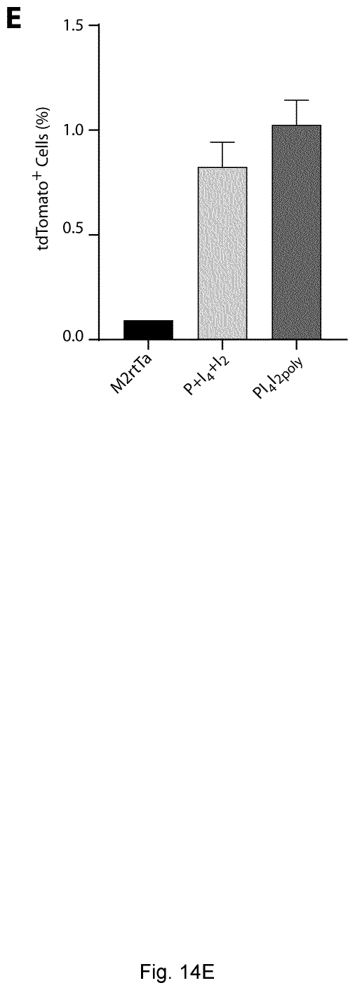

The PU.1+IRF4+IRF2 combination was also further assessed for its role in DC reprogramming. Removal of each of the individual TFs abolished Clec9a reporter activation and individual expression of each TF resulted in low TdT expression (FIG. 14A). Combined expression of Spi1, Irf4 and Irf2 is also highly associated with CD8a-DCs (FIG. 14B). Given that the individual addition of PRDM1 and IRF2 to PU.1 and IRF4 results in a productive Clec9a reporter activation, we investigated the co-expression of these 4 TFs to address a potential synergistic effect. However, overexpression of PU.1, IRF4, IRF2 and PRDM1 abolishes Clec9a reporter activation, suggesting a cross-inhibitory role of PRDM1 and IRF2 in DC reprogramming (FIG. 14C). Collectively, this data implicates PU.1+IRF4+IRF2 as a minimal and sufficient combination to induce a DC phenotype independent of PRDM1, suggesting the induction of a different subset of cDC2.

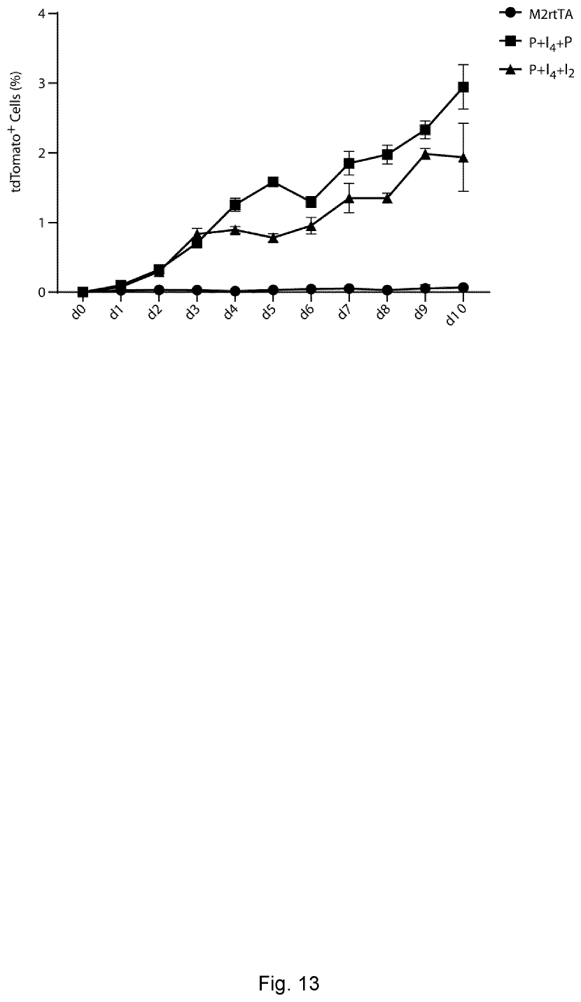

Upon transduction with PU.1, IRF4 and PRDM1 or PU.1, IRF4 and IRF2, activation of the Clec9a reporter starts to be detected at day 2 for both combinations. The peak of TdT expression is reached between day 9 (PU.1+IRF4+IRF2) and day 10 (PU.1, IRF4 and PRDM1) (FIG. 13).

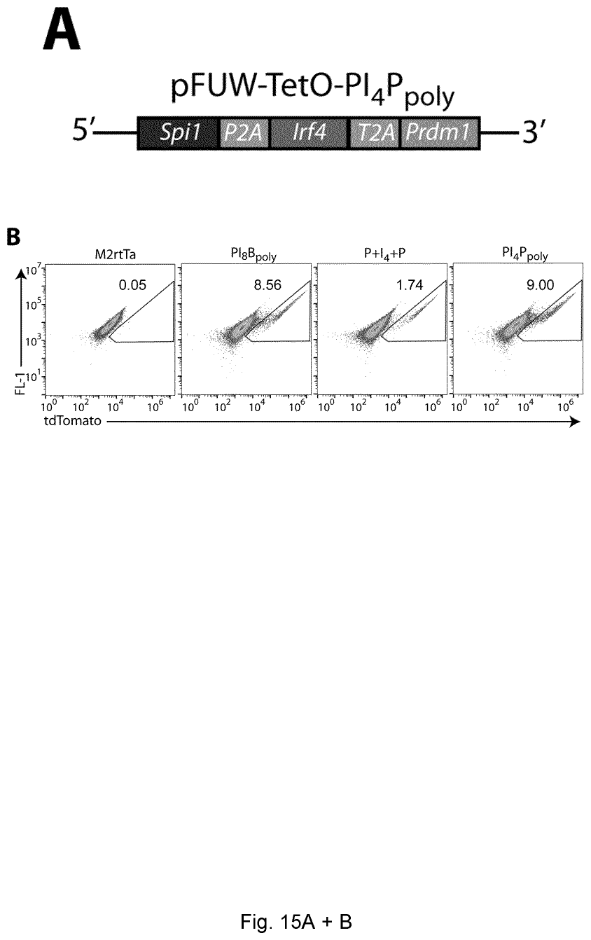

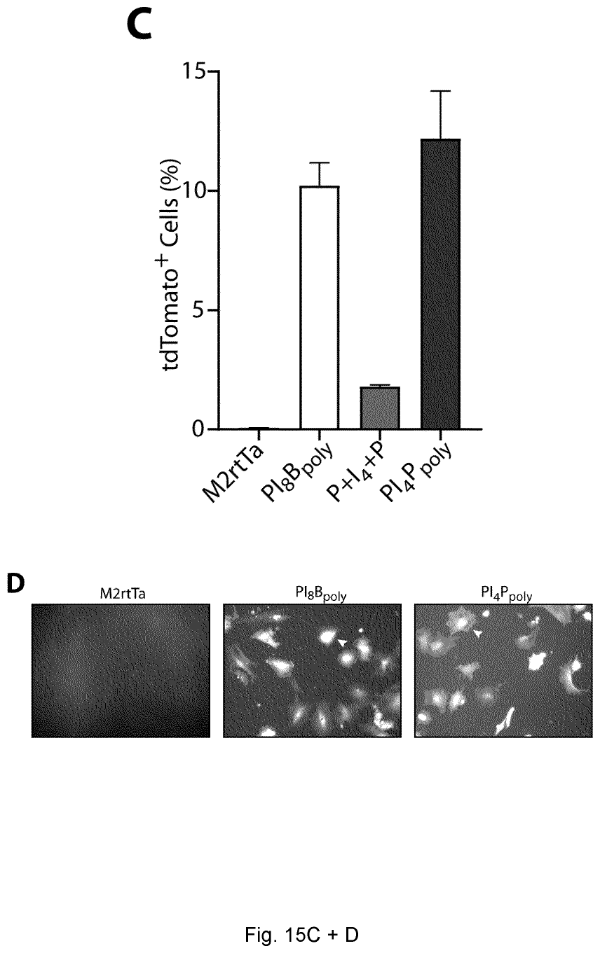

Polycistronic constructs encoding combinations of transcription factors have been used to increase efficiency of reprogramming. Transduction of MEFs with a polycistronic construct encoding Spi1 followed by Irf4 and Irf2 (PI₄I₂Poly) (FIG. 14D) resulted in an increase in the reprogramming efficiency when compared with individual expression (P+I₄+I₂) (FIG. 14E). Additionally, a polycistronic construct was generated encoding Spi1 followed by Irf4 and Prdm1, separated by self-cleaving peptides P2A and T2A (PI₄P_{Poly}) (FIG. 15A). Comparing with individual expression (P+I₄+P), PI₄P_{Poly} resulted in an increase in the reprogramming efficiency reaching 12.20%, a percentage comparable to the previously described polycistronic construct PU.1+IRF8+BATF3 (PI₈B_{Poly}) for cDC1-like reprogramming (FIGS. 15B and 15C). Fluorescence microscopy highlights the dendritic cell morphology of tdT⁺ cells resulting from both PI₈B_{Poly} and PI₄P_{Poly} combinations (FIG. 15C).

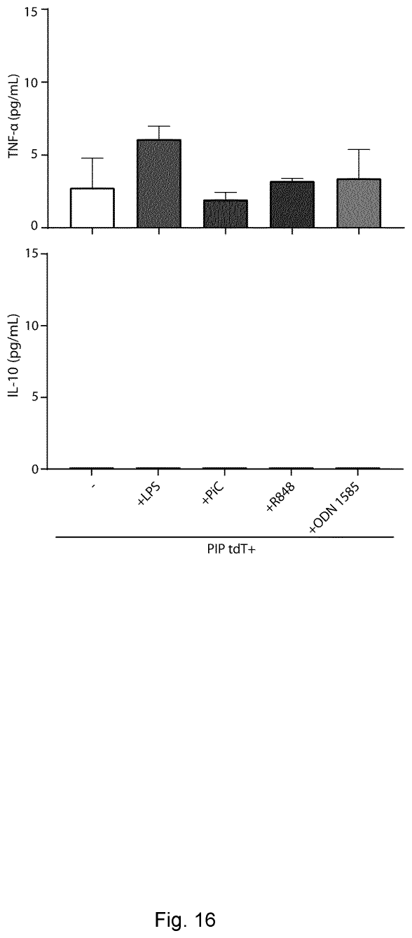

A typical immunomodulatory feature of DCs relays on their ability to secrete cytokines. Pro-inflammatory cDC2 have been described to secrete TNF-α in response to TLR stimuli. Anti-inflammatory cDC2, on the other hand, are characterized by the secretion of IL-10, which further mediates their immunoregulatory functions. Cytokine secretion of sorted PI₈B_{Poly}-generated TdT⁺ cells was performed upon stimulation of toll-like receptors TLR3 (PiC—Polyinosinic:polycytidylic acid), TLR4 (LPS—Lipopolysaccharide), TLR7/TLR8 (R848—Resiquimod) and TLR9 (CpG ODN 1585). Overexpression of PU.1, IRF8 and PRDM1 induces ability to secrete pro-inflammatory tumor necrosis factor-α (TNF-α), which is increased by 2.2-fold after LPS challenge (FIG. 16). Contrastingly, IL-10, an anti-inflammatory cytokine, is not detected. These results suggest that PI₄P-induces cells pro-inflammatory DC2s.

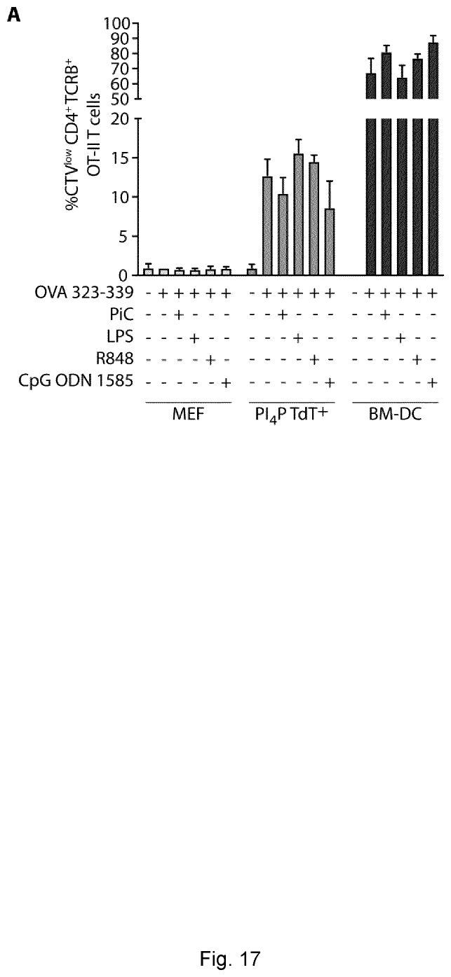

In order to characterize the functional ability of generated cells to promote antigen-specific proliferation of CD4⁺ T cells, day 9 sorted PI₄P_{Poly}-generated TdT⁺ cells, MEFs and bone marrow-derived DCs (BM-DCs) were co-cultured with OT-II CD4⁺ T cells expressing a T cell receptor specific for ovalbumin (OVA) peptide 323-329 presented in the context of MHC-II molecules (FIG. 16). When previously loaded with OVA peptide 323-339, PIP-induced cells acquired the ability to induce proliferation (CTVlow) of 10.67±1.16% OT-II CD4⁺ T cells. In the presence of LPS and R848, PIP-induced cells induce slightly higher proliferation of OT-II CD4⁺ T cells, 12.14±1.33% and 12.23±0.54%, respectively. These data support an ability of PI₄P-induced cells to load and present antigens on MHC-II molecules driving CD4⁺ T cell responses. (FIG. 17)

Recent updates regarding DC heterogeneity have identified two distinct subsets of cDC2 defined by different transcriptional regulators and distinct anti- and pro-inflammatory functions (Brown et al., 2019). Coincidently, in this report, an analysis for the top defining TF genes for each of these newly identified subsets highlights PRDM1 as a top TF associated with cDC2B, a subset characterized by its pro-inflammatory phenotype. Hence, these data further support the pro-inflammatory phenotype of the DCs generated by PU.1+IRF4+PRDM1 reported in the present disclosure, therefore resembling the cDC2B phenotype.

In some embodiments, polypeptide variants or family members having the same or a similar activity as the reference polypeptide encoded by the sequences provided in the sequence list can be used in the compositions, methods, and kits described herein. Generally, variants of a particular polypeptide encoding a cDC2-inducing factor for use in the compositions, methods, and kits described herein will have at least about 95%, at least about 96%, at least about 97%, at least about 98%, at least about 99% or more sequence identity to that particular reference polynucleotide or polypeptide as determined by sequence alignment programs and parameters described herein and known to those skilled in the art.

In an embodiment, Homo sapiens PU.1 transcription factor (PU.1), mRNA (SEQ. ID. 1, SEQ. ID. 2, SEQ. ID. 4, SEQ. ID. 5) and a codon-optimized, or different codons encoding the same amino acids, are naturally also contemplated to be covered by the reference to the nucleic acid as set forth herein.

In an embodiment, Homo sapiens Interferon Regulatory Factor 4 (IRF4), mRNA (SEQ. ID. 7, SEQ. ID. 8, SEQ. ID. 10, SEQ. ID. 11) and a codon-optimized, or different codons encoding the same amino acids, are naturally also contemplated to be covered by the reference to the nucleic acid as set forth herein.

In an embodiment, Homo sapiens PR domain zinc finger protein 1 (PRDM1), mRNA (SEQ. ID. 13, SEQ. ID. 14, SEQ. ID. 16, SEQ. ID. 17) and a codon-optimized, or different codons encoding the same amino acids, are naturally also contemplated to be covered by the reference to the nucleic acid as set forth herein.

In an embodiment, Homo sapiens Interferon Regulatory Factor 2 (IRF2), mRNA (SEQ. ID. 19, SEQ. ID. 20, SEQ. ID. 22, SEQ. ID. 23) and a codon-optimized, or different codons encoding the same amino acids, are naturally also contemplated to be covered by the reference to the nucleic acid as set forth herein.

In an embodiment, Homo sapiens POU class 2 homeobox 2 (POU2F2), mRNA (SEQ. ID. 25, SEQ. ID. 26, SEQ. ID. 28, SEQ. ID. 29) and a codon-optimized, or different codons encoding the same amino acids, are naturally also contemplated to be covered by the reference to the nucleic acid as set forth herein.

In an embodiment, Homo sapiens homeobox protein TGIF1 (TGIF1), mRNA (SEQ. ID. 31, SEQ. ID.32, SEQ. ID. 34, SEQ. ID. 35) and a codon-optimized, or different codons encoding the same amino acids, are naturally also contemplated to be covered by the reference to the nucleic acid as set forth herein.

In an embodiment, Homo sapiens Recombining binding protein suppressor of hairless (RBPJ), mRNA (SEQ. ID. 43, SEQ. ID. 44, SEQ. ID. 46, SEQ. ID. 47) and a codon-optimized, or different codons encoding the same amino acids, are naturally also contemplated to be covered by the reference to the nucleic acid as set forth herein.

In an embodiment, Homo sapiens Transcription factor RelB (RELB), mRNA (SEQ. ID. 37, SEQ. ID. 38, SEQ. ID. 40, SEQ. ID. 41) and a codon-optimized, or different codons encoding the same amino acids, are naturally also contemplated to be covered by the reference to the nucleic acid as set forth herein.

In some embodiments of the compositions, constructs, vectors, methods, and kits provided herein, the number of cDC2-inducing factors used or selected to generate induced cDC2s from a starting somatic cell, such as a fibroblast cell or hematopoietic lineage cell, a multipotent stem cell, an induced pluripotent stem cell, a cancer or tumor cell is at least two. In some embodiments, the number of cDC2-inducing factors used or selected is at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least thirty, at least thirty three, at least thirty five, at least forty, or more.

In some embodiments of the compositions, constructs, vectors, methods, and kits described herein, the nucleic acid sequence or construct encoding the cDC2-inducing factor(s), such as PU.1, IRF4, PRDM1, IRF2, RBPJ, RELB, POU2F2 and TGIF1, is inserted or operably linked into a suitable expression vector for transfection of cells using standard molecular biology techniques. As used herein, a “vector” refers to a nucleic acid molecule, such as a dsDNA molecule that provides a useful biological or biochemical property to an inserted nucleotide sequence, such as the nucleic acid constructs or replacement cassettes described herein. Examples include plasmids, phages, autonomously replicating sequences (ARS), centromeres, and other sequences that are able to replicate or be replicated in vitro or in a host cell, or to convey a desired nucleic acid segment to a desired location within a host cell. A vector can have one or more restriction endonuclease recognition sites (whether type I, II or IIs) at which the sequences can be cut in a determinable fashion without loss of an essential biological function of the vector, and into which a nucleic acid fragment can be spliced or inserted in order to bring about its replication and cloning. Vectors can also comprise one or more recombination sites that permit exchange of nucleic acid sequences between two nucleic acid molecules. Vectors can further provide primer sites, e.g., for PCR, transcriptional and/or translational initiation and/or regulation sites, recombination signals, replicons, additional selectable markers, etc. A vector can further comprise one or more selectable markers suitable for use in the identification of cells transformed with the vector.

In some embodiments of the compositions, methods, constructs, vectors and kits described herein, the expression vector is a viral vector. Some viral-mediated expression methods employ retroviral, adenoviral, lentiviral, herpes viral, pox viral, and adeno-associated viral (AAV) vectors, and such expression methods have been used in gene delivery and are well known in the art.

In some embodiments of the compositions, constructs, vectors, methods, and kits described herein, the viral vector is a retrovirus. Retroviruses provide a convenient platform for gene delivery. A selected gene can be inserted into a vector and packaged in retroviral particles using techniques known in the art. The recombinant virus can then be isolated and delivered to target cells of the subject either in vivo or ex vivo. A number of retroviral systems have been described. See, e.g., U.S. Pat. No. 5,219,740; Miller and Rosman (1989) BioTechniques 7:980-90; Miller, A. D. (1990) Human Gene Therapy 1:5-14; Scarpa et al. (1991) Virology 180:849-52; Burns et al. (1993) Proc. Natl. Acad. Sci. USA 90:8033-37; Boris-Lawrie and Temin (1993) Curr. Opin. Genet. Develop. 3:102-09. In some embodiments of the compositions, methods, and kits described herein, the retrovirus is replication deficient. Retroviral vector systems exploit the fact that a minimal vector containing the 5′ and 3′ LTRs and the packaging signal are sufficient to allow vector packaging, infection and integration into target cells, provided that the viral structural proteins are supplied in trans in the packaging cell line. Fundamental advantages of retroviral vectors for gene transfer include efficient infection and gene expression in most cell types, precise single copy vector integration into target cell chromosomal DNA and ease of manipulation of the retroviral genome.

In some embodiments of the compositions, constructs, vectors, methods, and kits described herein, the viral vector is an adenovirus-based expression vector. Unlike retroviruses, which integrate into the host genome, adenoviruses persist extrachromosomally, thus minimizing the risks associated with insertional mutagenesis (Haj-Ahmad and Graham (1986) J. Virol. 57:267-74; Bett et al. (1993) J. Virol. 67:5911-21; Mittereder et al. (1994) Human Gene Therapy 5:717-29; Seth et al. (1994) J. Virol. 68:933-40; Barr et al. (1994) Gene Therapy 1:51-58; Berkner, K. L. (1988) BioTechniques 6:616-29; and Rich et al. (1993) Human Gene Therapy 4:461-76). Adenoviral vectors infect a wide variety of cells, have a broad host-range, exhibit high efficiencies of infectivity, direct expression of heterologous genes at high levels, and achieve long-term expression of those genes in vivo. The virus is fully infective as a cell-free virion so injection of producer cell lines is not necessary. With regard to safety, adenovirus is not associated with severe human pathology, and the recombinant vectors derived from the virus can be rendered replication defective by deletions in the early-region 1 (“El”) of the viral genome. Adenovirus can also be produced in large quantities with relative ease. Adenoviral vectors for use in the compositions, methods, and kits described herein can be derived from any of the various adenoviral serotypes, including, without limitation, any of the over 40 serotype strains of adenovirus, such as serotypes 2, 5, 12, 40, and 41. The adenoviral vectors used herein are preferably replication-deficient and contain the cDC2-inducing factor of interest operably linked to a suitable promoter.

In some embodiments of the compositions, constructs, vectors, methods, and kits described herein, the nucleic acid sequences encoding the cDC2-inducing factor(s), such as PU.1, IRF4, PRDM1, IRF2, RBPJ, RELB, POU2F2 and TGIF1 are introduced or delivered using one or more inducible lentiviral vectors. Control of expression of cDC2-inducing factors delivered using one or more inducible lentiviral vectors can be achieved, in some embodiments, by contacting a cell having at least one DC-inducing factor in an expression vector under the control of or operably linked to an inducible promoter, with a regulatory agent (e.g., doxycycline) or other inducing agent. When using some types of inducible lentiviral vectors, contacting such a cell with an inducing agent induces expression of the cDC2-inducing factors, while withdrawal of the regulatory agent inhibits expression. When using other types of inducible lentiviral vectors, the presence of the regulatory agent inhibits expression, while removal of the regulatory agent permits expression. As used herein, the term “induction of expression” refers to the expression of a gene, such as a cDC2-inducing factor encoded by an inducible viral vector, in the presence of an inducing agent, for example, or in the presence of one or more agents or factors that cause endogenous expression of the gene in a cell.

In some embodiments of the aspects described herein, a doxycycline (Dox) inducible lentiviral system is used. Unlike retroviruses, lentiviruses are able to transduce quiescent cells making them amenable for transducing a wider variety of hematopoietic cell types. For example, the pFUW-tetO lentivirus system has been shown to transduce primary hematopoietic progenitor cells with high efficiency.

In some embodiments of the methods described herein, the nucleic acid sequences encoding the cDC2-inducing factor(s), such as PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11), PRDM1 (SEQ. ID. 13, SEQ. ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. 25, SEQ. ID. 26, SEQ. ID. 28, SEQ. ID. 29), TGIF1 (SEQ. ID. 31, SEQ. ID.32, SEQ. ID. 34, SEQ. ID. 35), RBPJ (SEQ. ID. 43, SEQ. ID. 44, SEQ. ID. 46, SEQ. ID. 47) and RELB (SEQ. ID. 37, SEQ. ID. 38, SEQ. ID. 40, SEQ. ID. 41) are introduced or delivered using a non-integrating vector (e.g., adenovirus). While integrating vectors, such as retroviral vectors, incorporate into the host cell genome and can potentially disrupt normal gene function, non-integrating vectors control expression of a gene product by extra-chromosomal transcription. Since non-integrating vectors do not become part of the host genome, non-integrating vectors tend to express a nucleic acid transiently in a cell population. This is due in part to the fact that the non-integrating vectors are often rendered replication deficient. Thus, non-integrating vectors have several advantages over retroviral vectors including, but not limited to: (1) no disruption of the host genome, and (2) transient expression, and (3) no remaining viral integration products. Some non-limiting examples of non-integrating vectors for use with the methods described herein include adenovirus, baculovirus, alphavirus, picornavirus, and vaccinia virus. In some embodiments of the methods described herein, the non-integrating viral vector is an adenovirus. Other advantages of non-integrating viral vectors include the ability to produce them in high titers, their stability in vivo, and their efficient infection of host cells.

Nucleic acid constructs and vectors for use in generating induced cDC2s in the compositions, methods, and kits described herein can further comprise, in some embodiments, one or more sequences encoding selection markers for positive and negative selection of cells. Such selection marker sequences can typically provide properties of resistance or sensitivity to antibiotics that are not normally found in the cells in the absence of introduction of the nucleic acid construct. A selectable marker can be used in conjunction with a selection agent, such as an antibiotic, to select in culture for cells expressing the inserted nucleic acid construct. Sequences encoding positive selection markers typically provide antibiotic resistance, i.e., when the positive selection marker sequence is present in the genome of a cell, the cell is sensitive to the antibiotic or agent. Sequences encoding negative selection markers typically provide sensitivity to an antibiotic or agent, i.e., when the negative selection marker is present in the genome of a cell, the cell is sensitive to the antibiotic or agent.

Nucleic acid constructs and vectors for use in making induced cDC2s in the compositions, methods, and kits thereof described herein can further comprise, in some embodiments, other nucleic acid elements for the regulation, expression, stabilization of the construct or of other vector genetic elements, for example, promoters, enhancers, TATA-box, ribosome binding sites, IRES, as known to one of ordinary skill in the art.

In some embodiments of the compositions, constructs, vectors, methods, and kits described herein, the DC-inducing factor(s), such as PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11), PRDM1 (SEQ. ID. 13, SEQ. ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. 25, SEQ. ID. 26, SEQ. ID. 28, SEQ. ID. 29), TGIF1 (SEQ. ID. 31, SEQ. ID.32, SEQ. ID. 34, SEQ. ID. 35) , RBPJ (SEQ. ID. 43, SEQ. ID. 44, SEQ. ID. 46, SEQ. ID. 47) and RELB (SEQ. ID. 37, SEQ. ID. 38, SEQ. ID. 40, SEQ. ID. 41) are provided as synthetic, modified RNAs, or introduced or delivered into a cell as a synthetic, modified RNA, as described in US Patent Publication 2012-0046346-A1, the contents of which are herein incorporated by reference in their entireties. In those embodiments where synthetic, modified RNAs are used to reprogram cells to induced cDC2s according to the methods described herein, the methods can involve repeated contacting of the cells or involve repeated transfections of the synthetic, modified RNAs encoding DC-inducing factors, such as for example, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, or more transfections.

In addition to one or more modified nucleosides, the modified mRNAs for use in the compositions, constructs, vectors, methods, and kits described herein can comprise any additional modifications known to one of skill in the art and as described in US Patent Publications US 2012/0046346 A1 and US 2012/0251618 A1, and PCT Publication WO 2012/019168. Such other components include, for example, a 5′ cap (e.g., the Anti-Reverse Cap Analog (ARCA) cap, which contains a 5′-5′-triphosphate guanine-guanine linkage where one guanine contains an N7 methyl group as well as a 3′-O-methyl group; caps created using recombinant Vaccinia Virus Capping Enzyme and recombinant 2′-O-methyltransferase enzyme, which can create a canonical 5′-5′-triphosphate linkage between the 5′-most nucleotide of an mRNA and a guanine nucleotide where the guanine contains an N7 methylation and the ultimate 5′-nucleotide contains a 2′-O-methyl generating the Cap1 structure); a poly(A) tail (e.g., a poly-A tail greater than 30 nucleotides in length, greater than 35 nucleotides in length, at least 40 nucleotides, at least 45 nucleotides, at least 55 nucleotides, at least 60 nucleotide, at least 70 nucleotides, at least 80 nucleotides, at least 90 nucleotides, at least 100 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides, or more); a Kozak sequence; a 3′ untranslated region (3′ UTR); a 5′ untranslated region (5′ UTR); one or more intronic nucleotide sequences capable of being excised from the nucleic acid, or any combination thereof.

In an embodiment, the modified mRNAs for use in the compositions, constructs, vectors, methods, and kits described herein can further comprise an internal ribosome entry site (IRES). An IRES can act as the sole ribosome binding site, or can serve as one of multiple ribosome binding sites of an mRNA. An mRNA containing more than one functional ribosome binding site can encode several peptides or polypeptides, such as the cDC2-inducing factors described herein, that are translated independently by the ribosomes (“multicistronic mRNA”). When nucleic acids are provided with an IRES, further optionally provided is a second translatable region. Examples of IRES sequences that can be used according to the disclosure include without limitation, those from picornaviruses (e.g. FMDV), pest viruses (CFFV), polioviruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SW) or cricket paralysis viruses (CrPV).

In some embodiments of the compositions, constructs, vectors, methods, and kits described herein, the synthetic, modified RNA molecule comprises at least one modified nucleoside. In some embodiments of the compositions, methods, and kits described herein, the synthetic, modified RNA molecule comprises at least two modified nucleosides.

In some embodiments of the compositions, constructs, vectors, methods, and kits described herein, the modified nucleosides are selected from the group consisting of 5-methylcytosine (5 mC), N6-methyladenosine (m6A), 3,2′-O-dimethyluridine (m4U), 2-thiouridine (s2U), 2′ fluorouridine, pseudouridine, 2′-O-methyluridine methyluridine (Um), 2′deoxy uridine (2′ dU), 4-thiouridine (s4U), 5-methyluridine (m5U), 2′-O-methyladenosine (m6A), N6,2′-O-dimethyladenosine (m6Am), N6,N6,2-O-trimethyladenosine (m62Am), 2′-O-methylcytidine (Cm), 7-methylguanosine (m7G), 2′-O-methylguanosine (Gm), N2,7-dimethylguanosine (m2,7G), N2,N2,7-trimethylguanosine (m2,2,7G), and inosine (I). In some embodiments, the modified nucleosides are 5-methylcytosine (5 mC), pseudouracil, or a combination thereof.

Modified mRNAs need not be uniformly modified along the entire length of the molecule. Different nucleotide modifications and/or backbone structures can exist at various positions in the nucleic acid. One of ordinary skill in the art will appreciate that the nucleotide analogs or other modification(s) can be located at any position(s) of a nucleic acid such that the function of the nucleic acid is not substantially decreased. A modification can also be a 5′ or 3′ terminal modification. The nucleic acids can contain at a minimum one and at maximum 100% modified nucleotides, or any intervening percentage, such as at least 50% modified nucleotides, at least 80% modified nucleotides, or at least 90% modified nucleotides.

In some embodiments, it is preferred, but not absolutely necessary, that each occurrence of a given nucleoside in a molecule is modified (e.g., each cytosine is a modified cytosine e.g., 5-methylcytosine, each uracil is a modified uracil, e.g., pseudouracil, etc.). For example, the modified mRNAs can comprise a modified pyrimidine such as uracil or cytosine. In some embodiments, at least 25%, at least 50%, at least 80%, at least 90% or 100% of the uracil in the nucleic acid are replaced with a modified uracil. It is also contemplated that different occurrences of the same nucleoside can be modified in a different way in a given synthetic, modified RNA molecule. The modified uracil can be replaced by a compound having a single unique structure, or can be replaced by a plurality of compounds having different structures (e.g., 2, 3, 4 or more unique structures). In some embodiments, at least 25%, at least 50%, at least 80%, at least 90% or 100% of the cytosine in the nucleic acid may be replaced with a modified cytosine. The modified cytosine can be replaced by a compound having a single unique structure, or can be replaced by a plurality of compounds having different structures (e.g., 2, 3, 4 or more unique structures) (e.g., some cytosines modified as 5 mC, others modified as 2′-O-methylcytosine or other cytosine analog). Such multi-modified synthetic RNA molecules can be produced by using a ribonucleoside blend or mixture comprising all the desired modified nucleosides, such that when the RNA molecules are being synthesized, only the desired modified nucleosides are incorporated into the resulting RNA molecule encoding the cDC2-inducing factor.

In certain embodiments it is desirable to intracellularly degrade a modified nucleic acid introduced into the cell, for example if precise timing of protein production is desired. Thus, in some embodiments of the compositions, methods, and kits described herein, provided herein are modified nucleic acids comprising a degradation domain, which is capable of being acted on in a directed manner within a cell.

While it is understood that induced cDC2s can be generated by delivery of cDC2-inducing factors in the form of nucleic acid (DNA or RNA) or amino acid sequences, in some embodiments of the compositions, constructs, vectors, methods, and kits described herein, induced cDC2s can be induced using other methods, such as, for example, by treatment of cells with an agent, such as a small molecule or cocktail of small molecules, that induce expression one or more of the cDC2-inducing factors.

Detection of expression of cDC2-inducing factors introduced into cells or induced in a cell population using the compositions, constructs, vectors, methods, and kits described herein, can be achieved by any of several techniques known to those of skill in the art including, for example, Western blot analysis, immunocytochemistry, and fluorescence-mediated detection.

In order to distinguish whether a given combination of DC-inducing factors has generated induced cDC2s, one or more DC activities or parameters can be measured, such as, in some embodiments, differential expression of surface antigens. The generation of induced DCs using the compositions, methods, and kits described herein preferably causes the appearance of the cell surface phenotype characteristic of endogenous cDC2, such as CD45, MHC-II, CD11 b, Sirpa, CD4, ESAM,Clec4a4, Clec10a, Clec12a and Mg12 for example.

DCs are most reliably distinguished from other immune cells by their functional behavior. Functional aspects of cDC2 phenotypes, or cDC2 activities, such as the ability of an induced cDC2s to secrete cytokines can be easily determined by one of skill in the art using routine methods known in the art. In some embodiments of the aspects described herein, functional assays to identify reprogramming factors can be used. For example, in some embodiments, cytokine secretion can be used to confirm immune-modulatory properties of induced cDC2s generated using the compositions, constructs, vectors, methods, and kits described herein.

As used herein, “cellular parameter,” “DC parameter,” or “cytokine secretion” refer to measurable components or qualities of endogenous or natural DCs, particularly components that can be accurately measured. A cellular parameter can be any measurable parameter related to a phenotype, function, or behavior of a cell. Such cellular parameters include, changes in characteristics and markers of a DC or DC population, including but not limited to changes in viability, cell growth, expression of one or more or a combination of markers, such as cell surface determinants, such as receptors, proteins, including conformational or posttranslational modification thereof, lipids, carbohydrates, organic or inorganic molecules, nucleic acids, e.g. mRNA, DNA, global gene expression patterns, etc. Such cellular parameters can be measured using any of a variety of assays known to one of skill in the art. For example, viability and cell growth can be measured by assays such as Trypan blue exclusion, CFSE dilution, and 3H-thymidine incorporation. Expression of protein or polypeptide markers can be measured, for example, using flow cytometric assays, Western blot techniques, or microscopy methods. Gene expression profiles can be assayed, for example, using RNA-sequencing methodologies and quantitative or semi-quantitative real-time PCR assays. A cellular parameter can also refer to a functional parameter or functional activity. While most cellular parameters will provide a quantitative readout, in some instances a semi-quantitative or qualitative result can be acceptable. Readouts can include a single determined value, or can include mean, median value or the variance, etc. Characteristically a range of parameter readout values can be obtained for each parameter from a multiplicity of the same assays. Variability is expected and a range of values for each of the set of test parameters will be obtained using standard statistical methods with a common statistical method used to provide single values.

In some embodiments of the compositions, methods, and kits described herein, additional factors and agents can be used to enhance induced cDC2s reprogramming. For example, factors and agents that modify epigenetic pathways can be used to facilitate reprogramming into induced cDC2s.

Essentially any primary somatic cell type can be used for producing induced cDC2s or reprogramming somatic cells to induced cDC2s according to the presently described compositions, methods, and kits. Such primary somatic cell types also include other stem cell types, including pluripotent stem cells, such as induced pluripotent stem cells (iPS cells); other multipotent stem cells; oligopotent stem cells; and unipotent stem cells. Some non-limiting examples of primary somatic cells useful in the various aspects and embodiments of the methods described herein include, but are not limited to, fibroblast, epithelial, endothelial, neuronal, adipose, cardiac, skeletal muscle, hematopoietic or immune cells, hepatic, splenic, lung, circulating blood cells, gastrointestinal, renal, bone marrow, and pancreatic cells, as well as stem cells from which those cells are derived. The cell can be a primary cell isolated from any somatic tissue including, but not limited to, spleen, bone marrow, blood, brain, liver, lung, gut, stomach, intestine, fat, muscle, uterus, skin, spleen, endocrine organ, bone, etc. The term “somatic cell” further encompasses, in some embodiments, primary cells grown in culture, provided that the somatic cells are not immortalized. Where the cell is maintained under in vitro conditions, conventional tissue culture conditions and methods can be used, and are known to those of skill in the art. Isolation and culture methods for various primary somatic cells are well within the abilities of one skilled in the art.

In some embodiments of these aspects and all such aspects described herein, the somatic cell is a fibroblast cell.

In some embodiments of these aspects and all such aspects described herein, the somatic cell can be a hematopoietic lineage cell.

In some embodiments of these aspects and all such aspects described herein, the somatic cell can be a cancer cell or a tumor cell.

In some embodiments of the compositions, methods, and kits described herein, a somatic cell to be reprogrammed or made into an induced cDC2s cell is a cell of hematopoietic origin. As used herein, the terms “hematopoietic-derived cell,” “hematopoietic-derived differentiated cell,” “hematopoietic lineage cell,” and “cell of hematopoietic origin” refer to cells derived or differentiated from a multipotent hematopoietic stem cell (HSC). Accordingly, hematopoietic lineage cells for use with the compositions, methods, and kits described herein include multipotent, oligopotent, and lineage-restricted hematopoietic progenitor cells, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, and lymphocytes (e.g., T-lymphocytes, which carry T-cell receptors (TCRs), B-lymphocytes or B cells, which express immunoglobulin and produce antibodies, NK cells, NKT cells, and innate lymphocytes). As used herein, the term “hematopoietic progenitor cells” refer to multipotent, oligopotent, and lineage-restricted hematopoietic cells capable of differentiating into two or more cell types of the hematopoietic system, including, but not limited to, granulocytes, monocytes, erythrocytes, megakaryocytes, and lymphocytes B-cells and T-cells. Hematopoietic progenitor cells encompass multi-potent progenitor cells (MPPs), common myeloid progenitor cells (CMPs), common lymphoid progenitor cells (CLPs), granulocyte-monocyte progenitor cells (GMPs), and pre-megakaryocyte-erythrocyte progenitor cell. Lineage-restricted hematopoietic progenitor cells include megakaryocyte-erythrocyte progenitor cells (MEP), ProB cells, PreB cells, PreProB cells, ProT cells, double-negative T cells, pro-NK cells, pre-granulocyte/macrophage cells, granulocyte/macrophage progenitor (GMP) cells, and pro-mast cells (ProMCs).

Cells of hematopoietic origin for use in the compositions, methods, and kits described herein can be obtained from any source known to comprise these cells, such as fetal tissues, umbilical cord blood, bone marrow, peripheral blood, mobilized peripheral blood, spleen, liver, thymus, lymph, etc. Cells obtained from these sources can be expanded ex vivo using any method acceptable to those skilled in the art prior to use in with the compositions, methods, and kits for making induced cDC2s described herein. For example, cells can be sorted, fractionated, treated to remove specific cell types, or otherwise manipulated to obtain a population of cells for use in the methods described herein using any procedure acceptable to those skilled in the art. Mononuclear lymphocytes may be collected, for example, by repeated lymphocytophereses using a continuous flow cell separator as described in U.S. Pat. No. 4,690,915, or isolated using an affinity purification step of CLP method, such as flow-cytometry using a cytometer, magnetic separation, using antibody or protein coated beads, affinity chromatography, or solid-support affinity separation where cells are retained on a substrate according to their expression or lack of expression of a specific protein or type of protein, or batch purification using one or more antibodies against one or more surface antigens specifically expressed by the cell type of interest. Cells of hematopoietic origin can also be obtained from peripheral blood. Prior to harvest of the cells from peripheral blood, the subject can be treated with a cytokine, such as e.g., granulocyte-colony stimulating factor, to promote cell migration from the bone marrow to the blood compartment and/or promote activation and/or proliferation of the population of interest. Any method suitable for identifying surface proteins, for example, can be employed to isolate cells of hematopoietic origin from a heterogeneous population. In some embodiments, a clonal population of cells of hematopoietic origin, such as lymphocytes, is obtained. In some embodiments, the cells of hematopoietic origin are not a clonal population.

Further, in regard to the various aspects and embodiments of the compositions, methods, and kits described herein, a somatic cell can be obtained from any mammalian species, with non-limiting examples including a murine, bovine, simian, porcine, equine, ovine, or human cell. In some embodiments, the somatic cell is a human cell. In some embodiments, the cell is from a non-human organism, such as a non-human mammal.

In general, the methods for making induced cDC2s described herein involve culturing or expanding somatic cells, such as cells of hematopoietic origin, in any culture medium that is available and well-known to one of ordinary skill in the art. Such media include, but are not limited to, Dulbecco's Modified Eagle's Medium® (DMEM), DMEM F12 Medium®, Eagle's Minimum Essential Medium®, F-12K Medium®, Iscove's Modified Dulbecco's Medium®, RPMI-1640 Medium®, and serum-free medium for culture and expansion of DCs. Many media are also available as low-glucose formulations, with or without sodium. The medium used with the methods described herein can, in some embodiments, be supplemented with one or more immunostimulatory cytokine. Commonly used growth factors include, but are not limited to, G-CSF, GM-CSF, TNF-α, IL-4, IL-3, the Flt-3 ligand and the kit ligand. In addition, in preferred embodiments, the immunostimulatory cytokine is selected from the group consisting of the interleukins (e.g., IL-1α, IL-1β, IL-2, IL-3, IL-4, IL-6, IL-8, IL-9, IL-10, IL-12, IL-18, IL-19, IL-20), the interferons (e.g., IFN-α, IFN-β, IFN-γ), tumor necrosis factor (TNF), transforming growth factor-β (TGF-β), granulocyte colony stimulating factor (G-CSF), macrophage colony stimulating factor (M-CSF), granulocyte-macrophage colony stimulating factor (GM-CSF), the Flt-3 ligand and the kit ligand.

Cells in culture can be maintained either in suspension or attached to a solid support, such as extracellular matrix components or plating on feeder cells, for example. Cells being used in the methods described herein can require additional factors that encourage their attachment to a solid support, in some embodiments, such as type I and type II collagen, chondroitin sulfate, fibronectin, “superfibronectin” and fibronectin-like polymers, gelatin, poly-D and poly-L-lysine, thrombospondin and vitronectin. In some embodiments, the cells are suitable for growth in suspension cultures. Suspension-competent host cells are generally monodisperse or grow in loose aggregates without substantial aggregation. Suspension-competent host cells include cells that are suitable for suspension culture without adaptation or manipulation (e.g., cells of hematopoietic origin, such as lymphoid cells) and cells that have been made suspension-competent by modification or adaptation of attachment-dependent cells (e.g., epithelial cells, fibroblasts).

In some embodiments of these aspects and all such aspects described herein, the isolated induced cDC2s further comprise a pharmaceutically acceptable carrier for administration to a subject in need.

Also provided herein, in some aspects, are methods of treating a subject in need of treatment to induce antigen-specific immune responses to eliminate cancer cells or infectious agents or to generate immune tolerance to self-antigens using the cDC2-inducing compositions and methods of preparing induced cDC2s described herein, or using the isolated induced cDC2s and cell clones thereof produced using any of the combinations of DC-inducing factors, DC-inducing compositions, or methods of preparing induced cDC2s described herein. In such methods of treatment, somatic cells, such as fibroblast cells or hematopoietic lineage cells, can first be isolated from the subject, and the isolated cells transduced or transfected, as described herein with a DC-inducing composition comprising expression vectors or synthetic mRNAs, respectively. The isolated induced cDC2s produced using any of the combinations of cDC2-inducing factors, cDC2-inducing compositions, or methods of preparing induced cDC2s described herein, can then be administered to the subject, such as via systemic injection of the induced cDC2s to the subject.

Also provided herein, in some aspects, are methods of treating a subject in need of treatment to induce antigen-specific immune responses to eliminate cancer cells or infectious agents using the cDC2-inducing compositions and any of the combinations of cDC2-inducing factors described herein. In such methods of treatment, cancer cells are transduced, as described herein with a cDC2-inducing composition comprising expression vectors. Cancer cells can be first isolated from the subject, transduced with a cDC2-inducing composition comprising expression vectors and then administered to the subject, such as via systemic injection. Alternatively, cancers cells can be transduced in situ or in vivo with cDC2-inducing composition comprising viral expression vectors.

The reprogrammed induced cDC2s, generated using the compositions, methods, and kits described herein can, in some embodiments of the methods of treatment described herein, be used directly or administered to subjects in need of immunotherapies. Accordingly, various embodiments of the methods described herein involve administration of an effective amount of induced cDC2s or a population of induced cDC2s, generated using any of the compositions, methods, and kits described herein, to an individual or subject in need of a cellular therapy. The cell or population of cells being administered can be an autologous population or be derived from one or more heterologous sources. Further, such induced cDC2s can be administered in a manner that permits them to migrate to lymph node and activate effector T cells.

The reprogrammed induced cDC2s, generated using the compositions, methods, and kits described herein can, in some embodiments of the methods of treatment described herein, be used directly or administered to subjects suffering from autoimmune or hypersensitivity disorders. Accordingly, various embodiments of the methods described herein involve administration of an effective amount of a induced cDC2s or a population of induced cDC2s, generated using any of the compositions, methods, and kits described herein, to an individual or subject in need of a cellular therapy. The cells or population of cells being administered can be an autologous population or be derived from one or more heterologous sources. Further, such induced cDC2s can be loaded with self-antigens and administered in a manner that permits them to migrate the thymus and promote negative selection of autoreactive T cells, migrate to the lymph nodes and limit effector T cells or promote Treg differentiation.

A variety of means for administering cells to subjects are known to those of skill in the art. Such methods can include systemic injection, for example, i.v. injection, or implantation of cells into a target site in a subject. Cells may be inserted into a delivery device which facilitates introduction by injection or implantation into the subject. Such delivery devices can include tubes, e.g., catheters, for injecting cells and fluids into the body of a recipient subject. In one preferred embodiment, the tubes additionally have a needle, e.g., through which the cells can be introduced into the subject at a desired location. The cells can be prepared for delivery in a variety of different forms. For example, the cells can be suspended in a solution or gel or embedded in a support matrix when contained in such a delivery device. Cells can be mixed with a pharmaceutically acceptable carrier or diluent in which the cells remain viable.

Accordingly, the cells produced by the methods described herein can be used to prepare cells to treat or alleviate several cancers and tumors including, but not limited to, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like.

In addition to the above, the methods of the disclosure can be used to prevent or eliminate infection by pathogens known to predispose to certain cancers. Pathogens of particular interest for use in the cancer vaccines provided herein include the hepatitis B virus (hepatocellular carcinoma), hepatitis C virus (heptomas), Epstein Barr virus (EBV) (Burkitt lymphoma, nasopharynx cancer, PTLD in immunosuppressed individuals), HTLVL (adult T cell leukemia), oncogenic human papilloma viruses types 16, 18, 33, 45 (adult cervical cancer), and the bacterium Helicobacter pylori (B cell gastric lymphoma). Other medically relevant microorganisms that may serve as antigens in mammals and more particularly humans are described extensively in the literature, e.g., C. G. A Thomas, Medical Microbiology, Bailliere Tindall, (1983).

In addition to the above, the methods of the disclosure can be used for viral infections. Exemplary viral pathogens include, but are not limited to, infectious virus that infect mammals, and more particularly humans. Examples of infectious virus include, but are not limited to: Retroviridae (e.g., human immunodeficiency viruses, such as HIV-I (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses such as the SARS coronavirus); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Bir-naviridae;

Hepadnaviridae (Hepatitis B virus); Parvovirida (parvoviruses); Papovaviridae (papilloma viruses, polyoma viruses); Adenoviridae (most adenoviruses); Herpesviridae herpes simplex virus (HSV) 1 and 2, varicella zoster virus, cytomegalovirus (CMV), herpes virus; P.oxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astro viruses).

In addition to the above, the methods of the disclosure can be used to target gram-negative and gram-positive bacteria in vertebrate animals. Such gram positive bacteria include, but are not limited to Pasteurella sp., Staphylococci sp., and Streptococcus sp. Gram negative bacteria include, but are not limited to, Escherichia coli, Pseudomonas sp. , and Salmonella sp. Specific examples of infectious bacteria include but are not limited to: Helicobacter pyloris, Borella burgdorferi, Legionella pneumophilia, Mycobacteria sp. (e.g. M. tuberculosis, M. avium, M. intracellular e, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus infuenzae, Bacillus antracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelii.

In addition to the above, the methods of the disclosure can be used to target pathogens that include, but are not limited to, infectious fungi and parasites that infect mammals, and more particularly humans. Examples of infectious fungi include, but are not limited to: Cryptococcus neoformans, Histoplasma capsulatum, Coccidioides immitis, Blastomyces dermatitidis, Chlamydia trachomatis, and Candida albicans.

In addition to the above, the methods of the disclosure can be used to target parasites such as intracellular parasites and obligate intracellular parasites.

Examples of parasites include but are not limited to Plasmodium falciparum, Plasmodium ovale, Plasmodium malariae, Plasmodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis, Leishmania tropica, Trypanosoma gambiense, Trypanosoma rhodesiense, Wuchereria bancrofti, Brugia malayi, Brugia timori, Ascaris lumbricoides, Onchocerca volvulus and Schistosoma mansoni.

Modified induced cDC2s may be used to induce a tolerogenic response including the suppression of a future or existing immune response, to one or more target antigens. Thus, induced cDC2s are useful for treating or preventing an undesirable immune response including, for example, transplant rejection, graft versus host disease, allergies, parasitic diseases, inflammatory diseases and autoimmune diseases. Examples of transplant rejection, which can be treated or prevented in accordance with the present disclosure, include rejections associated with transplantation of bone marrow and of organs such as heart, liver, pancreas, kidney, lung, eye, skin etc. Examples of allergies include seasonal respiratory allergies; allergy to aeroallergens such as hayfever; allergy treatable by reducing serum IgE and eosinophilia; asthma; eczema; animal allergies, food allergies; latex allergies; dermatitis; or allergies treatable by allergic desensitisation. Autoimmune diseases that can be treated or prevented by the present disclosure include, for example, psoriasis, systemic lupus erythematosus, myasthenia gravis, stiff-man syndrome, thyroiditis, Sydenham chorea, rheumatoid arthritis, diabetes and multiple sclerosis. Examples of inflammatory disease include Crohn's disease, chronic inflammatory eye diseases, chronic inflammatory lung diseases and chronic inflammatory liver diseases, autoimmune haemolytic anaemia, idiopathic leucopoenia, ulcerative colitis, dermatomyositis, scleroderma, mixed connective tissue disease, irritable bowel syndrome, systemic lupus erythromatosus (SLE), multiple sclerosis, myasthenia gravis, Guillan-Barre syndrome (antiphospholipid syndrome), primary myxoedema, thyrotoxicosis, pernicious anaemia, autoimmune atrophic gastris, Addison's disease, insulin-dependent diabetes mellitus (IDDM), Goodpasture's syndrome, Behcet's syndrome, Sjogren's syndrome, rheumatoid arthritis, sympathetic ophthalmia, Hashimoto's disease/hypothyroiditis, celiac disease/dermatitis herpetiformis, and demyelinating disease primary biliary cirrhosis, mixed connective tissue disease, chronic active hepatitis, Graves' disease/hyperthyroiditis, scleroderma, chronic idiopathic thrombocytopenic purpura, diabetic neuropathy and septic shock.

Pharmaceutically acceptable carriers and diluents include saline, aqueous buffer solutions, solvents and/or dispersion media. The use of such carriers and diluents is well known in the art. The solution is preferably sterile and fluid. Preferably, prior to the introduction of cells, the solution is stable under the conditions of manufacture and storage and preserved against the contaminating action of microorganisms such as bacteria and fungi through the use of, for example, parabens, chlorobutanol, phenol, ascorbic acid, thimerosal, and the like.

It is preferred that the mode of cell administration is relatively non-invasive, for example by intravenous injection, pulmonary delivery through inhalation, topical, or intranasal administration. However, the route of cell administration will depend on the tissue to be treated and may include implantation. Methods for cell delivery are known to those of skill in the art and can be extrapolated by one skilled in the art of medicine for use with the methods and compositions described herein.

Also provided herein, in some aspects, are kits for making induced cDC2s, the kits comprising any of the DC-inducing compositions comprising one or more expression vector components described herein.

Also provided herein, in some aspects, are kits comprising one or more of the cDC2-inducing factors described herein as components for the methods of making the induced cDC2s described herein.

Accordingly, in some aspects, provided herein, are kits for preparing induced dendritic cells comprising the following components: (a) one or more expression vectors encoding at least one, two, three, four, five, six, or more cDC2-inducing factors selected from: PU.1 (SEQ. ID. 1, SEQ. ID. 2, SEQ. ID. 4, SEQ. ID. 5), IRF4 (SEQ. ID. 7, SEQ. ID. 8, SEQ. ID. 10, SEQ. ID. 11), PRDM1 (SEQ. ID. 13, SEQ. ID. 14, SEQ. ID. 16, SEQ. ID. 17), IRF2 (SEQ. ID. 19, SEQ. ID. 20, SEQ. ID. 22, SEQ. ID. 23), POU2F2 (SEQ. ID. 25, SEQ. ID. 26, SEQ. ID. 28, SEQ. ID. 29), TGIF1 (SEQ. ID. 31, SEQ. ID. 32, SEQ. ID. 34, SEQ. ID. 35), RBPJ (SEQ. ID. 43, SEQ. ID. 44, SEQ. ID. 46, SEQ. ID. 47), RELB (SEQ. ID. 37, SEQ. ID. 38, SEQ. ID. 40, SEQ. ID. 41) and (b) packaging and instructions therefor.

The kits described herein, in some embodiments, can further provide the synthetic mRNAs or the one or more expression vectors encoding DC-inducing factors in an admixture or as separate aliquots.

In some embodiments, the kits can further comprise an agent to enhance efficiency of reprogramming. In some embodiments, the kits can further comprise one or more antibodies or primer reagents to detect a cell-type specific marker to identify cells induced to the cDC2 state.

In some embodiments, the kits can further comprise a buffer. In some such embodiments, the buffer is RNase-free TE buffer at pH 7.0. In some embodiments, the kit further comprises a container with cell culture medium.

All kits described herein can further comprise a buffer, a cell culture medium, a transduction or transfection medium and/or a media supplement. In preferred embodiments, the buffers, cell culture mediums, transfection mediums, and/or media supplements are DNAse and RNase-free. In some embodiments, the synthetic, modified RNAs provided in the kits can be in a non-solution form of specific quantity or mass, e.g., 20 μg, such as a lyophilized powder form, such that the end-user adds a suitable amount of buffer or medium to bring the components to a desired concentration, e.g., 100 ng/μl.

All kits described herein can further comprise devices to facilitate single-administration or repeated or frequent infusions of the cells generated using the kits components described herein, such as a non-implantable delivery device, e.g., needle, syringe, pen device, or an implantable delivery device, e.g., a pump, a semi-permanent stent (e.g., intravenous, intraperitoneal, intracisternal or intracapsular), or a reservoir. In some such embodiments, the delivery device can include a mechanism to dispense a unit dose of a pharmaceutical composition comprising the induced cDC2s. In some embodiments, the device releases the composition continuously, e.g., by diffusion. In some embodiments, the device can include a sensor that monitors a parameter within a subject. For example, the device can include pump, e.g., and, optionally, associated electronics.

In an embodiment, induced cDC2s are made by the hand of man by, e.g., modifying the gene expression of at least one of the factors disclosed herein of a somatic cell, a pluripotent cell, a progenitor cell or a stem cell, or by exposing any one of these cell types to at least one protein or RNA that produces at least one protein as disclosed herein. The cells can further be made by exposing them to small molecules that turn on at least one of the factors disclosed herein. In some aspects at least two, three, four, five, six factors are used to make the induced cDC2s.

In an embodiment, mouse Embryonic Fibroblasts (MEFs) were isolated and purified in the following way: Clec9aCre/Cre animals (Schram) et al., 2013) were crossed with Rosa26-stopflox-tdTomato reporter mice (The Jackson Laboratory) to generate double homozygous Clec9aCre/Cre RosatdTomato/tdTomato (Clec9a-tdTomato) mice. All animals were housed under controlled temperature (23±2° C.), subject to a fixed 12-h light/dark cycle, with free access to food and water.

In an embodiment, primary cultures of MEFs were isolated from E13.5 embryos of Clec9a-tdTomato or C57BLJ6 mice. Head, fetal liver and all internal organs were removed and the remaining tissue was mechanically dissociated. Dissected tissue was enzymatic digested using 0.12% trypsin/0.1 mM Ethylenediaminetetraacetic acid (EDTA) solution (3 mL per embryo), and incubation at 37° C. for 15 min. Additional 3 mL of same solution per embryo were added, followed by another 15 min incubation period. A single cell suspension was obtained and plated in 0.1% gelatin-coated 10-cm tissue culture dishes in growth media. Cells were grown for 2-3 days until confluence, dissociated with Tryple Express and frozen in Fetal Bovine Serum (FBS) 10% dimethyl sulfoxide (DMSO). Before plating for lentiviral transduction, MEFs were sorted to remove residual CD45⁺ and tdTomato⁺ cells that could represent cells with hematopoietic potential. MEFs used for screening and in the following experiments were tdTomato-CD45⁻ with purity above 99% and expanded up to 4 passages.

In an embodiment, HEK293T cells and MEFs were maintained in growth medium [Dulbecco's modified eagle medium (DMEM) supplemented with 10% (v/v) FBS, 2 mM L-Glutamine and antibiotics (10 μg/ml Penicillin and Streptomycin)]. All cells were maintained at 37° C. and 5% (v/v) CO2. All tissue culture reagents were from Thermo Fisher Scientific unless stated otherwise.

In an embodiment, viral transduction and reprogramming experiments were performed in the following way: Clec9a-tdTomato MEFs were seeded at a density of 40,000 cells per well on 0.1% gelatin coated 6-well plates. Cells were incubated overnight with a ratio of 1:1 FUW-TetO-TFs and FUW-M2rtTA lentiviral particles in growth media supplemented with 8 μg/mL polybrene. When testing combinations of TFs, equal MOls of each individual viral particle were applied. Cells were transduced twice in consecutive days and after overnight incubation, media was replaced with fresh growth media. After the second transduction, growth media was supplemented with Doxycycline (1 μg/mL)—day 0. Media was changed every 2-3 days for the duration of the cultures. Emerging tdTomato+cells were analyzed 5-9 days post-transduction.

In an embodiment, flow cytometry analysis was performed in the following way: transduced Clec9a-tdTomato MEFs were dissociated with TrypLE Express, resuspended in 200 μL PBS 5% FBS and kept at 4° C. prior analysis in BD Accuri C6 (BD Biosciences). For the analysis of MHC-II, CD45 and CD11b cell surface marker expression, dissociated cells were incubated with APC-conjugates rat anti-mouse I-A/I-E, anti-mouse CD45 and anti-mouse CD11b antibodies (Biolegend), respectively, diluted in PBS 5% FBS at 4° C. for 30 minutes in the presence of rat serum (1/100, GeneTex) to block unspecific binding. Cells were washed with PBS 5% FBS, resuspended in PBS 5% FBS and analyzed in a BD Accuri C6. Flow cytometry data were analyzed using FlowJo software (FLOWJO, LLC, version 7.6).

In an embodiment, fluorescence activated cell sorting (FACS) was performed in the following way: To purify Clec9a-tdTomato MEFs, cells were incubated at 4° C. for 30 minutes with APC-Cy7-conjugated anti-CD45 antibody (Biolegend) diluted in PBS 5% FBS. Subsequently, MEFs were washed with PBS 5% FBS, resuspended in PBS 5% FBS and tdTomato-CD45-MEFs were purified in BD FACSAria III (BD Biosciences).

In an embodiment, cytokine secretion analysis was performed the following way: tdT⁺ cells generated by PI4P overexpression were FACS sorting at day 9 of reprogramming. On the following day, overnight stimulation was done by adding LPS (100 ng/mL), PiC (1 μg/mL), R848 (1 μg/mL) or CpG ODN 1585 (0.5 μM) (Invivogen) to the media. Culture supernatants were then collected for further analysis according to the manufacturer's instructions by the LEGENDplex™ Mouse Th Cytokine Panel (13-plex) kit. Acquisition was performed in a BD Accuri C6 and data was then analyzed using the LEGENDplex™ v8.0 software (BioLegend).

In and embodiment, bone marrow was isolated from C57BL6 mice and used to generate bone marrow-derived dendritic cells. Briefly, total bone marrow (BM) cells were harvested from long bones (tibias and femurs) by crushing with pestle and mortar. Cells were harvested in phosphate-buffered saline (PBS) supplemented with 2% FBS and filtered through a 70-μm cell strainer (BD Biosciences). Red blood cells were lysed with BD Pharm Lyse (BD Biosciences) for 8 min at room temperature. Lysis was stopped by the addition of volumes of PBS with 2% FBS. Total BM cells were plated in petri dishes (15×10⁶ cells per 10-cm plate) in RPMI complete media supplemented with Flt3I (200 ng/ml) and GM-CSF (5 ng/ml). After 5 days of culture, 5 ml of complete RPMI media was added, and on day 9, 3×10⁶ cells were replated in 10 ml of fresh media with Flt31 and GM-CSF. BM-DCs were used after 15 days of culture.

In an embodiment, antigen presentation assays were performed the following way: CD4+ T cells were obtained by harvesting spleens of OT-II mice followed by MACS purification with the Miltenyi Naive CD4+ T Cell Isolation Kit. Purified CD4+ T cells were labeled with 5 mM CTV (Thermo Fisher) at room temperature for 20 min, washed and counted. FACS-sorted tdT⁺ PIP-generated cells, MEFs or BM-DCs were previously cultured with OVA 323-339 peptide (10 μg/ml) overnight. After extensive washing, 20,000 tdT⁺ PIP-generated cells, MEFs or BM-DCs were co-cultured with 100,000 CTV-labeled CD4+ T cells in 96-well U-bottom culture plates in the presence or absence of TLR stimuli LPS (100 ng/mL), PiC (1 μg/mL), R848 (1 pg/mL) or CpG ODN 1585 (0.5 μM) (Invivogen). After 5 days of culture, T cells were collected, stained and analyzed in BD LSRFortessa™. T cell proliferation was determined by gating in life single TCRβ⁺ CD4⁺ T-cells.

Those skilled in the art will recognize or be able to ascertain using no more than routine experimentation, many equivalents to the specific embodiments of the invention described herein. The scope of the present invention is not intended to be limited to the above description, but rather is as set forth in the appended claims.

Where singular forms of elements or features are used in the specification of the claims, the plural form is also included, and vice versa, if not specifically excluded. For example, the term “a transcription factor” or “the transcription factor” also includes the plural forms “transcription factors” or “the transcription factors,” and vice versa. In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skilled in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

### EXAMPLES

In order to further characterize the induced cells described in this composition and their similarity to bona fide DC subsets, it is possible to perform mRNA-sequencing at the population level. Population RNA-seq is usually performed the following way: Total RNA is extracted with TRIzol reagent, cDNA is generated by specific RNA kits (e.g. the Takara SMARTSeq Ultra low input RNA kit) and further amplified. The resulting cDNA is then analyzed using the appropriate reagents (e.g. Agilent High sensitivity DNA kit). The resulting library preparation is followed by cDNA tagmentation, addition of forward and reverse indexes by PCR and sequenced on appropriate equipment (e.g. Illumina NextSeq 500).

The resulting data can be then analyzed for differential expression analysis, DC2-specific gene enrichment and integrated with existing public available datasets. Alternatively, single-cell RNA sequencing (scRNA-seq) might complement this analysis by providing expression profiles of individual cells, ideal for a better definition of the phenotypes and cell states of the generated cells here described.

### REFERENCES

Brown, C. C., Gudjonson, H., Pritykin, Y., Deep, D., LavaHee, V. P., Mendoza, A., Fromme, R., Mazutis, L., Ariyan, C., Leslie, C., Pe'er, D., & Rudensky, A. Y. (2019). Transcriptional Basis of Mouse and Human Dendritic Cell Heterogeneity. Cell, 179(4), 846-863.e24. https://doi.org/10.1016/j.ce11.2019.09.035

Datta, J., Terhune, J. H., Lowenfeld, L., Cintolo, J. A., Xu, S., Roses, R. E., & Czerniecki, B. J. (2014). Optimizing dendritic cell-based approaches for cancer immunotherapy. The Yale Journal of Biology and Medicine, 87(4), 491-518. Retrieved from http://www.ncbi.nlm.nih.gov/pubmed/25506283

Gabriele, L., & Ozato, K. (2007). The role of the interferon regulatory factor (IRF) family in dendritic cell development and function. Cytokine & Growth Factor Reviews, 18(5-6), 503-510. https://doi.ord/10.1016/i.cytodfr.2007.06.008

Kopf, M., Gros, G. Le, Bachmann, M., Lamers, M. C., Bluethmann, H., & Kohler, G. (1993). Disruption of the murine IL-4 gene blocks Th2 cytokine responses. Nature, 362(6417), 245-248. https://doi.org/10.1038/362245a0

Laoui, D., Keirsse, J., Morias, Y., Van Overmeire, E., Geeraerts, X., Elkrim, Y., . . . Van Ginderachter, J. A. (2016). The tumour microenvironment harbours ontogenically distinct dendritic cell populations with opposing effects on tumour immunity. Nature Communications, 7, 13720. https://doi.org/10.1038/ncomms13720

Merad, M., Sathe, P., Helft, J., Miller, J., & Mortha, A. (2013). The dendritic cell lineage: ontogeny and function of dendritic cells and their subsets in the steady state and the inflamed setting. Annual Review of Immunology, 31, 563-604. https://doi.org/10.1146/annurev-immuno1-020711-074950

Mosmann, T. R., & Coffman, R. L. (1989). TH1 and TH2 Cells: Different Patterns of Lymphokine Secretion Lead to Different Functional Properties. Annual Review of Immunology, 7(1), 145-173. https://doi.org/10.1146/annurev.iy.07.040189.001045

Pereira, C.-F., Chang, B., Gomes, A., Bernitz, J., Papatsenko, D., Niu, X., . . . Moore, K. A. (2016). Hematopoietic Reprogramming In Vitro Informs In Vivo Identification of Hemogenic Precursors to Definitive Hematopoietic Stem Cells. Developmental Cell, 36(5), 525-539. https://doi.org/10.1016/j.devce1.2016.02.011

Pereira, C.-F., Chang, B., Qiu, J., Niu, X., Papatsenko, D., Hendry, C. E., . . . Moore, K. (2013). Induction of a Hemogenic Program in Mouse Fibroblasts. Cell Stem Cell, 13(2), 205-218. https://doi.org/10.1016/j.stem.2013.05.024

Pereira, C.-F., Lemischka, I. R., & Moore, K. (2012). Reprogramming cell fates: insights from combinatorial approaches. Annals of the New York Academy of Sciences, 1266(1), 7-17. https://doi.org/10.1111/j.1749-6632.2012.06508.x

Plantinga, M., Guilliams, M., Vanheerswynghels, M., Deswarte, K., Branco-Madeira, F., Toussaint, W., . . . Lambrecht, B. N. (2013). Conventional and Monocyte-Derived CD11b+Dendritic Cells Initiate and Maintain T Helper 2 Cell-Mediated Immunity to House Dust Mite Allergen. Immunity, 38(2), 322-335. https://doi.org/10.1016/j.immuni.2012.10.016

Rosa, F. F., Pires, C. F., Kurochkin, I., Ferreira, A. G., Gomes, A. M., Palma, L. G., . . . Pereira, C.-F. (2018). Direct reprogramming of fibroblasts into antigen-presenting dendritic cells. Science Immunology, 3(30), eaau4292. https://doi.org/10.1126/sciimmunol.aau4292

Sakaguchi, S. (2004). Naturally Arising CD4+Regulatory T Cells for Immunologic Self-Tolerance and Negative Control of Immune Responses. Annual Review of Immunology, 22(1), 531-562. https://doi.org/10.1146/annurev.immuno1.21.120601.141122

Schraml, B. U., van Blijswijk, J., Zelenay, S., Whitney, P. G., Filby, A., Acton, S. E., . . . Reis e Sousa, C. (2013). Genetic tracing via DNGR-1 expression history defines dendritic cells as a hematopoietic lineage. Cell, 154(4), 843-858. https://doi.org/10.1016/j.ce11.2013.07.014

Subklewe, M., Geiger, C., Lichtenegger, F. S., Javorovic, M., Kvalheim, G., Schendel, D. J., & Bigalke, I. (2014). New generation dendritic cell vaccine for immunotherapy of acute myeloid leukemia. Cancer Immunology, Immunotherapy : CII, 63(10), 1093-1103. https://doi.org/10.1007/s00262-014-1600-5

Takahashi, K., Tanabe, K., Ohnuki, M., Narita, M., Ichisaka, T., Tomoda, K., & Yamanaka, S. (2007). Induction of Pluripotent Stem Cells from Adult Human Fibroblasts by Defined Factors. Cell, 131(5), 861-872. https://doi.org/10.1016/j.ce11.2007.11.019

Takahashi, K., & Yamanaka, S. (2006). Induction of Pluripotent Stem Cells from Mouse Embryonic and Adult Fibroblast Cultures by Defined Factors. Cell, 126(4), 663-676. https://doi.org/10.1016/j.ce11.2006.07.024

Weaver, C. T., Harrington, L. E., Mangan, P. R., Gavrieli, M., & Murphy, K. M. (2006). Th17: An Effector CD4 T Cell Lineage with Regulatory T Cell Ties. Immunity, 24(6), 677-688. https://doi.org/10.1016/j.immuni.2006.06.002

Xie, H., Ye, M., Feng, R., & Graf, T. (2004). Stepwise reprogramming of B cells into macrophages. Cell, 117(5), 663-676. https://doi.org/10.1016/s0092-8674(04)00419-2

Xu, J., Du, Y., & Deng, H. (2015). Direct lineage reprogramming: strategies, mechanisms, and applications. Cell Stem Cell, 16(2), 119-134. https://doi.org/10.1016/j.stem.2015.01.013

Zhu, J., & Paul, W. E. (2008). CD4 T cells: fates, functions, and faults. Blood, 112(5), 1557-1569. https://doi.org/10.1182/blood-2008-05-078154

1-27. (canceled)

28. A construct or a vector encoding a combination of at least three transcription factors, wherein the combination of encoded transcription factors is selected from the group consisting of :
PU.1, IRF4 and PRDM1;
PU.1, IRF4 and POU2F2;
PU.1, IRF4 and TGIF1; and
PU.1, IRF4 and RBPJ.
and wherein the transcription factors are independently at least 95% identical to a sequence of:
(PU.1) SEQ. ID. 3 or SEQ. ID. 6;
(IRF4) SEQ. ID. 9 or SEQ. ID. 12;
(PRDM1) SEQ. ID. 15 or SEQ. ID. 18;
(POU2F2) SEQ. ID. 27 or SEQ. ID. 30;
(TGIF1) SEQ. ID. 33 or SEQ. ID. 36 and
(RBPJ) SEQ. ID. 45 or SEQ. ID. 48.

29. The construct or vector according to claim 28, wherein the combination of encoded transcription factors is in the following sequential order from 5′ to 3′:
PU.1, IRF4 and PRDM1.

30. The construct or vector according to claim 28, wherein the vector is a viral vector.

31. The construct or vector according to claim 28, wherein the vector is a viral vector selected from the group consisting of: retroviral, adenoviral, lentiviral, herpes viral, pox viral, paramyxoviral, rabdoviral, alphaviral, flaviviral and adeno-associated viral vector.

32. The construct or vector according to claim 28, wherein the construct or vector is synthetic mRNA, naked alphavirus RNA replicons or naked flavivirus RNA replicons.

33. A cell comprising the construct or vector according to claim 28.

34. The cell according to claim 33, wherein the cell is an induced dendritic cell.

35. The cell according to claim 33, wherein the cell is selected from the group consisting of: pluripotent stem cell, multipotent stem cell, differentiated cell, tumor cell, cancer cell and mixtures thereof.

36. A method of treating cancer, infectious diseases or autoimmune diseases, the method comprising administering an effective amount of the construct or vector according to claim 28.

37. A method for reprogramming or inducing a cell into a conventional dendritic cell type 2, comprising the following steps:
transducing the cell with the construct or vector according to claim 28, with one or more vectors encoding at least three transcription factors the first and second being PU.1 and IRF4 and the third being selected from the group consisting of PRDM1, POU2F2, TGIF1 and RBPJ; and
culturing the transduced cell in a cell media that supports growth of dendritic cells or antigen-presenting cells.

38. The method according to claim 37, wherein the transduced cells are cultured during at least 2 days.

39. The method according to claim 37, wherein the cell is selected from the group consisting of: a mammalian cell, a non-human cell or a human cell, a mouse cell, a human or a mouse fibroblast, a mammalian umbilical cord blood stem cell, a pluripotent stem cell or a multipotent stem cell, a differentiated cell, and mixtures thereof, wherein the pluripotent stem cell, multipotent stem cell or differentiated cell, is selected from a group consisting of: an endoderm derived cell, a mesoderm derived cell, or an ectoderm derived cell, a multipotent stem cell including mesenchymal stem cell, a hematopoietic stem cell, an intestinal stem cell, a pluripotent stem cell and a cell line.

40. The method according to 37, wherein the transduction step further comprises transducing the cells with at least one vector selected from the group consisting of: a nucleic acid sequence encoding IL-12; a nucleic acid sequence encoding IL-4; a nucleic acid sequence encoding IFN-α; a nucleic acid sequence encoding IFN-β; a nucleic acid sequence encoding IFN-γ; a nucleic acid sequence encoding TNF; a nucleic acid sequence encoding GM-CSF; a nucleic acid sequence encoding siRNAs targeting IL-10 RNA, and mixtures thereof.

41. The method according to claim 37, wherein the transduction step further comprises transducing the cells with at least one vector comprising nucleic acids encoding immunostimulatory cytokines.

42. A method of treating cancer, infectious diseases or autoimmune diseases, the method comprising administering an effective amount of the cell according to claim 33.

43. A vaccine or an injectable formulation, in particular an in-situ injection, for cancer comprising the construct or vector according to claim 28.

44. A vaccine or an injectable formulation, in particular an in-situ injection, for cancer comprising the cell according to claim 33.
